(19) United States
(12) Patent Application Publication
(10) Pub. No.: US 2021/0284639 A1
(43) Pub. Date: Sep. 16, 2021

(54) TYROSINE AMIDE DERIVATIVES AS RHO- KINASE INHIBITORS

(71) Applicant: CHIESI FARMACEUTICI S.p.A., Parma (IT)

(72) Inventors: Alessandro ACCETTA, Parma (IT); Fabio RANCATI, Parma (IT); Anna Maria CAPELLI, Parma (IT); David Edward CLARK, Parma (IT); Patrizia TISSELLI, Parma (IT); Christine EDWARDS, Parma (IT); Amaud Jean Francois Auguste CHEGUILLAUME, Parma (IT); Gurdip BHALAY, Parma (IT)

(73) Assignee: CHIESI FARMACEUTICI S.p.A., Parma (IT)

(21) Appl. No.: 17/260,631

(22) PCT Filed: Jul. 12, 2019
(86) PCT No.: PCT/EP2019/068832

§ 371 Date: Jan. 15, 2021

(30) Foreign Application Priority Data
Jul. 16, 2018 (EP) 18183733.7

(51) Int. Cl. C07D 471/04; A61K 9/00; A61K 31/437; A61K 45/06; C07D 519/00; A61K 31/496; C07D 487/04; A61K 31/519

(52) U.S. Cl. CPC C07D 471/04; A61K 9/0078; A61K 9/0075; A61K 9/008; A61K 31/519; A61K 45/06; C07D 519/00; A61K 31/496; C07D 487/04; A61K 31/437

(57) ABSTRACT

The invention relates to compounds of formula I inhibiting Rho Kinase that are bicyclic dihydropyrimidine-carboxamide derivatives, methods of preparing such compounds, pharmaceutical compositions containing them and therapeutic use thereof. Particularly the compounds of the invention may be useful in the treatment of many disorders associated with ROCK enzymes mechanisms, such as pulmonary diseases including asthma, chronic obstructive pulmonary disease (COPD), idiopathic pulmonary fibrosis (IPF) and pulmonary arterial hypertension (PAH).

### FIELD OF THE INVENTION

The present invention relates to compounds inhibiting Rho Kinase (hereinafter ROCK Inhibitors); particularly the invention relates to compounds that are tyrosine amide derivatives, methods of preparing such compounds, pharmaceutical compositions containing them and therapeutic use thereof.

The compounds of the invention are inhibitors of the activity or function of the ROCK-I and/or ROCK-II isoforms of the Rho-associated coiled-coil forming protein kinase (ROCK).

### BACKGROUND OF THE INVENTION

Rho-associated coiled-coil forming protein kinase (ROCK) belongs to the AGC (PKA/PKG/PKC) family of serine-threonine kinases. Two human isoforms of ROCK have been described, ROCK-I (also referred to as p160 ROCK or ROKβ) and ROCK-II (ROKα) are approximately 160 kDa proteins containing an N-terminal Ser/Thr kinase domain, followed by a coiled-coil structure, a pleckstrin homology domain, and a cysteine-rich region at the C-terminus (Riento, K.; Ridley, A. J. Rocks: multifunctional kinases in cell behaviour. Nat. Rev. Mol. Cell Biol. 2003, 4, 446-456).

Both ROCK-II and ROCK-I are expressed in many human and rodent tissues including the heart, pancreas, lung, liver, skeletal muscle, kidney and brain (above Riento and Ridley, 2003). In patients with pulmonary hypertension, ROCK activity is significantly higher in both lung tissues and circulating neutrophils as compared with controls (Duong-Quy S, Bei Y, Liu Z, Dinh-Xuan A T. Role of Rho-kinase and its inhibitors in pulmonary hypertension. Pharmacol Ther. 2013; 137(3):352-64). A significant correlation was established between neutrophil ROCK activity and the severity and duration of pulmonary hypertension (Duong-Quy et al., 2013).

There is now substantial evidence that ROCK is involved in many of the pathways that contribute to the pathologies associated with several acute and chronic pulmonary diseases, including asthma, COPD, bronchiectasis and ARDS/ALI. Given the biological effect of ROCK, selective inhibitors have the potential to treat a number of pathological mechanisms in respiratory diseases, such as smooth muscle hyper-reactivity, bronchoconstriction, airway inflammation and airway remodeling, neuromodulation and exacerbations due to respiratory tract viral infection (Fernandes L B, Henry P J, Goldie R G.

Rho kinase as a therapeutic target in the treatment of asthma and chronic obstructive pulmonary disease. Ther Adv Respir Dis. 2007 October; 1(1):25-33). Indeed the Rho kinase inhibitor Y-27632 causes bronchodilatation and reduces pulmonary eosinophilia trafficking and airways hyperresponsiveness (Gosens, R.; Schaafsma, D.; Nelemans, S. A.; Halayko, A. J. Rhokinase as a drug target for the treatment of airway hyperresponsiveness in asthma. Mini-Rev. Med. Chem. 2006, 6, 339-348). Pulmonary ROCK activation has been demonstrated in humans with idiopathic pulmonary fibrosis (IPF) and in animal models of this disease. ROCK inhibitors can prevent fibrosis in these models, and more importantly, induce the regression of already established fibrosis, thus indicating ROCK inhibitors as potential powerful pharmacological agents to halt progression of pulmonary fibrosis (Jiang, C.; Huang, H.; Liu, J.; Wang, Y.; Lu, Z.; Xu, Z. Fasudil, a rho-kinase inhibitor, attenuates bleomycin-induced pulmonary fibrosis in mice. Int. J. Mol. Sci. 2012, 13, 8293-8307).

Various compounds have been described in the literature as Rho Kinase Inhibitors. See e.g. WO2004/039796; WO2006/009889; WO2010/032875; WO2009/079008; WO2014/118133 and WO2018/115383 of the same Applicant.

There remains a potential for developing novel and pharmacologically improved ROCK inhibitors in many therapeutic areas such as: cardiovascular and respiratory diseases, erectile dysfunction, fibrotic diseases, insulin resistance, kidney failure, central nervous system disorders, auto-immune diseases and oncology.

In view of the number of pathological responses which are mediated by ROCK enzymes, there is a continuing need for inhibitors of such enzymes which can be useful in the treatment of many disorders. The present invention relates to novel compounds which are inhibitors of ROCK-I and ROCK-IT isoforms of the Rho-associated coiled-coil forming protein kinase (ROCK) that have therapeutically desirable characteristics, particularly promising for some pulmonary diseases including asthma, chronic obstructive pulmonary disease (COPD), idiopathic pulmonary fibrosis (IPF) and pulmonary hypertension (PH) and specifically pulmonary arterial hypertension (PAH). Our co-pending application n. PCT/EP2018/052009 and the present invention address the above mentioned need by providing such kind of compounds. The compound of the invention are active as inhibitors of ROCK-I and ROCK-II isoforms, they are potent and preferably advantageously show other improved properties such as solubility.

### SUMMARY OF THE INVENTION

The present invention is directed to compounds of formula (I)

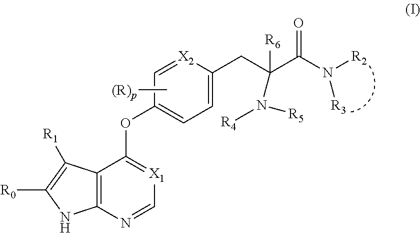

wherein X₁, X₂, R, R₀, R₁, R₂, R₃, R₄, R₅, R₆ and p are as reported below in the detailed description of the invention, acting as ROCK inhibitors, to processes for the preparation thereof, pharmaceutical compositions comprising them either alone or in combination with one or more active ingredient, in admixture with one or more pharmaceutically acceptable carrier.

In one aspect, the present invention refers to a compound of formula (I) for use as a medicament. In one aspect the present invention provides the use of a compound of the invention for the manufacture of a medicament.

In a further aspect, the present invention provides the use of a compound of the invention for the preparation of a medicament for the treatment of any disease characterized by ROCK enzyme aberrant activity and/or wherein an inhibition of activity is desirable and in particular through the selective inhibition of the ROCK enzyme isoforms over other Kinases.

Moreover, the present invention provides a method for prevention and/or treatment of any disease wherein a ROCK enzyme inhibition is desirable, said method comprises administering to a patient in need of such treatment a therapeutically effective amount of a compound of the invention.

In particular the compounds of the invention alone or combined with other active ingredients may be administered for the prevention and/or treatment of a pulmonary disease including asthma, chronic obstructive pulmonary disease (COPD), idiopathic pulmonary fibrosis (IPF) and pulmonary hypertension (PH) and specifically pulmonary arterial hypertension (PAH).

### DETAILED DESCRIPTION OF THE INVENTION

### Definitions

The term “Pharmaceutically acceptable salts” refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts of the invention comprise ions of alkali or alkaline earth metals such as potassium, sodium, calcium or magnesium. Those obtained by reacting the main compound, functioning as a base, with an inorganic or organic acid to form a salt comprise, for example, salts of hydrochloric, hydrobromic, sulfuric, phosphoric, methane sulfonic, camphor sulfonic, acetic, oxalic, maleic, fumaric, succinic and citric acids.

Many organic compounds can form complexes with solvents in which they are reacted or from which they are precipitated or crystallized. These complexes are known as “solvates” which are a further object of the invention. Polymorphs and crystalline forms of compounds of formula (I), or of pharmaceutically acceptable salts, or solvates thereof are a further object of the invention.

The term “Halogen” or “halogen atoms” includes fluorine, chlorine, bromine, and iodine atom, preferably chlorine or fluorine; meaning Fluoro, Chloro, Bromo, Iodo as substituent.

The term “(C₁-C₆) Alkyl” refers to straight-chained or branched alkyl groups wherein the number of constituent carbon atoms is in the range 1 to 6. Particular alkyl groups are methyl, ethyl, n-propyl, isopropyl and t-butyl.

The expressions “(C₁-C₆) Haloalkyl” refer to the above defined “(C₁-C₆)alkyl” groups wherein one or more hydrogen atoms are replaced by one or more halogen atoms, which can be the same or different from each other. Examples include halogenated, poly-halogenated and fully halogenated alkyl groups wherein all of the hydrogen atoms are replaced by halogen atoms, e.g. trifluoromethyl or difluoro methyl groups.

Byway of analogy, the terms “(C₁-C₆) Hydroxyalkyl” or “(C₁-C₆) aminoalkyl” refer to the above defined “(C₁-C₆) alkyl” groups wherein one or more hydrogen atoms are replaced by one or more hydroxy (OH) or amino group respectively, examples being hydroxymethyl and aminomethyl and the like.

The definition of aminoalkyl encompasses alkyl groups (i.e. “(C₁-C₆) alkyl” groups) substituted by one or more amino group (—NR₇R₈). An example of aminoalkyl is a mono-aminoalkyl group such as R₇R₈N—(C₁-C₆) alkyl.

With reference to the substituent R₇ and R₈ as above defined and below, when R₇ and R₈ are taken, together with the nitrogen atom they are linked, to form a 4 to 6 membered heterocyclic radical (likewise R₂ and R₃ above), at least one further ring carbon atom in the said heterocyclic radical is optionally replaced by at least one heteroatom (e.g. N, NH, S or O) and/or may bear -oxo (═O) substituent groups. Said heterocyclic radical may be further optionally substituted on any available points in the ring, namely on a carbon atom, or on any heteroatom available for substitution. Substitution on a carbon atom includes spiro disubstitution as well as substitution on two adjacent carbon atoms, in both cases thus form an additional 5 to 6 membered heterocyclic ring. Examples of said heterocycle radicals are 1-pyrrolidinyl, 1-piperidinyl, 1-piperazinyl, 4-morpholinyl, piperazin-4-yl-2-one, 4-methylpiperazine-1-yl, 7-methyl-4,7-diazaspiro[2.5]octan-4-yl, (3aR,6aS)-5-cyclopropylhexahydropyrrolo[3,4-c]pyrrol-2(1H)-yl), (1S,4S)-5-cyclopropyl-2,5-diazabicyclo[2.2.1]heptan-2-yl, 3,4-dihydro-2,7-naphthyridin-2(1H)-yl, 7,8-dihydro-1,6-naphthyridin-6(5H)-yl and the like.

The term “(C₃-C₁₀) Cycloalkyl” likewise “(C₃-C₆) cycloalkyl” refers to saturated cyclic hydrocarbon groups (including the corresponding spiro disubstituted divalent groups) containing the indicated number of ring carbon atoms. Examples include cyclopropyl, cyclobutyl, cyclopentyl, cyclohexyl and cycloheptyl, and polycyclic ring systems such as adamantan-yl.

The term “(C₂-C₆) Alkenyl” refers to straight or branched carbon chains with one or more double bonds, conjugated or not conjugated, in cis or trans configuration, wherein the number atoms is in the range 2 to 6.

By way of analogy, The term “(C₅-C₇) Cycloalkenyl” refers to cyclic hydrocarbon groups containing from 5 to 7 ring carbon atoms and one or two double bonds.

The term “(C₂-C₆) Alkynyl” refers to straight or branched carbon chains with one or more triple bonds wherein the number atoms is in the range 2 to 6.

The term “(C₂-C₆) Hydroxyalkynyl” refers to the above defined “(C₁-C₆) alkynyl” groups wherein one or more hydrogen atoms are replaced by one or more hydroxy (OH) group.

The term “(C₂-C₆) Aminoalkynyl” refers to the above defined “(C₁-C₆) alkynyl” groups wherein one or more hydrogen atoms are replaced by one or more (—NR₇R₈) groups.

The expression “Aryl” refers to mono, bi- or tri-cyclic carbon ring systems which have 6 to 20, preferably from 6 to 15 ring atoms, wherein at least one ring is aromatic. The expression “heteroaryl” refers to mono-, bi- or tri-cyclic ring systems with 5 to 20, preferably from 5 to 15 ring atoms, in which at least one ring is aromatic and in which at least one ring atom is a heteroatom (e.g. N, S or O).

Examples of aryl or heteroaryl monocyclic ring systems include, for instance, phenyl, thienyl, pyrrolyl, pyrazolyl, imidazolyl, isoxazolyl, oxazolyl, isothiazolyl, thiazolyl, pyridinyl, pyrimidinyl, pyrazinyl, triazinyl, furanyl radicals and the like.

Examples of aryl or heteroaryl bicyclic ring systems include naphthalenyl, biphenylenyl, purinyl, pteridinyl, pyrazolopyrimidinyl, benzotriazolyl, benzoimidazole-yl, quinolinyl, isoquinolinyl, indolyl, isoindolyl, benzothiopheneyl, benzodioxinyl, dihydrobenzodioxinyl, indenyl, dihydro-indenyl, dihydrobenzo[1,4]dioxinyl, benzothiazole-2-yl, dihydrobenzodioxepinyl, benzooxazinyl radicals and the like.

Examples of aryl or heteroaryl tricyclic ring systems include fluorenyl radicals as well as benzocondensed derivatives of the aforementioned heteroaryl bicyclic ring systems.

In an analogous manner, The expressions “arylene” and “heteroarylene” refer to divalent groups, such a phenylene, biphenylene and thienylene. Such groups are also commonly named as “arenediyl” or “heteroarenediyl” groups. For example o-phenylene is also named benzene-1,2-diyl. Thienyl-ene is alternatively named thiophenediyl.

The derived expression “(C₃-C₆) heterocycloalkyl” refers to saturated or partially unsaturated monocyclic (C₃-C₆) cycloalkyl groups in which at least one ring carbon atom is replaced by at least one heteroatom (e.g. N, NH, S or O) or may bear an -oxo (═O) substituent group. Said heterocycloalkyl (i.e. heterocyclic radical or group) might be further optionally substituted on the available points in the ring, namely on a carbon atom, or on an heteroatom available for substitution. Substitution on a carbon atom includes spiro disubstitution as well as substitution on two adjacent carbon atoms, in both cases thus form additional condensed 5 to 6 membered heterocyclic ring. Examples of (C₃-C₆) heterocycloalkyl are represented by: oxetanyl, tetrahydro-furanyl, pyrrolidinyl, imidazolidinyl, thiazolidinyl, piperazinyl, piperidinyl, morpholinyl, thiomorpholinyl, dihydro- or tetrahydro-pyridinyl, tetrahydropyranyl, pyranyl, 2H- or 4H-pyranyl, dihydro- or tetrahydrofuranyl, dihydroisoxazolyl, pyrrolidin-2-one-yl, dihydropyrrolyl radicals and the like.

Examples of said heterocycle radicals are 1-pyrrolidinyl, 1-methyl-2-pyrrolidinyl, 1-piperidinyl, 1-piperazinyl, 4-morpholinyl, piperazin-4-yl-2-one, 4-methylpiperazine-1-yl, 1-methylpiperidin-4-yl, 4-metylpiperazine-1-yl-2-one, 7-methyl-2,7-diazaspiro[3.5]nonan-2-yl, 2-methyl-2,9-diazaspiro[5.5]undecan-9-yl, 9-methyl-3,9-diazaspiro[5.5]undecan-3-yl, and (3aR,6aS)-5-methyl-octahydropyrrolo[3,4-c]pyrrol-2-yl.

The term “Aryl (C₁-C₆) alkyl” refers to an aryl ring linked to a straight-chained or branched alkyl groups wherein the number of constituent carbon atoms is in the range from 1 to 6, e.g. phenylmethyl (i.e. benzyl), phenylethyl or phenylpropyl.

Likewise the term “Heteroaryl (C₁-C₆) alkyl” refers to an heteroaryl ring linked to a straight-chained or branched alkyl groups wherein the number of constituent carbon atoms is in the range from 1 to 6, e.g. furanylmethyl.

The term “alkanoyl”, refers to HC(O)— or to alkylcarbonyl groups (e.g. (C₁-C₆)alkylC(O)— wherein the group “alkyl” has the meaning above defined. Examples include formyl, acetyl, propanoyl, butanoyl.

Likewise “(C₁-C₆)alkyl-sulfonyl” refers to a “(C₁-C₆)alkyl-S(O)₂ group wherein alkyl has the meaning above defined. An example of (C₁-C₆)alkyl-sulfonyl is methylsulfonyl, ethylsulfonyl, propylsulfonyl, isopropylsulfonyl.

“Aryl(C₁-C₆)alkyl-sulfonyl” refers to the above defined (C₁-C₆)alkyl-sulfonyl further substituted by an Aryl. An example of Aryl(C₁-C₆)alkyl-sulfonyl is benzylsulfonyl.

The term “carbamoyl” refers to amino carbonyl derived groups represented by the formula —C(O)NR₇R₈, wherein R₇ and R₈ are as defined above including adjacent, vicinal and spiro di-substituted derivatives. Examples are aminocarbonyl, methylaminocarbonyl, methoxyethylaminocarbonyl, piperazine-1-carbonyl, morpholine-N-carbonyl, morpholine-N-carbonyl and N-(2-(dimethylamino)ethyl)aminocarbonyl, N-(2-(dimethylamino)ethyl)-N-methylaminocarbonyl, N-(3-(dimethylamino)propyl)-N-methylaminocarbonyl, 4-methylpiperazine-1-carbonyl, 4-(dimethylamino)piperidin-1-carbonyl, N-(2-(4-methylpiperazin-1-yl)ethyl)aminocarbonyl, (2-morpholino-ethyl)aminocarbonyl, N-methyl-N-(2-morpholino-ethyl) aminocarbonyl, N-(2-(piperidin-1-yl)ethyl) aminocarbonyl, N-methyl-N-(2-(piperidin-1-yl)ethyl)aminocarbonyl, N-(1-methylpiperidin-4-yl-methyl) aminocarbonyl, N-methyl-N-(1-methylpiperidin-4-yl)aminocarbonyl, N-methyl-N-(1-methylpiperidin-4-yl)aminocarbonyl, 5-methyloctahydropyrrolo[3,4-c]pyrrole-2 carbonyl.

The term “hydroxycarbonyl” refers to a terminal group HOC(O)—.

The term “(C₁-C₁₀) alkoxy” or “(C₁-C₁₀) alkoxyl”, likewise “(C₁-C₆) alkoxy” or “(C₁-C₆) alkoxyl” etc., refers to a straight or branched hydrocarbon of the indicated number of carbons, linked to the rest of the molecule through an oxygen bridge. “(C₁-C₆)Alkylthio” refers to the above hydrocarbon linked through a sulfur bridge.

The derived expression “(C₁-C₆) haloalkoxy” or “(C₁-C₆) haloalkoxyl” refers to the above defined haloalkyl, linked through an oxygen bridge. An example of (C₁-C₆) haloalkoxy is trifluoromethoxy.

By analogy, derived expressions “(C₃-C₆) heterocycloalkyloxyl” and “(C₃-C₆) heterocycloalkyl (C₁-C₆) alkoxyl” refer to heterocycloalkyl groups linked through an oxygen bridge and chained heterocycloalkyl-alkoxyl groups respectively. Examples are respectively (piperidin-4-yl)oxy, 1-methylpiperidin-4-yl)oxy, 2-(piperidin-4-yl)ethoxyl, 2-(1-methylpiperidin-4-yl)ethoxy, and 2-(4-morpholino)ethoxy.

The derived expressions “Aryloxyl” and “Aryl (C₁-C₆) alkoxyl” likewise “heteroAryloxyl” and “Heteroaryl (C₁-C₆) alkoxyl” refer to Aryl or Heteroaryl groups linked through an oxygen bridge and chained Aryl-alkoxyl or HeteroAryl-alkoxyl groups. Examples of such are phenyloxy and benzyloxy and pyridinyloxy respectively.

Likewise derived expression “(C₃-C₆) heterocycloalkyl-(C₁-C₆) alkyl” and “(C₃-C₆) cycloalkyl-(C₁-C₆) alkyl” refer to the above defined heterocycloalkyl and cycloalkyl groups linked to the rest of the molecule via an alkyl group of the indicated number of carbons, for example piperidin-4-yl-methyl, cyclohexylethyl.

The derived expression “(C₁-C₆) alkoxy-(C₁-C₆) alkyl” refers to the above defined alkoxy group linked to the rest of the molecule via an alkyl group of the indicated number of carbons, for example methoxymethyl.

The derived expression “(C₁-C₆) alkoxycarbonyl” refers to the above defined alkoxy group linked to the rest of the molecule via a carbonyl group, for example ethoxycarbonyl.

Further derived expression like “(C₁-C₆) alkoxycarbonyl-amino” refers to the above defined alkoxy group linked to the rest of the molecule via a carbonyl group followed by an amino group (—NR₇—), for example tert-butoxy-carbonyl-amino-.

“(C₁-C₆) alkoxycarbonyl (C₃-C₆) heterocycloalkyl (C₁-C₆) alkyl” refers to alkoxy carbonyl heterocycloalkyl substituents enchained in the said order and linked to the rest of the molecule via an alkyl group of the indicated number of carbons. An example is (tert-butyl piperidine-1-carboxylate)-4 yl-methyl.

The derived expression “(C₁-C₆) aminoalkoxyl” refers to (C₁-C₆) aminoalkyl groups as above defined linked through an oxygen bridge, for example (2-(dimethylamino)ethoxy.

The expression “(C₁-C₆) hydroxyalkoxyl” refers to hydroxyalkyl groups as above defined linked to the rest of the molecule through an oxygen bridge, for example hydroxyethoxy.

The derived expression “(C₁-C₆) aminoalkylcarbamoyl” refers to a “carbamoyl” group, as above defined, substituted with a (C₁-C₆) aminoalkyl group (i.e. —C(O)NR₇R₈ wherein e.g. R₈ is an (C₁-C₆) aminoalkyl). An example is 2-(dimethylamino)ethyl carbamoyl.

The term “Aryl alkanoyl”, refers to an “aryl-carbonyl” (i.e. arylC(O)) or arylalkylcarbonyl group [i.e. aryl(C₁-C₆)alkylC(O)—] wherein aryl and alkyl have the meaning above defined. Examples are represented by benzoyl (i.e. phenylcarbonyl), phenylacetyl, phenylpropanoyl or phenylbutanoyl radicals. Likewise “arylsulfonyl” refers to an arylS(O)₂ group wherein aryl has the meaning above defined. An examples is phenylsulfonyl.

The term “Heteroarylsulfonyl” refers to heteroarylS(O)₂ group wherein heteroaryl has the meaning above defined. An examples is pyridinylsulfonyl.

Enchained substituents derive their definition from the composing fragments, like in the above provided definitions, such as “(C₃-C₆) cycloalkyl-carbonyl”, “(C₃-C₆) heterocycloalkyl-carbonyl”, “heteroaryl-carbonyl”; referring to the above defined fragments linked to the rest of the molecule via a carbonyl group. Examples of such groups comprise cyclopropanecarbonyl, pyrrolidine-3-carbonyl, (pyridin-3-yl)carbonyl.

The expression “Saturated, partially unsaturated or aromatic, five or six membered cycloalkane-diyl, arylene-diyl or heterocycle-diyl” refers to suitable disubstituted cycloalkane or heterocycle or aromatic residue with five or six elements including 1,2-, 1,3- or 1,4-benzene-diyl; 2,3-, 3,4-, 4,5- or 5,6-pyridine-diyl; 3,4-, 4,5- or 5,6-pyridazine-diyl; 4,5- or 5,6-pyrimidine-diyl; 2,3-pyrazinediyl; 2,3-, 3,4- or 4,5-thiophene-diyl/furane-diyl/pyrrole-diyl; 4,5-imidazole-diyl/oxazole-diyl/thiazolediyl; 3,4- or 4,5-pyrazole-diyl/isoxazolediyl/isothiazole-diyl their saturated or partially unsaturated analogues and the like. Other non-vicinal disubstituted residues (diradical) are included too, such as 4,6-pyrimidine-diyl and the like.

The expression “Ring system” refers to mono- or bicyclic or polycyclic ring systems which may be saturated, partially unsaturated or unsaturated, such as aryl, (C₃-C₁₀) cycloalkyl, (C₃-C₆)heterocycloalkyl or heteroaryl.

An oxo moiety is represented by (O) as an alternative to the other common representation, e.g. (═O). Thus, in terms of general formula, the carbonyl group is herein preferably represented as —C(O)— as an alternative to the other common representations such as —CO—, —(CO)— or —C(═O)—. In general the bracketed group is a lateral group, not included into the chain, and brackets are used, when deemed useful, to help disambiguating linear chemical formulas; e.g. the sulfonyl group —SO₂— might be also represented as —S(O)₂— to disambiguate e.g. with respect to the sulfinic group —S(O)O—.

When a numerical index is used like in the statement “p is zero or an integer from 1 to 3” the statement (value) “p is zero” means that the substituent R is absent, that is to say there is no substituent R on the ring.

Whenever basic amino or quaternary ammonium groups are present in the compounds of formula I, physiological acceptable anions, selected among chloride, bromide, iodide, trifluoroacetate, formate, sulfate, phosphate, methanesulfonate, nitrate, maleate, acetate, citrate, fumarate, tartrate, oxalate, succinate, benzoate, p-toluenesulfonate, pamoate and naphthalene disulfonate may be present. Likewise, in the presence of acidic groups such as COOH groups, corresponding physiological cation salts may be present as well, for instance including alkaline or alkaline earth metal ions.

Compounds of formula (I) when contain one or more stereogenic center, may exist as optical stereoisomers.

Where the compounds of the invention have at least one stereogenic center, they may accordingly exist as enantiomers. Where the compounds of the invention possess two or more stereogenic centers, they may additionally exist as diastereoisomers. It is to be understood that all such single enantiomers, diastereoisomers and mixtures thereof in any proportion are encompassed within the scope of the present invention. The absolute configuration (R) or (S) for carbon bearing a stereogenic center is assigned on the basis of Cahn-Ingold-Prelog nomenclature rules based on groups' priorities.

“Single stereoisomer”, “single diastereoisomer” or “single enantiomer”, when reported near the chemical name of a compound indicate that the isomer was isolated as single diastereoisomer or enantiomer (e.g via chromatography) but the absolute configuration at the relevant stereogenic center was not determined/assigned.

Atropisomers result from hindered rotation about single bonds where the steric strain barrier to rotation is high enough to allow for the isolation of the conformers (Bringmann G et al, Angew. Chemie Int. Ed. 44 (34), 5384-5427, 2005. doi:10.1002/anie.200462661).

Oki defined atropisomers as conformers that interconvert with a half-life of more than 1000 seconds at a given temperature (Oki M, Topics in Stereochemistry 14, 1-82, 1983).

Atropisomers differ from other chiral compounds in that in many cases they can be equilibrated thermally whereas in the other forms of chirality isomerization is usually only possible chemically.

Separation of atropisomers is possible by chiral resolution methods such as selective crystallization. In an atropo-enantioselective or atroposelective synthesis one atropisomer is formed at the expense of the other. Atroposelective synthesis may be carried out by use of chiral auxiliaries like a Corey Bakshi Shibata (CBS) catalyst, an asymmetric catalyst derived from proline, or by approaches based on thermodynamic equilibration when an isomerization reaction favors one atropisomer over the other.

Racemic forms of compounds of formula (I) as well as the individual atropisomers (substantially free of its corresponding enantiomer) and stereoisomer-enriched atropisomers mixtures are included in the scope of the present invention.

The invention further concerns the corresponding deuterated derivatives of compounds of formula (I).

All preferred groups or embodiments described above and herebelow for compounds of formula I may be combined among each other and apply as well mutatis mutandis.

The invention is directed to a class of compounds acting as inhibitors of the Rho Kinase (ROCK).

Said class of compounds inhibits the activity or function of the ROCK enzyme and more specifically, they are inhibitors of ROCK-I and ROCK-II isoforms of the Rho-associated coiled-coil forming protein kinase (ROCK). The present invention relates to compounds of formula (I)

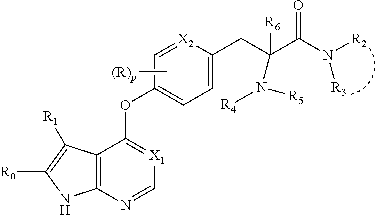

wherein

X₁, and X₂ are in each occurrence independently a CH group or a nitrogen atom.

p is zero or an integer from 1 to 3

each R, when present, is an halogen;

R₀ and R₁ are independently selected from the group consisting of

—H,

(C₁-C₆) alkyl,

(C₃-C₁₀) cycloalkyl,

aryl, heteroaryl and (C₃-C₆) heterocycloalkyl

each of which aryl, heteroaryl and (C₃-C₆) heterocycloalkyl

being in its turn optionally and independently substituted with one or more groups selected from

halogen,

—OH,

R₂ and R₃, the same or different, are selected from the group consisting of

—H,

(C₁-C₆) alkyl,

(C₁-C₆) haloalkyl,

(C₁-C₆) hydroxyalkyl,

(C₁-C₆) aminoalkyl,

(C₁-C₆) alkoxy (C₁-C₆) alkyl,

(C₃-C₁₀)cycloalkyl,

(C₃-C₈)heterocycloalkyl,

aryl,

heteroaryl,

aryl(C₁-C₆)alkyl,

heteroaryl(C₁-C₆)alkyl,

(C₃-C₈)cycloalkyl(C₁-C₆)alkyl,

(C₃-C₈)heterocycloalkyl-(C₁-C₆)alkyl,

each of said aryl, heteroaryl, cycloalkyl, heterocycloalkyl is further optionally substituted by one or more group selected independently from halogen, —CN, —OH, (C₁-C₅)alkyl, (C₃-C₆) cycloalkyl, (C₁-C₆) haloalkyl, (C₁-C₁₀)alkoxy, heterocycloalkyl, aryl, aryl(C₁-C₆)alkyl, —C(O)NR₇R₈, (C₁-C₆) aminoalkyl, (C₁-C₆) hydroxyalkyl, (C₁-C₆) alkoxy (C₁-C₆) alkyl, (C₃-C₈)cycloalkyl(C₁-C₆)alkyl; or

R₂ and R₃, as an alternative, taken together with the nitrogen atom they are linked to, form a mono- or bi-cyclic saturated or partially saturated heterocyclic radical, preferably a 4 to 6 membered monocyclic radical, at least one further ring carbon atom in the said heterocyclic radical is optionally replaced by at least one further heteroatom independently selected from N, NH, S or O and/or may bear an -oxo (═O) substituent group, said heterocyclic radical further optionally including spiro disubstitution as well as substitution on two adjacent or vicinal atoms forming an additional 5 to 6 membered cyclic or heterocyclic, saturated, partially saturated or aromatic, ring;

said heterocyclic radical being optionally further substituted with one or more groups selected from the group consisting of

halogen,

—OH,

—NR₇R₈,

—CH₂NR₇R₈,

(C₁-C₆) alkyl,

(C₁-C₆)alkyl-sulfonyl,

(C₁-C₆) haloalkyl,

(C₁-C₆) hydroxyalkyl,

(C₂-C₆) alkenyl,

(C₂-C₆) alkynyl,

(C₂-C₆) hydroxyalkynyl,

(C₁-C₆) alkoxy (C₁-C₆) alkyl,

(C₁-C₆) alkanoyl,

—C(O)NR₇R₈,

(C₃-C₆) cycloalkyl-carbonyl,

(C₃-C₆) heterocycloalkyl-carbonyl,

aryl(C₁-C₆)alkyl,

aryl alkanoyl,

arylsulfonyl,

aryl(C₁-C₆)alkyl-sulfonyl,

heteroaryl(C₁-C₆)alkyl,

heteroaryl-carbonyl,

heteroarylsulfonyl

heteroaryloxyl,

(C₃-C₆) cycloalkyl including cycloalkyl-yl,

(C₃-C₈)cycloalkyl(C₁-C₆)alkyl

(C₃-C₆) heterocycloalkyl-(C₁-C₆) alkyl,

aryl and heteroaryl

each of said cycloalkyl, aryl and heteroaryl being further optionally substituted by halogen, —OH, (C₁-C₅)alkyl, (C₁-C₆) haloalkyl, (C₁-C₁₀)alkoxy, (C₁-C₆)alkylthio, (C₁-C₆) aminoalkyl, (C₁-C₆) aminoalkoxyl, —C(O)NR₇R₈, (C₁-C₆)alkyl-sulfonyl;

R₄ and R₅ are in each occurrence independently selected in the group consisting of

H,

(C₁-C₆) alkyl,

R₆ is selected from the group consisting of —H, (C₁-C₆) alkyl, (C₁-C₆) haloalkyl;

R₇ and R₈ are in each occurrence independently selected in the group of

H,

(C₁-C₆) alkyl,

(C₁-C₆) haloalkyl,

(C₁-C₆) hydroxyalkyl,

(C₁-C₆) aminoalkyl,

(C₁-C₆) alkoxyl,

(C₁-C₆) alkoxy-(C₁-C₆) alkyl,

(C₃-C₆) heterocycloalkyl-(C₁-C₆) alkyl,

(C₃-C₆) cycloalkyl, aryl, heteroaryl and (C₃-C₆) heterocycloalkyl;

wherein any of said aryl, heteroaryl and (C₃-C₆) heterocycloalkyl in its turn is optionally and independently substituted with one or more groups selected from

halogen,

—OH,

(C₁-C₆) alkyl;

or pharmaceutically acceptable salts and solvates thereof.

In a preferred embodiment, the invention is directed to compounds of formula (I) as above defined wherein each of X₁ and X₂ is a CH; represented by the formula Ia:

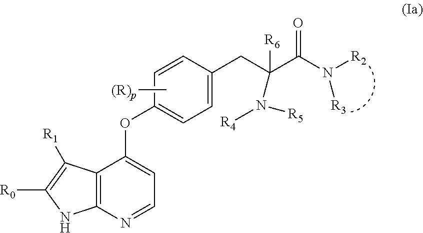

In a second preferred embodiment, the invention is directed to compounds of formula (I) as above defined;

represented by the formula Ic:

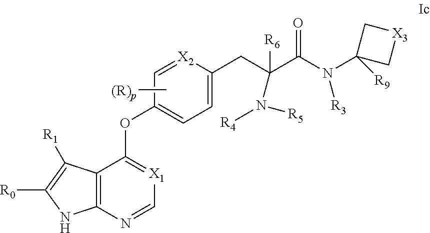

wherein

X₃ is —O— or —(CH₂)ₙ— wherein n is an integer selected from 1, 2 and 3 and

R₉ is selected from the group consisting of

—C(O)NR₇R₈ and (C₁-C₆) hydroxyalkyl;

all the other variables being as defined above.

Preferred in this embodiment are the compounds of formula (Ic) as above defined,

wherein

X₁, is CH or N, and X₂ is a CH group;

p is zero or an integer from 1 to 3

each R, when present, is a halogen;

R₀ is —H, and

R₁ is (C₁-C₆) alkyl,

R₃ is —H,

R₄ and R₅ are both H,

R₆ is —H;

R₉ is-C(O)NR₇R₈, wherein R₇ is H and R₈ is selected from H, (C₁-C₆) alkyl, (C₁-C₆) hydroxyalkyl and (C₁-C₆) alkoxy (C₁-C₆) alkyl;

or pharmaceutically acceptable salt and solvates thereof.

A preferred group of compounds according to the invention are compounds of formula (I)

wherein

X₁ and X₂ are in each occurrence independently a CH group or a nitrogen atom;

p is zero or an integer from 1 to 3;

each R, when present, is fluoro;

R₀ is —H, and R₁ is methyl,

R₃ is —H or methyl and R₂, is independently selected from the group consisting of

—H

methyl,

(C₃-C₁₀)cycloalkyl which is cyclohexyl, cyclobutyl or cyclopentanyl, (C₃-C₈)heterocycloalkyl which is piperidinyl, pyranyl or pyrrolidinyl,

each of said cycloalkyl, heterocycloalkyl is further optionally substituted by one or more group selected independently from (C₁-C₈)alkyl which is methyl, ethyl, isobutyl, tert-butyl, 1-isopropyl; (C₃-C₆) cycloalkyl which is cyclopropyl or cyclobutyl, (C₁-C₆) haloalkyl which is fluoropropyl, heterocycloalkyl which is oxetanyl or tetrahydrofuranyl, —C(O)NR₇R₈ which is aminocarbonyl, methylaminocarbonyl, methoxyethylaminocarbonyl or hydroxyethylaminocarbonyl; (C₁-C₆) hydroxyalkyl which is hydroxyethyl, hydroxymethyl; (C₁-C₆) alkoxy (C₁-C₆) alkyl which is methoxyethyl, (C₃-C₈)cycloalkyl(C₁-C₆)alkyl which is cyclopropylmethyl; or

R₂ and R₃, in the alternative, taken together with the nitrogen atom they are linked to, form a mono-cyclic group which is piperidin-N-yl, pyrrolidin-N-yl, piperazin-N-yl;

or a bi-cyclic group which is 4,7-diazaspiro[2.5]octan-4-yl, (3aR,6aS)-5-cyclopropylhexahydropyrrolo[3,4-c]pyrrol-2(1H)-yl), (1S,4S)-5-cyclopropyl-2,5-diazabicyclo[2.2.1]heptan-2-yl, 3,4-dihydro-2,7-naphthyridin-2(1H)-yl, 5,8-dihydropyrido[3,4-d]pyrimidin-7(6H)-yl, 6,7-dihydrothiazolo[5,4-c]pyridin-5(4H)-yl, 7,8-dihydro-1,6-naphthyridin-6(5H)-yl;

R₄, R₅ and R₆ are —H,

and pharmaceutically acceptable salt and solvates thereof.

The invention also provides a pharmaceutical composition comprising a compound of formula I, or a pharmaceutically acceptable salt thereof in admixture with one or more pharmaceutically acceptable carrier or excipient, either alone or in combination with one or more further active ingredient.

In one aspect the invention provides a compound of formula (I) for use as a medicament.

In a further aspect the invention provides the use of a compound (I), or a pharmaceutically acceptable salt thereof, in the manufacture of a medicament for the treatment of disorders associated with ROCK enzymes mechanisms, particularly for the treatment of disorders such as pulmonary diseases.

In particular the invention provides compounds of formula (I) for use in the prevention and/or treatment of pulmonary disease selected from the group consisting of asthma, chronic obstructive pulmonary disease COPD, idiopathic pulmonary fibrosis (IPF), pulmonary hypertension (PH) and specifically Pulmonary Arterial Hypertension (PAH).

Moreover the invention provides a method for the prevention and/or treatment of disorders associated with ROCK enzymes mechanisms, said method comprising administering to a patient in need of such treatment a therapeutically effective amount of a compound of the invention.

In particular the invention provides methods for the prevention and/or treatment wherein the disorder is asthma, chronic obstructive pulmonary disease COPD idiopathic pulmonary fibrosis (IPF), Pulmonary hypertension (PH) and specifically Pulmonary Arterial Hypertension (PAH).

According to specific embodiments, the invention provides the compounds listed in the table below and pharmaceutical acceptable salts thereof.

| Ex. | |
|---|---|
| No. | Chemical Name |
| 1 | (S)-2-amino-N-(1-cyclobutylpiperidin-4-yl)-3-(3-fluoro-4-((3-methyl-1H- |
| | pyrrolo[2,3-b]pyridin-4-yl)oxy)phenyl)propanamide |
| 2 | (2S)-2-amino-3-(3-fluoro-4-((3-methyl-1H-pyrrolo[2,3-b]pyridin-4- |
| | yl)oxy)phenyl)-N-(1-(tetrahydrofuran-3-yl)piperidin-4-yl)propanamide |
| 3 | (S)-2-amino-3-(3-fluoro-4-((3-methyl-1H-pyrrolo[2,3-b]pyridin-4- |
| | yl)oxy)phenyl)-N-(1-(oxetan-3-yl)piperidin-4-yl)propanamide |
| 4 | (S)-2-amino-N-(1,4-dimethylpiperidin-4-yl)-3-(3-fluoro-4-((3-methyl-1H- |
| | pyrrolo[2,3-b]pyridin-4-yl)oxy)phenyl)propanamide |
| 5 | (S)-2-amino-N-(1-cyclopropylpiperidin-4-yl)-3-(3-fluoro-4-((3-methyl-1H- |
| | pyrrolo[2,3-b]pyridin-4-yl)oxy)phenyl)propanamide |
| 6 | (S)-2-amino-3-(3-fluoro-4-((3-methyl-1H-pyrrolo[2,3-b]pyridin-4- |
| | yl)oxy)phenyl)-N-(1-isobutylpiperidin-4-yl)propanamide |
| 7 | (S)-2-amino-N-(1-ethylpiperidin-4-yl)-3-(3-fluoro-4-43-methyl-1H- |
| | pyrrolo[2,3-b]pyridin-4-yl)oxy)phenyl)propanamide |
| 8 | (2S)-2-amino-3-(3-fluoro-4-((3-methyl-1H-pyrrolo[2,3-b]pyridin-4- |
| | yl)oxy)phenyl)-N-(1,3,3-trimethylpiperidin-4-yl)propanamide |
| 9 | (S)-2-amino-3-(3-fluoro-4-((3-methyl-1H-pyrrolo[2,3-b]pyridin-4- |
| | yl)oxy)phenyl)-1-(4-hydroxy-4-(hydroxymethyl)piperidin-1-yl)propan-1-one |
| 10 | (S)-2-amino-3-(3-fluoro-4-((3-methyl-1H-pyrrolo[2,3-b]pyridin-4- |
| | yl)oxy)phenyl)-N-((R)-1-methylpyrrolidin-3-yl)propanamide |
| 11 | (S)-2-amino-3-(3-fluoro-4-((3-methyl-1H-pyrrolo[2,3-b]pyridin-4- |
| | yl)oxy)phenyl)-1-(7-methyl-4,7-diazaspiro[2.5]otan-4-yl)propan-1-one |
| 12 | (S)-2-amino-1-((3aR,6aS)-5-cyclopropylhexahydropyrrolo[3,4-c]pyrrol-2(1H)-yl)- |
| | 3-(3-fluoro-4((3-methyl-1H-pyrrolo[2,3-b]pyridin-4-yl)oxy)phenyl)propan-1-one |
| 13 | (S)-2-amino-1-((1S,4S)-5-cyclopropyl-2,5-diazabicyclo[2.2.1]heptan-2-yl)-3- |
| | (3-fluoro-4-((3-methyl-1H-pyrrolo[2,3-b]pyridin-4-yl)oxy)phenyl)propan-1-one |
| 14 | (S)-2-amino-3-(3-fluoro-4-((3-methyl-1H-pyrrolo[2,3-b]pyridin-4- |
| | yl)oxy)phenyl)-N-(1-(2-methoxyethyl)piperidin-4-yl)propanamide |
| 15 | (S)-2-amino-3-(3-fluoro-4-((3-methyl-1H-pyrrolo[2,3-b]pyridin-4- |
| | yl)oxy)phenyl)-N-(1-(3-fluoropropyl)piperidin-4-yl)propanamide |
| 16 | (S)-2-amino-N-(1-(cyclopropylmethyl)piperidin-4-yl)-3-(3-fluoro-4-((3-methyl- |
| | 1H-pyrrolo[2,3-b]pyridin-4-yl)oxy)phenyl)propanamide |
| 17 | First eluting diastereoisomer (first diastereoisomer) of (2S)-2-amino-3-(3-fluoro- |
| | 4-((3-methyl-1H-pyrrolo[2,3-b]pyridin-4-yl)oxy)phenyl)-N-(1-methylpiperidin- |
| | 3-yl)propanamide |
| 18 | Second eluting diastereoisomer (second diastereoisomer) of (2S)-2-amino-3-(3- |
| | fluoro-4-((3-methyl-1H-pyrrolo[2,3-b]pyridin-4-yl)oxy)phenyl)-N-(1- |
| | methylpiperidin-3-yl)propanamide |
| 19 | (S)-2-amino-1-(4-(cyclopropylamino)piperidin-1-yl)-3-(3-fluoro-4-((3-methyl- |
| | 1H-pyrrolo[2,3-b]pyridin-4-yl)oxy)phenyl)propan-1-one |
| 20 | (S)-2-amino-N-(1-cyclopropyl-4-(hydroxymethyl)piperidin-4-yl)-3-(3-fluoro-4- |
| | ((3-methyl-1H-pyrrolo[2,3-b]pyridin-4-yl)oxy)phenyl)propanamide |
| 21 | (S)-4-(2-amino-3-(3-fluoro-4-((3-methyl-1H-pyrrolo[2,3-b]pyridin-4- |
| | yl)oxy)phenyl)propanamido)-1-cyclopropylpiperidine-4-carboxamide |
| 22 | (S)-2-amino-1-(3,4-dihydro-2,7-naphthyridin-2(1H)-yl)-3-(3-fluoro-4-((3- |
| | methyl-1H-pyrrolo[2,3-b]pyridin-4-yl)oxy)phenyl)propan-1-one |
| 23 | (S)-2-amino-1-(5,8-dihydropyrido[3,4-d]pyrimidin-7(6H)-yl)-3-(3-fluoro-4-((3- |
| | methyl-1H-pyrrolo[2,3-b]pyridin-4-yl)oxy)phenyl)propan-1-one |
| 24 | (S)-2-amino-1-(6,7-dihydrothiazolo[5,4-c]pyridin-5(4H)-yl)-3-(3-fluoro-4-((3- |
| | methyl-1H-pyrrolo[2,3-b]pyridin-4-yl)oxy)phenyl)propan-1-one |
| 25 | (S)-2-amino-1-(3,4-dihydro-2,6-naphthyridin-2(1H)-yl)-3-(3-fluoro-4-((3- |
| | methyl-1H-pyrrolo[2,3-b]pyridin-4-yl)oxy)phenyl)propan-1-one |
| 26 | (S)-2-amino-1-(7,8-dihydro-1,6-naphthyridin-6(5H)-yl)-3-(3-fluoro-4-((3- |
| | methyl-1H-pyrrolo[2,3-b]pyridin-4-yl)oxy)phenyl)propan-1-one |
| 27 | (S)-1-(2-amino-3-(3-fluoro-4-((3-methyl-1H-pyrrolo[2,3-b]pyridin-4- |
| | yl)oxy)phenyl)propanamido)cyclobutane-1-carboxamide |
| 28 | (S)-1-(2-amino-3-(3-fluoro-4-((3-methyl-1H-pyrrolo[2,3-b]pyridin-4- |
| | yl)oxy)phenyl)propanamido)-N-methylcyclopentane-1-carboxamide |
| 29 | (S)-1-(2-amino-3-(3-fluoro-4-((3-methyl-1H-pyrrolo[2,3-b]pyridin-4- |
| | yl)oxy)phenyl)propanamido)-N-methylcyclohexane-1-carboxamide |
| 30 | (S)-2-amino-3-(3-fluoro-4-((3-methyl-1H-pyrrolo[2,3-b]pyridin-4- |
| | yl)oxy)phenyl)-N-(1-(hydroxymethyl)cyclobutyl)propanamide |
| 31 | (S)-2-amino-3-(3-fluoro-4-((3-methyl-1H-pyrrolo[2,3-b]pyridin-4- |
| | yl)oxy)phenyl)-1-(4-(m-tolylsulfonyl)piperidin-1-yl)propan-1-one |
| 32 | (S)-1-(2-amino-3-(3-fluoro-4-((5-methyl-7H-pyrrolo[2,3-d]pyrimidin-4- |
| | yl)oxy)phenyl)propanamido)cyclohexanecarboxamide |
| 33 | (S)-1-(2-amino-3-(3-fluoro-4-((5-methyl-7H-pyrrolo[2,3-d]pyrimidin-4- |
| | yl)oxy)phenyl)propanamido)-N-methylcyclohexanecarboxamide |
| 34 | (S)-2-amino-3-(3-fluoro-4-((5-methyl-7H-pyrrolo[2,3-d]pyrimidin-4- |
| | yl)oxy)phenyl)-N-(1-(2-hydroxyethyl)cyclohexyl)propanamide |
| 35 | (S)-2-amino-3-(3-fluoro-4-((5-methyl-7H-pyrrolo[2,3-d]pyrimidin-4- |
| | yl)oxy)phenyl)-N-(1-(hydroxymethyl)cyclohexyl)propanamide |
| 36 | (S)-2-amino-3-(3-fluoro-4-((5-methyl-7H-pyrrolo[2,3-d]pyrimidin-4- |
| | yl)oxy)phenyl)-N-(4-(hydroxymethyptetrahydro-2H-pyran-4-yl)propanamide |
| 37 | (S)-2-amino-3-(3-fluoro-4-((5-methyl-7H-pyrrolo[2,3-d]pyrimidin-4- |
| | yl)oxy)phenyl)-1-(4-((4-fluorophenyl)sulfonyl)piperidin-1-yl)propan-1-one |
| 38 | (S)-2-amino-3-(3-fluoro-4-((5-methyl-7H-pyrrolo[2,3-d]pyrimidin-4- |
| | yl)oxy)phenyl)-1-((S)-3-(phenylsulfonyl)pyrrolidin-1-yl)propan-1-one |
| 39 | (S)-2-amino-N-(1-cyclopropylpiperidin-4-yl)-3-(3-fluoro-4-((5-methyl-7H- |
| | pyrrolo[2,3-d]pyrimidin-4-yl)oxy)phenyl)propanamide |
| 40 | (S)-2-amino-3-(4-((3-cyclopropyl-1H-pyrrolo[2,3-b]pyridin-4-yl)oxy)-3- |
| | fluorophenyl)-N-(1-methylpiperidin-4-yl)propanamide |
| 41 | (S)-2-amino-3-(3,5-difluoro-4-((5-methyl-7H-pyrrolo[2,3-d]pyrimidin-4- |
| | yl)oxy)phenyl)-1-(4-(phenylsulfonyl)piperidin-1-yl)propan-1-one |
| 42 | (S)-2-amino-3-(3,5-difluoro-4-((3-methyl-1H-pyrrolo[2,3-b]pyridin-4- |
| | yl)oxy)phenyl)-1-(4-(phenylsulfonyl)piperidin-1-yl)propan-1-one |
| 43 | (S)-1-(2-amino-3-(3-fluoro-4-((3-methyl-1H-pyrrolo[2,3-b]pyridin-4- |
| | yl)oxy)phenyl)propanamido)-N-(2-methoxyethyl)cyclohexanecarboxamide |
| 44 | (S)-1-(2-amino-3-(3-fluoro-4-((3-methyl-1H-pyrrolo[2,3-b]pyridin-4- |
| | yl)oxy)phenyl)propanamido)-N-(2-hydroxyethyl)cyclohexanecarboxamide |
| 45 | (S)-2-amino-3-(3-fluoro-4-((3-methyl-1H-pyrrolo[2,3-b]pyridin-4- |
| | yl)oxy)phenyl)-N,N-dimethylpropanamide |
| 46 | (S)-2-amino-3-(3-fluoro-4-((3-methyl-1H-pyrrolo[2,3-b]pyridin-4- |
| | yl)oxy)phenyl)-N-methylpropanamide |
| 47 | (S)-2-amino-3-(3-fluoro-4-((3-methyl-1H-pyrrolo[2,3-b]pyridin-4- |
| | yl)oxy)phenyl)propanamide |
| 8a | First Eluting single diastereoisomer (first diastereoisomer) of(2S)-2-amino-3-(3- |
| | fluoro-4-((3-methyl-1H-pyrrolo[2,3-b]pyridin-4-yl)oxy)phenyl)-N-(1,3,3- |
| | trimethylpiperidin-4-yl)propanamide |
| 8b | Second Eluting single diastereoisomer (second diastereoisomer) of (2S)-2- |
| | amino-3-(3-fluoro-4-((3-methyl-1 H-pyrrolo[2,3-b]pyeidin-4-yl)oxy)phenyl)-N- |
| | (1,3,3-trimethylpiperidin-4-yl)propanamide |

The compounds of the invention, including all the compounds hereabove listed, can be prepared from readily available starting materials using the following general methods and procedures or by using slightly modified processes readily available to those of ordinary skill in the art. Although a particular embodiment of the present invention may be shown or described herein, those skilled in the art will recognize that all embodiments or aspects of the present invention can be prepared using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimum reaction conditions may vary depending on the particular reactants or solvent used, such conditions can be readily determined by those skilled in the art by routine optimization procedures.

Thus, processes of preparation described below and reported in the following schemes should not be viewed as limiting the scope of the synthetic methods available for the preparation of the compounds of the invention.

In some cases a step is needed in order to mask or protect sensitive or reactive moieties, generally known protective groups (PG) could be employed, in accordance to general principles of chemistry (Protective group in organic syntheses, 3rd ed. T. W. Greene, P. G. M. Wuts).

Processes of preparation described below and reported in the following Schemes should not be viewed as limiting the scope of the synthetic methods available for the preparation of the compounds of the invention.

The compounds of formula I, including all the compounds here above listed, can be usually prepared according to the procedures shown in the schemes below. Where a specific detail or step differs from the general schemes it has been detailed in the specific examples, and/or in additional schemes.

Compounds of formula I contain at least one stereogenic centre, marked with asterisk * in the picture below.

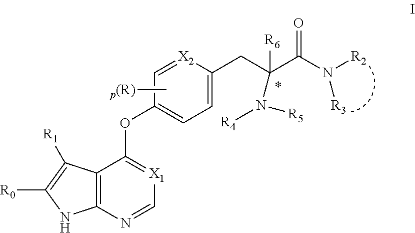

Enantiomerically pure compounds can be prepared according to the reactions described below, by means of enantiomerically pure starting materials and intermediates. Preparation of enantiomerically pure compounds of formula I on the carbon carrying —NR₄R₅ (which is marked with asterisk in the picture above) may be accomplished by means of enantiomerically pure intermediates IV and XII as found in the following schemes. These intermediates may be commercially available or readily produced from commercial sources.

In another approach, enantiomerically pure compounds can be prepared from the corresponding racemates by means of chiral chromatography. Whenever, in compounds of formula I, there are two or more stereogenic centres, the structure is then characterized by different stereoisomers. Stereochemically pure compounds may be obtained by chiral separation from a diastereoisomeric mixture, or stepwise by chromatographic separation of diastereoisomers followed by further chiral separation into single stereoisomers.

Compounds of formula I, wherein R₅ is H, may be prepared according to SCHEME 1 as described hereinafter. SCHEME 1 provides at least one non-limiting synthetic route for the preparation of examples 1 to 39, 41, and 43 to 47.

Typical protective groups (PG₁) for protection of the NH of the 5-membered ring of the bicyclic intermediate II can be 2-[(trimethylsilyl)ethoxy]methyl (SEM), 4-toluenesulfonyl (Ts) and p-methoxybenzyl (PMB), and anyhow not limiting the use of other protective groups. Intermediate III may be prepared from the corresponding intermediate II and a suitable reagent for PG₁ introduction, for example Ts-Cl (tosyl chloride), SEM-Cl ([2-(trimethylsilyl)ethoxy]methyl chloride) or PMB-Br (p-methoxybenzyl bromide). Reaction between said components may be carried out in a polar organic solvent such as DMF, DCM or MeCN, in the presence of a base, such as NaH or DIPEA, at RT or lower.

The carboxylic acid of intermediate IV may be suitably protected as an ester with PG₂ (for example as the methyl ester) and the amino group protected as a carbamate with PG₃ (for example a Boc group). These transformations may be achieved by using generally known methods starting from unprotected tyrosine-like derivatives.

Intermediate V may be obtained from Intermediates III and IV through a palladium catalyzed O-arylation. For example the reaction may be carried out by reacting the aryl halide intermediate III and the phenol derivative IV in a suitable organic solvent such as toluene or THF, in the presence of an inorganic base such as K₂CO₃, with a suitable palladium catalytic system such as Pd₂dba₃/XPhos or another palladium source/phosphine based ligand at high temperature (around 100° C.) for a few hours.

In a different approach, intermediate V may be obtained with a two-step synthesis starting from intermediate VIII. Ipso-substitution of the nitro group of the intermediate VIII by the phenol of intermediate IV, to give intermediate VII, may be carried out in a high boiling organic solvent such as DMSO, at a temperature equal to or higher that 100° C. and in the presence of an inorganic base such as K₂CO₃. Intermediate VII can be converted into intermediate V by removing the chlorine atom by means of heterogeneous palladium catalyzed hydrogenation, by reacting VII under a hydrogen atmosphere, in the presence of Pd/C and an organic base such as TEA. Intermediate VIII may be prepared similarly to intermediate III from a corresponding unprotected heterocycle as described above.

Removal of PG₂ (when PG₂ is a methyl) from intermediate V to give the intermediate VI, whilst not affecting other protections (PG₁: SEM, Ts or PMB and PG₃: Boc), may be carried out by hydrolysis, using an inorganic base such as LiOH or Ba(OH)₂ in a mixture of an organic solvent such as THF and/or methanol with water, usually at RT and for a time ranging from 1 h to overnight. In some cases, for synthetic convenience, the hydrolysis may be carried out at a temperature equal to or higher than 50° C. and may lead to concurrent PG₁ cleavage to give intermediate VIa. Intermediate VIa can be used in a similar way to intermediate VI.

Reaction between intermediate VI (or VIa) and intermediate IX to give intermediate X (or Xa) may be carried out under suitable amide coupling reaction conditions. For example, intermediate VI (or VIa) and IX may be reacted in the presence of an activating agent such as COMU or HATU, with an organic base such as DIPEA or TEA, in a suitable organic solvent such as DCM or DMF, and at temperature usually around RT for a time ranging from a few hours to overnight.

Alternatively, intermediate X may be prepared from intermediate XI and intermediate III through palladium catalyzed O-arylation in a similar way to that described above for the preparation of the intermediate V. In some cases, where X₁═N in Intermediate X, O-arylation may be performed in an alternative condition by heating intermediate III (wherein X₁═N) and intermediate XI in a polar organic solvent such as DMSO in the presence of an inorganic base such as K₂CO₃.

In an alternative approach, intermediate X may be prepared from intermediate XI and intermediate VIII by means of ipso-substitution in a similar way as described above for reaction of intermediate VIII and intermediate IV, followed by hydrogenation as described for intermediate VII to give intermediate V.

Intermediate XI may be obtained by amide coupling of the intermediate XII with intermediate IX in a similar way as described above for the preparation of intermediate X from intermediate VI and IX.

Removal of PG₁ and PG₃ from intermediate X (or Xa, which bears only PG₃), to give compounds of formula I (wherein R₅ is H), may be achieved stepwise or concurrently according to the cleavage conditions used (Protective group in organic syntheses, 3^{rd} ed. T. W. Greene, P. G. M. Wuts). For example, an acidic cleavage using a mixture of TFA in an organic solvent such as DCM, can deprotect both Boc and PMB, while SEM may require an extra treatment in concentrated methanolic ammonia or LiOH. The tosyl group (Ts) may be hydrolysed in a solution of inorganic base such as LiOH in water/methanol at a temperature equal to or higher that 50° C.

R₂ or R₃ substituent of intermediate X may be further elaborated prior to deprotection of PG₁ and PG₃ to give compounds of formula I. For example, if R₂ is a methyl 1-cyclohexanyl carboxylate radical and R₃ is H, the methyl ester of R₂ can be readily converted into a corresponding amide in a two-step process including a methyl ester hydrolysis and an amide coupling.

Thus, the invention is also directed to a process for the preparation of the compounds of general formula I, which process comprises the reaction of a compound of formula VI with a compound of formula IX under amine coupling conditions, followed by removal of the protecting groups.

The invention is also directed to the compound of general formula VI.

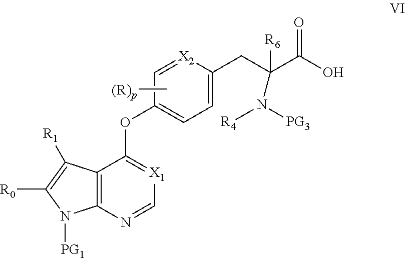

wherein PG₁ and PG₃ are protecting groups.

Preferred are the compounds of formula VI wherein X₁, X₂, R, R₀, R₁, R₄, R₅, R₆ and p are as defined according to the first embodiment of formula (I), or preferably according to the preferred embodiments of formula (Tb) or (Ic).

The invention is also directed the use of compounds of formula VI as intermediates in the preparation of compounds of formula I.

The invention is also directed to the use of VI as intermediate in the preparation of compounds of formula I according to the process as described above.

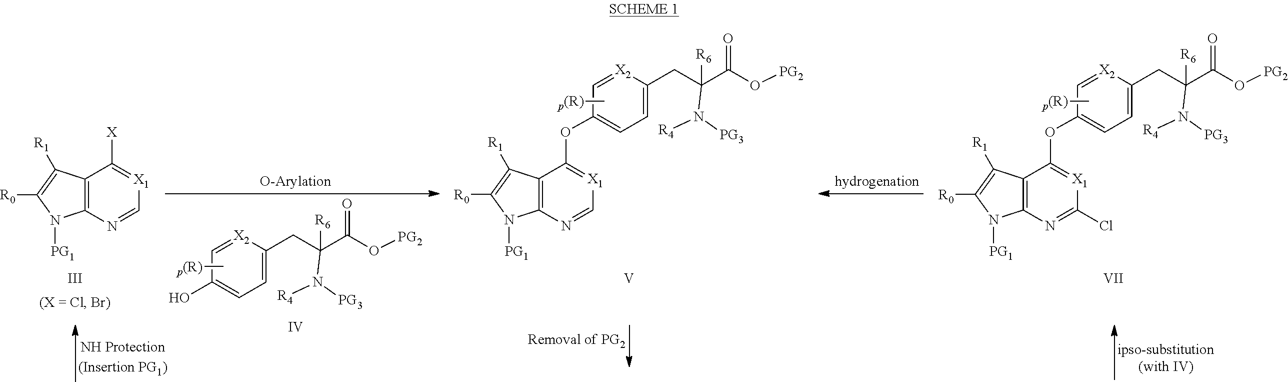

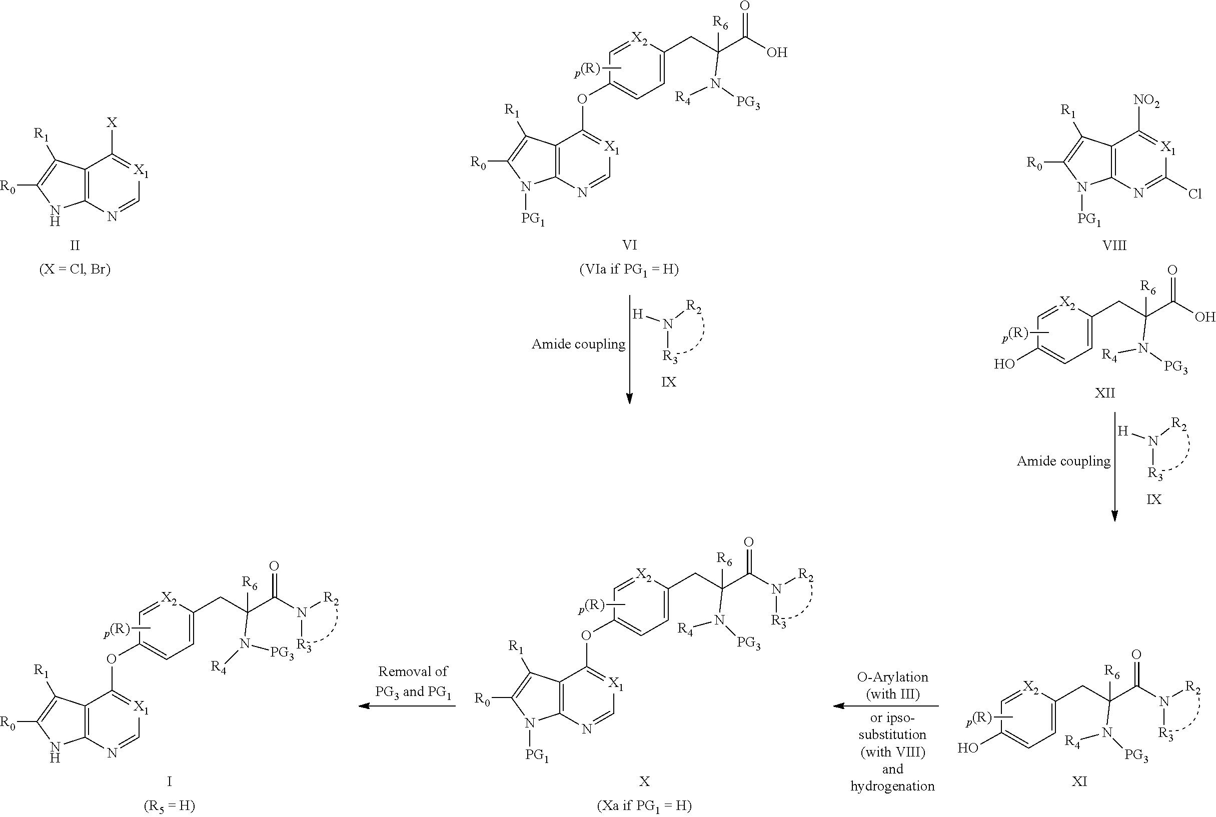

In another approach, compounds of formula I (wherein R₅═H, R₁ is an alkyl or cycloalkyl), may be prepared according to SCHEME 2 providing at least one non-limiting synthetic route for the preparation of examples 40 and 42.

Intermediate V (wherein R₁ is H) may be converted into intermediate XIII by an electrophilic halogenation with the corresponding NXS (N-halo succinimide, X: Cl, Br or I) carried out in an organic solvent such as MeCN and at a temperature around RT for a few hours.

Intermediate XIII can be converted into intermediate V (wherein R₁ is alkyl or cycloalkyl) by introducing an R₁ group by means of a metal catalyzed cross coupling such as a palladium catalyzed Suzuki cross coupling, or others described in the reference hereinafter (Strategic application of named reactions in organic synthesis, L. Kurti, B. Czako, Ed. 2005). For example, a Suzuki coupling for inserting an R₁ can be executed by reacting intermediate XIII and a suitable boronic acid or pinacolate derivative in a mixture of water/organic solvent such as DMF or THF, in the presence of a Pd catalyst such as PdCl₂(dppf)₂ DCM adduct or PdXPhos G2, with an inorganic base such as an alkaline carbonate or phosphate, at a temperature around 80° C.-100° C. or higher for a few hours. Intermediate V (wherein R₁ is alkyl or cycloalkyl) can be converted into intermediate X (wherein R₁ is alkyl or cycloalkyl) in a twostep process that includes removal of PG₂ and an amide coupling using the same reactions already described for converting intermediate V into intermediate VI (removal of PG₂) and then intermediate VI into intermediate X (removal of PG₂) of SCHEME 1.

In a different approach intermediate X (wherein R₁ is alkyl or cycloalkyl) may be obtained from intermediate XIV by metal catalyzed cross coupling such as Suzuki as described before for conversion of intermediate XIII into intermediate V (wherein R₁ is alkyl or cycloalkyl). Intermediate XIV may be obtained from intermediate X (wherein R₁ is H) by halogenation, in a similar way to that already described above for conversion of V (wherein R₁ is H) into XIII.

Conversion of intermediate X (wherein R₁ is alkyl or cycloalkyl) into a compound of formula I (wherein R₅═H, R₁ is an alkyl or cycloalkyl) may be carried out as already described in SCHEME 1.

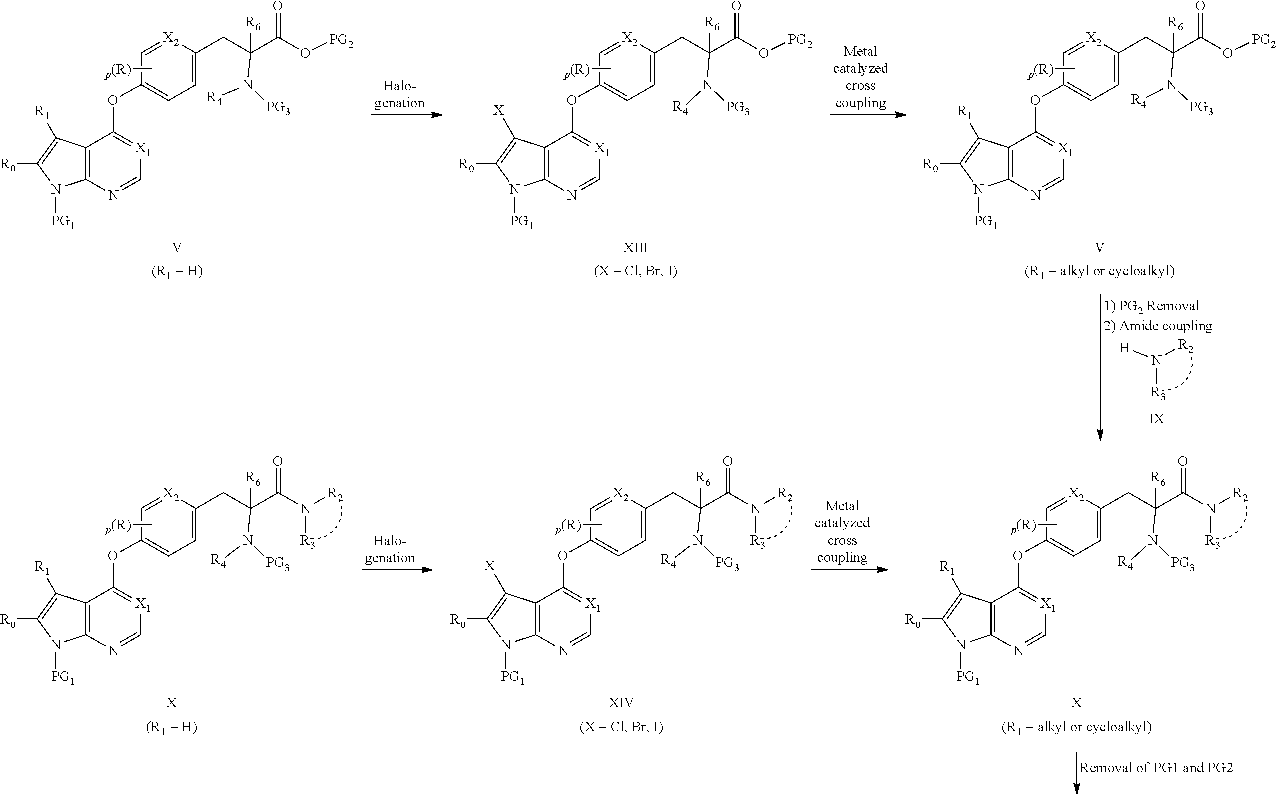

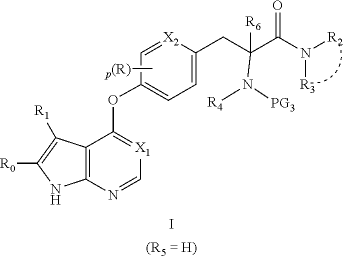

The compounds of the invention are inhibitors of kinase activity, in particular Rho-kinase activity. Generally speaking, compounds which are ROCK inhibitors may be useful in the treatment of many disorders associated with ROCK enzymes mechanisms.

In one embodiment, the disorders that can be treated by the compounds of the present invention include glaucoma, inflammatory bowel disease (IBD) and pulmonary diseases selected from asthma, chronic obstructive pulmonary disease (COPD), interstitial lung disease such as idiopathic pulmonary fibrosis (IPF) and pulmonary arterial hypertension (PAH).

In another embodiment, the disorder that can be treated by the compound of the present invention is selected from the group consisting of asthma, chronic obstructive pulmonary disease (COPD) and interstitial lung disease such as idiopathic pulmonary fibrosis (IPF) and pulmonary arterial hypertension (PAH).

In a further embodiment, the disorder is selected from idiopathic pulmonary fibrosis (IPF) and pulmonary arterial hypertension (PAH).

The methods of treatment of the invention comprise administering a safe and effective amount of a compound of formula (I) or a pharmaceutically acceptable salt thereof to a patient in need thereof. As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan. The compounds of formula (I) or pharmaceutically acceptable salts thereof may be administered once or according to a dosing regimen wherein a number of doses are administered at varying intervals of time for a given period of time. Typical daily dosages may vary depending upon the particular route of administration chosen.

The invention also provides pharmaceutical compositions of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion), by inhalation, rectally, vaginally, topically, locally, transdermally, and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like. Time release capsules, tablets and gels are also advantageous.

Various liquid oral dosage forms can also be used for administering compounds of the invention, including aqueous and non-aqueous solutions, emulsions, suspensions, syrups, and elixirs. Such dosage forms can also contain suitable known inert diluents such as water and suitable known excipients such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the invention can be prepared by mixing the compound with a suitable excipient such as cocoa butter, salicylates and polyethylene glycols.

Formulations for vaginal administration can be in the form of cream, gel, paste, foam, or spray formula containing, in addition to the active ingredient, such as suitable carriers, are also known.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

For the treatment of the diseases of the respiratory tract, the compounds according to the invention are preferably administered by inhalation.

Inhalable preparations include inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier, usually non-toxic and chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers such as Respimat®.

The compounds of the invention can be administered as the sole active agent or in combination (i.e. as co-therapeutic agents administered in fixed dose combination or in combined therapy of separately formulated active ingredients) with other pharmaceutical active ingredients selected from organic nitrates and NO donors; inhaled NO; stimulator of soluble guanylate cyclase (sGC); prostaciclin analogue PGI2 and agonist of prostacyclin receptors; compounds that inhibit the degradation of cyclic guanosine monophosphate (cGMP) and/or cyclic adenosine monophosphate (cAMP), such as inhibitors of phosphodiesterases (PDE) 1, 2, 3, 4 and/or 5, especially PDE 5 inhibitors; human neutrophilic elastase inhibitors; compounds inhibiting the signal transduction cascade, such as tyrosine kinase and/or serine/threonine kinase inhibitors; antithrombotic agents, for example platelet aggregation inhibitors, anticoagulants or profibrinolytic substances; active substances for lowering blood pressure, for example calcium antagonists, angiotensin II antagonists, ACE inhibitors, endothelin antagonists, renin inhibitors, aldosterone synthase inhibitors, alpha receptor blockers, beta receptor blockers, mineralocorticoid receptor antagonists; neutral endopeptidase inhibitor; osmotic agents; ENaC blockers; anti-inflammatories including corticosteroids and antagonists of chemokine receptors; antihistamine drugs; anti-tussive drugs; antibiotics such as macrolide and DNase drug substance and selective cleavage agents such as recombinant human deoxyribonuclease I (rhDNase); agents that inhibit ALK5 and/or ALK4 phosphorylation of Smad2 and Smad3; tryptophan hydroylase 1 (TPH1) inhibitors and multi-kinase inhibitors.

In a preferred embodiment, the compounds of the invention are dosed in combination with phosphodiesterase V such as sildenafil, vardenafil and tadalafil; organic nitrates and NO donors (for example sodium nitroprusside, nitroglycerin, isosorbide mononitrate, isosorbide dinitrate, molsidomine or SIN-1, and inhaled NO); synthetic prostaciclin analogue PGI2 such as iloprost, treprostinil, epoprostenol and beraprost; agonist of prostacyclin receptors such as selexipag and compounds of WO 2012/007539; stimulator of soluble guanylate cyclase (sGC) like riociguat and tyrosine kinase like imatinib, sorafenib and nilotinib and endothelin antagonist (for example macitentan, bosentan, sitaxentan and ambrisentan).

The dosages of the compounds of the invention depend upon a variety of factors including the particular disease to be treated, the severity of the symptoms, the route of administration, the frequency of the dosage interval, the particular compound utilized, the efficacy, toxicology profile, and pharmacokinetic profile of the compound.

Advantageously, the compounds of formula (I) can be administered for example, at a dosage comprised between 0.001 and 1000 mg/day, preferably between 0.1 and 500 mg/day.

When the compounds of formula (I) are administered by inhalation route, they are preferably given at a dosage comprised between 0.001 and 500 mg/day, preferably between 0.1 and 100 mg/day.

A pharmaceutical composition comprising a compound of the invention suitable to be administered by inhalation, such as inhalable powders, propellant-containing metering aerosols or propellant-free inhalable formulations.

The invention is also directed to a device comprising the pharmaceutical composition comprising a compound according to the invention, which may be a single- or multi-dose dry powder inhaler, a metered dose inhaler and a soft mist nebulizer.

The following examples illustrate the invention in more detail.

### PREPARATIONS OF INTERMEDIATES AND EXAMPLES

### General Experimental Details

Purification by chromatography refers to purification using a CombiFlash® Companion purification system or a Biotage SP1 purification system. Where products were purified using an Si cartridge, this refers to an Isolute® pre-packed polypropylene column containing unbounded activated silica with irregular particles with average size of 50 μm and nominal 60 Å porosity. Fractions containing the required product (identified by TLC and/or LCMS analysis) were pooled and concentrated in vacuo. Where an SCX-2 cartridge was used, ‘SCX-2 cartridge’ refers to an Isolute® pre-packed polypropylene column containing a non-end-capped propylsulphonic acid functionalised silica strong cation exchange sorbent. Where HPLC was used for purification (purification by MDAP) fractions containing the required product (identified by TLC and/or LCMS analysis) were pooled and the solvent removed using a Biotage EV10 Evaporator. Alternatively the pooled product fraction was lyophilised.

NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 300 spectrometer with a standard 5 mm dual frequency probe operating at 300 MHz or on a Bruker Fourier 300 spectrometer with a 5 mm dual probe operating at 300 MHz or on Bruker AVANCE III HD 600 spectrometer with a 5 mm probe operating at 600 Mhz. Shifts are given in ppm relative to tetramethylsilane.

LCMS Method 1

Acquity UPLC (binary pump/PDA detector)+ZQ Mass Spectrometer with a C18-reverse-phase column (ACQUITY UPLC BEH C18 1.7 μm, 100×2.1 mm) maintained at 40° C., elution with A: water+0.1% formic acid; B: MeCN+0.1% formic acid.

Gradient:

| Gradient - Time | flow (mL/min) | % A | % B |
|---|---|---|---|
| 0.00 | 0.4 | 95 | 5 |
| 0.40 | 0.4 | 95 | 5 |
| 6.00 | 0.4 | 5 | 95 |
| 6.80 | 0.4 | 5 | 95 |
| 7.00 | 0.4 | 95 | 5 |
| 8.00 | 0.4 | 95 | 5 |

Detection—MS, UV PDA

MS ionisation method—Electrospray (positive/negative ion).

### LCMS Method 2

Acquity i-Class (quaternary pump/PDA detector)+Quattro Micro Mass Spectrometer with a C18-reverse-phase column (ACQUITY UPLC BEH C18 1.7 μm, 100×2.1 mm) maintained at 40° C., elution with A: water+0.1% formic acid; B: MeCN+0.1% formic acid.

Gradient:

| Gradient - Time | flow (mL/min) | % A | % B |
|---|---|---|---|
| 0.00 | 0.4 | 95 | 5 |
| 0.40 | 0.4 | 95 | 5 |
| 6.00 | 0.4 | 5 | 95 |
| 6.80 | 0.4 | 5 | 95 |
| 7.00 | 0.4 | 95 | 5 |
| 8.00 | 0.4 | 95 | 5 |

Detection—MS, UV PDA

MS ionisation method—Electrospray (positive/negative ion).

### LCMS Method 3

Acquity H-Class (quaternary pump/PDA detector)+QDa Mass Spectrometer with a C18-reverse-phase column (Acquity UPLC CSH C18 1.7 μm, 50×2.1 mm) maintained at 40° C., elution with A: water+0.1% formic acid; B: MeCN+0.1% formic acid.

Gradient:

| Gradient - Time | flow (mL/min) | % A | % B |
|---|---|---|---|
| 0.00 | 1.0 | 97 | 3 |
| 4.00 | 1.0 | 1 | 99 |
| 4.4 | 1.0 | 1 | 99 |
| 4.5 | 1.0 | 97 | 3 |
| 5.0 | 1.0 | 97 | 3 |

Detection—MS, UV PDA

MS ionisation method—Electrospray (positive/negative ion).

### LCMS Method 4

UPLC+Waters DAD+Waters SQD2, single quadrupole UPLC-MS with a C18-reverse-phase column (Acquity UPLC BEH Shield RP18 1.7 μm 100×2.1 mm), elution with A: water with 10 mM ammonium bicarbonate (ammonium hydrogen carbonate); B: MeCN.

Gradient:

| Gradient - Time | flow (mL/min) | % A | % B |
|---|---|---|---|
| 0.0 | 0.5 | 95 | 5 |
| 1.2 | 0.5 | 95 | 5 |
| 3.5 | 0.5 | 0 | 100 |
| 4.9 | 0.5 | 0 | 100 |
| 5.0 | 0.5 | 95 | 5 |
| 6.0 | 0.5 | 95 | 5 |

Detection—MS, UV PDA

MS ionisation method—Electrospray (positive/negative ion).

LCMS Method 5 UPLC+Waters DAD+Waters SQD2, single quadrupole UPLC-MS with a C18-reverse-phase column (Acquity UPLC BEH Shield RP18 1.7 μm 100×2.1 mm), elution with A: water with 10 mM ammonium bicarbonate (ammonium hydrogen carbonate); B: MeCN.

Gradient:

| Gradient - Time | flow (mL/min) | % A | % B |
|---|---|---|---|
| 0.0 | 0.4 | 95 | 5 |
| 0.4 | 0.4 | 95 | 5 |
| 6.0 | 0.4 | 5 | 95 |
| 6.8 | 0.4 | 5 | 95 |
| 7.0 | 0.4 | 95 | 5 |
| 8.0 | 0.4 | 95 | 5 |

Detection—MS, UV PDA

MS ionisation method—Electrospray (positive/negative ion).

### LCMS Method 6

UPLC+Waters DAD+Waters SQD2, single quadrupole UPLC-MS with a C18-reverse-phase column (Acquity UPLC HSS C18 1.8 μm 100×2.1 mm), elution with A: water with 0.1% formic acid; B: MeCN with 0.1% formic acid.

Gradient:

| Gradient - Time | flow (mL/min) | % A | % B |
|---|---|---|---|
| 0.0 | 0.5 | 95 | 5 |
| 1.2 | 0.5 | 95 | 5 |
| 3.5 | 0.5 | 0 | 100 |
| 4.9 | 0.5 | 0 | 100 |
| 5.0 | 0.5 | 95 | 5 |
| 6.0 | 0.5 | 95 | 5 |

Detection—MS, UV PDA

MS ionisation method—Electrospray (positive/negative ion).

### LCMS Method 7

Acquity H-Class (quaternary pump/PDA detector)+QDa Mass Spectrometer with a C18-reverse-phase column (Acquity BEH 1.7 μm, 50×2.1 mm) maintained at 50° C., elution with A: water+0.10% formic acid; B: MeCN+0.10% formic acid.

Gradient:

| Gradient - Time | flow (mL/min) | % A | % B |
|---|---|---|---|
| 0.00 | 1 | 97 | 3 |
| 1.50 | 1 | 1 | 99 |
| 1.90 | 1 | 1 | 99 |
| 2.00 | 1 | 97 | 3 |
| 2.50 | 1 | 97 | 3 |

Detection—MS, UV PDA

MS ionisation method—Electrospray (positive/negative ion).

### LCMS Method 8

UPLC+Waters DAD+Waters SQD2, single quadrupole UPLC-MS with a C18 reverse-phase column (Acquity UPLC HSS C18 1.8 μm 100×2.1 mm), elution with A: water with 0.1% formic acid; B: MeCN with 0.1% formic acid.

Gradient:

| Gradient - Time | flow (mL/min) | % A | % B |
|---|---|---|---|
| 0.00 | 0.4 | 95 | 5 |
| 0.40 | 0.4 | 95 | 5 |
| 6.00 | 0.4 | 5 | 95 |
| 6.80 | 0.4 | 5 | 95 |
| 7.0 | 0.4 | 95 | 5 |
| 8.0 | 0.4 | 95 | 5 |

Detection—MS, UV PDA

MS ionisation method—Electrospray (positive/negative ion).

### LCMS Method 9

Acquity H-Class (quaternary pump/PDA detector)+QDa Mass Spectrometer with a C18-reverse-phase column (Acquity UPLC CSH C18 1.7 μm, 50×2.1 mm) maintained at 40° C., elution with A: water+0.1% formic acid; B: MeCN+0.1% formic acid.

Gradient:

| Gradient - Time | flow (mL/min) | % A | % B |
|---|---|---|---|
| 0.0 | 1.0 | 97 | 3 |
| 1.5 | 1.0 | 1 | 99 |
| 1.9 | 1.0 | 1 | 99 |
| 2.0 | 1.0 | 97 | 3 |
| 2.5 | 1.0 | 97 | 3 |

Detection—MS, UV PDA

MS ionisation method—Electrospray (positive/negative ion).

### MDAP Method

Agilent Technologies 1260 Infinity purification system with column maintained at RT and a flow rate of 20 ml/min. The column, eluent and gradient are specified within individual experimental descriptions.

### SFC Methods

Supercritical Fluid Chromatography (SFC) was carried out using either a Waters Thar Prep100 preparative SFC system (P200 CO₂ pump, 2545 modifier pump, 2998 UV/VIS detector, 2767 liquid handler with Stacked Injection Module) or a Waters Thar Investigator semi preparative system (Waters Fluid Delivery Module, 2998 UV/VIS detector, Waters Fraction Collection Module). The column and isocratic method used is indicated for each compound and the single enantiomers were analysed using the methods given. Some of the compounds may have gone through a second purification process in order to achieve the required % ee purity.

### Abbreviations Used:

Boc=tert-butoxycarbonyl; COMU (1-Cyano-2-ethoxy-2-oxoethylidenaminooxy)-dimethylamino-morpholinocarbenium hexafluorophosphate; DCE=1,2-Dichloroethane; DCM=Dichloromethane; DEA=Diethylamine; DIPEA=Di-isopropylethylamine; DMF=N,N-dimethylformamide; DMSO=Dimethylsulphoxide; h=Hour(s); HATU=(1-[Bis(dimethylamino)methylene]-1H-1,2,3-triazolo[4,5-b]pyridinium 3-oxide hexafluorophosphate); HPLC=High performance liquid chromatography; IMS=Industrial methylated spirits; LCMS=Liquid chromatography-mass spectrometry; MDAP=Mass-directed autopurification; MeCN=Acetonitrile; NIS=N-Iodosuccinimide; Pd₂(dba)₃=Tris(dibenzylideneacetone)dipalladium(0); Pd(dppf)Cl₂.DCM=Bis(diphenylphosphino)ferrocene]dichloropalladium(II), complex with dichloromethane; Pd Xphos G2=Chloro(2-dicyclohexylphosphino-2′,4′,6′-triisopropyl-1,1′-biphenyl)[2-(2′-amino-1,1′-biphenyl)]palladium(II); Rt=Retention time; RT=Room temperature; SCX=Strong cation exchange; SFC=Supercritical Fluid Chromatography; TEA=Triethylamine; TFA=Trifluoroacetic acid; THF=Tetrahydrofuran; XPhos=2-Dicyclohexylphosphino-2′,4′,6′-triisopropylbiphenyl.

In the procedures that follow, some of the starting materials are identified through an “Intermediate” or “Example” number with indications on step number. This is provided merely for assistance to the skilled chemist.

A “similar” or “analogous” procedure means that such a procedure may involve minor variations, for example reaction temperature, reagent/solvent amount, reaction time, work-up conditions or chromatographic purification conditions.

The stereochemistry of the compounds in the Examples, where indicated, has been assigned on the assumption that absolute configuration at resolved stereogenic centers of starting materials is maintained throughout any subsequent reaction conditions.

The ee % (enantiomeric excess) was measured by readily available chiral LC or SFC methods, for example as reported for Examples 8. This method is to be considered as an examples of an analytical method to be used for the determination of ee %.

Unless otherwise stated, where absolute configuration (R) or (S) is reported in the compound name, ee % has to be considered equal or greater than 90%. For those Examples having a measured value of ee % less than 90, the exact value was reported. Wherein the measure of ee % has not been determined, they were marked as n.d. (not determined).

### Example 1

Step A

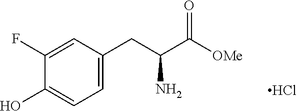 [SMILES: COC(=O)[C@@H](N)Cc1ccc(O)c(F)c1 ; Cl]

### Methyl (S)-2-amino-3-(3-fluoro-4-hydroxyphenyl)propanoate hydrochloride (Intermediate 1A-a)

Thionyl chloride (36.6 mL, 0.5 mol) was added dropwise to chilled methanol (200 mL) at 0° C. with stirring. The mixture was stirred cold at that temperature for 15 min then 3-fluoro-L-tyrosine (20 g, 100 mmol) was added portionwise. The resulting solution was allowed to warm to RT and stirred for 18 h. The mixture was concentrated in vacuo to afford Intermediate 1A-a as solid (25.2 g).

LCMS (Method 9): Rt=0.16 min and 0.25 min, m/z 214.1 [M+H]⁺

Step B

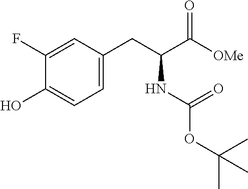 [SMILES: COC(=O)[C@H](Cc1ccc(O)c(F)c1)NC(=O)OC(C)(C)C]

### Methyl (S)-2-((tert-butoxycarbonyl)amino)-3-(3-fluoro-4-hydroxyphenyl)-propanoate (Intermediate 1B-a)

Saturated sodium bicarbonate solution (aq.) (200 mL) was added to a vigorously stirred suspension of Intermediate 1A-a (25.2 g, 100 mmol) in THF (200 mL). The mixture was stirred until gas evolution ceased then a solution of di-tert-butyl dicarbonate (25.55 g, 117 mmol) in THF (20 mL) was added in one portion. The mixture was stirred until obvious gas evolution ceased then for a further 1.25 h. The mixture was partitioned between water (200 mL) and ethyl acetate (200 mL). The organic phase was washed with water (100 mL) and the combined aqueous phase was then washed with ethyl acetate (100 mL). The combined organic phase was washed with brine, dried (sodium sulfate) and concentrated in vacuo to give Intermediate 1B-a (34.2 g).

LCMS (Method 9): Rt=0.81 min, m/z 312.1 [M−H]⁻

Step C

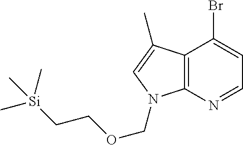 [SMILES: Cc1cn(COCC[Si](C)(C)C)c2nccc(Br)c12]

### 4-Bromo-3-methyl-1-((2-(trimethylsilyl)ethoxy)methyl)-1H-pyrrolo[2,3-b]pyridine (Intermediate 1C-a)

To a chilled (ice/water bath) suspension of sodium hydride (3.41 g of a 60% dispersion in mineral oil, 85 mmol) in acetonitrile (200 mL) under a stream of nitrogen was added, portion-wise, 4-bromo-3-methyl-1H-pyrrolo[2,3-b]pyridine (20 g, 94.8 mmol). The mixture was stirred cold until gas evolution ceased. A solution of 2-(trimethylsilyl)ethoxymethyl chloride (20.1 mL, 114 mmol) in acetonitrile (20 mL) was added slowly. The chilled mixture was stirred for 2 h (temperature maintained below 15° C.) then diluted with ethyl acetate (200 mL). Water (200 mL) was added cautiously. The phases were separated. The organic phase was washed with water (2×100 mL) then brine (100 mL) then dried (Na₂SO₄) and concentrated in vacuo. The residue was chromatographed on a silica pad eluting with 0-10% ethyl acetate in cyclohexane. Concentration of appropriate fractions gave Intermediate 1C-a (28.3 g).

LCMS (Method 7): Rt=1.76 min, m/z 341.1/343.0 [M+H]⁺

Step D

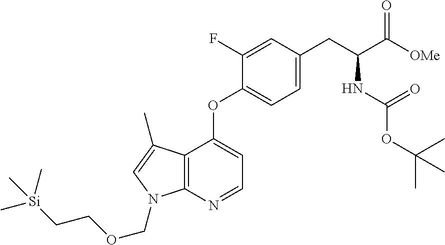 [SMILES: COC(=O)[C@H](Cc1ccc(Oc2ccnc3c2c(C)cn3COCC[Si](C)(C)C)c(F)c1)NC(=O)OC(C)(C)C]

### Methyl (S)-2-((tert-butoxycarbonyl)amino)-3-(3-fluoro-4-((3-methyl-1-((2-(trimethylsilyl)ethoxy)methyl)-1H-pyrrolo[2,3-b]pyridin-4-yl)oxy)phenyl)-propanoate (Intermediate 1D-a)

A mixture of Intermediates 1B-a (52.0 g, 166 mmol) and 1C-a (59.5 g, 174 mmol), Pd₂(dba)₃ (7.6 g, 8.3 mmol), XPhos (7.91 g, 17 mmol), and potassium carbonate (49.3 g, 357 mmol) in toluene (600 mL) was purged with argon for 5 min. The mixture was heated under argon at 100° C. for 5 h, and then allowed to cool to RT before filtering through Celite®. The solvent was evaporated, the residue was diluted with ethyl acetate, and the organic layer was washed three times with water. The combined aqueous layers were extracted with ethyl acetate and the combined organic extracts were washed with brine, dried (Na₂SO₄) and evaporated. The crude product was chromatographed on a silica pad eluting with 10-25% ethyl acetate in isohexane to give the title compound (69.5 g).

LCMS (Method 9): Rt=1.88 min, m/z 574.4 [M+H]⁺

Step E

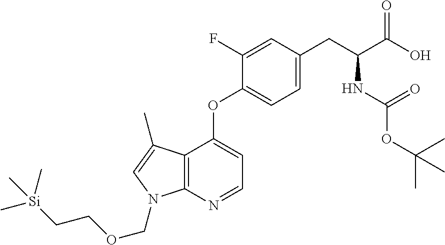 [SMILES: Cc1cn(COCC[Si](C)(C)C)c2nccc(Oc3ccc(C[C@H](NC(=O)OC(C)(C)C)C(=O)O)cc3F)c12]

### (S)-2-((tert-Butoxycarbonyl)amino)-3-(3-fluoro-4-((3-methyl-1-((2-(trimethylsilyl)ethoxy)methyl)-1H-pyrrolo[2,3-b]pyridin-4-yl)oxy)phenyl)propanoic Acid Lithium Salt (Intermediate 1E-a)

Intermediate 1D-a (4.85 g, 8.45 mmol) was dissolved in a mixture of methanol (42 mL), water (42 mL) and THF (21 mL). Lithium hydroxide hydrate (1.06 g, 25.35 mmol) was added and the reaction mixture was stirred at RT for 10 min. The solvent was reduced and the product was extracted into ethyl acetate (3×20 mL). The combined organic extracts were washed with brine (30 mL), dried (Na₂SO₄) and evaporated to give the title compound (4.74 g).

LCMS (Method 7): Rt=1.79 min, m/z 560.4 [M+H]⁺

Step F

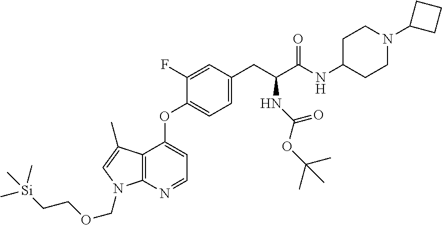 [SMILES: Cc1cn(COCC[Si](C)(C)C)c2nccc(Oc3ccc(C[C@H](NC(=O)OC(C)(C)C)C(=O)NC4CCN(C5CCC5)CC4)cc3F)c12]

### tert-Butyl (S)-(1-((1-cyclobutylpiperidin-4-yl)amino)-3-(3-fluoro-4-((3-methyl-1-((2-(trimethylsilyl)ethoxy)methyl)-1H-pyrrolo[2,3-b]pyridin-4-yl)oxy)phenyl)-1-oxopropan-2-yl)carbamate (Intermediate 1F-a)

To a mixture of Intermediate 1E-a (0.2 g, 0.37 mmol), 1-cyclobutylpiperidin-4-amine (0.066 g, 0.43 mmol), and DIPEA (0.19 mL, 1.07 mmol) in DCM (10 mL) was added COMU (0.18 g, 0.43 mmol) and the reaction was stirred at RT for 2 h. before being concentrated in vacuo. The residue was partitioned between ethyl acetate (3×30 mL) and saturated aqueous NaHCO₃ (20 mL). The organic layer was washed with brine (20 mL) dried (Na₂SO₄) and evaporated in vacuo to afford the desired product that was used in the next step without further purification.

LCMS (Method 9): Rt=1.20 min, m/z 696.5 [M+H]⁺

Step G

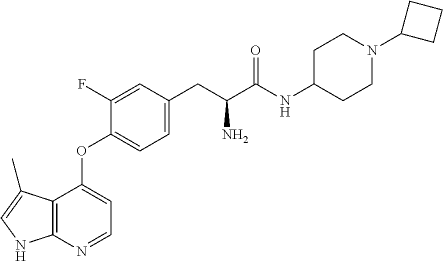 [SMILES: Cc1c[nH]c2nccc(Oc3ccc(C[C@H](N)C(=O)NC4CCN(C5CCC5)CC4)cc3F)c12]

### (S)-2-Amino-N-(1-cyclobutylpiperidin-4-yl)-3-(3-fluoro-4-((3-methyl-1H-pyrrolo[2,3-b]pyridin-4-yl)oxy)phenyl)propanamide (Example 1)

Intermediate 1F-a (0.28 g, 0.403 mmol) was dissolved in a mixture of DCM (10 mL) and TFA (10 mL), and the reaction was stirred at RT for 1 h. The mixture was passed down a 20 g SCX-2 cartridge eluting with DCM, methanol and then 2M methanolic ammonia. After standing for 18 h, the ammonia solution was evaporated to give a pale yellow residue which was purified by MDAP using an Xbridge Phenyl column (19×150 mm, 10 μm particle size) and eluting with 40-100% MeOH/H₂O (10 mM NH₄CO₃) to give the title compound (111 mg).

LCMS (Method 1): Rt=1.95 min, m/z 466.3[M+H]⁺

¹H NMR (400 MHz, d6-DMSO) δ 11.4 (s, 1H), 7.97 (d J=5.45, 1H), 7.64 (d J=7.94, 1H), 7.30-7.17 (m, 2H), 7.15-7.04 (m, 1H), 6.15 (d J=5.48 Hz, 1H), 3.51-3.41 (m, 1H), 2.91-2.79 (m, 1H), 2.76-2.55 (m, 4H), 2.38 (s, 3H), 1.99-1.86 (m, 2H), 1.65-1.48 (m, 4H), 1.38-1.15 (m, 2H).

### Preparation of Intermediates 1C-a and 1C-b

The following intermediates were prepared in a similar manner to Intermediate 1C-a from the indicated starting materials.

| Intermediate | Structure | Starting materials | LC-MS |
|---|---|---|---|
| 1C-b | 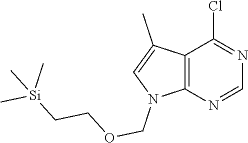 [SMILES: Cc1cn(COCC[Si](C)(C)C)c2ncnc(Cl)c12] | 4-Chloro-5-methyl-7H- pyrrolo[2,3-d]pyrimidine | Rt = 2.35 min, m/z 298.1 [M + H]⁺ (Method 9) |
| 1C-c | 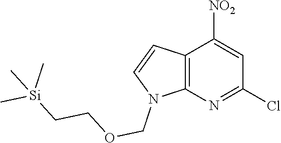 [SMILES: C[Si](C)(C)CCOCn1ccc2c([N+](=O)[O-])cc(Cl)nc21] | 6-Chloro-4-nitro-1H- pyrrolo[2,3-b]pyridine | Rt = 1.76 min, m/z 328.1 [M + H]⁺ (Method 3) |

### Preparation of Intermediate 1D-b

The following intermediate was prepared in a similar manner to Intermediate 1D-a from the indicated starting materials.

| Intermediate | Structure | Starting materials | LC-MS |
|---|---|---|---|
| 1D-b | 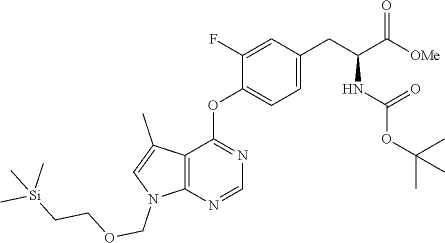 [SMILES: COC(=O)[C@H](Cc1ccc(Oc2ncnc3c2c(C)cn3COCC[Si](C)(C)C)c(F)c1)NC(=O)OC(C)(C)C] | 1B-a and 1C-b | Rt = 2.48 min, m/z 575.3 [M + H]⁺ (Method 7) |

### Preparation of Intermediate 1E-b

The following intermediate was prepared in a similar manner to Intermediate 1E-a from the indicated starting materials.

| Intermediate | Structure | Starting materials | LC-MS |
|---|---|---|---|
| 1E-b | 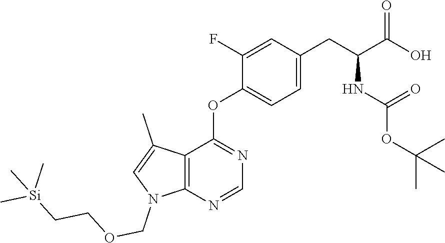 [SMILES: Cc1cn(COCC[Si](C)(C)C)c2ncnc(Oc3ccc(C[C@H](NC(=O)OC(C)(C)C)C(=O)O)cc3F)c12] | 1D-b | Rt = 1.71 min, m/z 561.3 [M + H]⁺ (Method 3) |

### Intermediate 19C

Step A

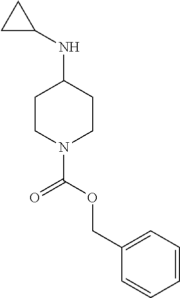 [SMILES: O=C(OCc1ccccc1)N1CCC(NC2CC2)CC1]

### Benzyl 4-(cyclopropylamino)piperidine-1-carboxylate (Intermediate 19A)

Sodium triacetoxyborohydride (1.36 g, 6.43 mmol) was added portionwise to an ice cooled solution of benzyl 4-oxopiperidine-1-carboxylate (1.0 g, 4.29 mmol), cyclopropylamine (0.45 mL, 6.43 mmol) and acetic acid (0.37 mL, 6.43 mmol) in DCM (10 mL). The resulting mixture was allowed to warm to RT and stirred for 18 hours. A further amount of cyclopropylamine (0.15 mL, 2.15 mmol), acetic acid (0.12 mL, 2.15 mmol) and sodium triacetoxyborohydride (0.45 g, 2.15 mmol) were added and the resulting mixture was stirred for 7 days. The reaction was quenched by addition of saturated aqueous solution NaHCO₃ and extracted with DCM (×3). The organic phase was dried (Na₂SO₄), filtered and concentrated under reduced pressure. Purification on a 40 g Si cartridge eluting with 0-5% 7N methanolic ammonia in DCM afforded the desired product (385 mg).

LCMS (Method 9): Rt=0.74 min, m/z 275.1 [M+H]⁺

Step B

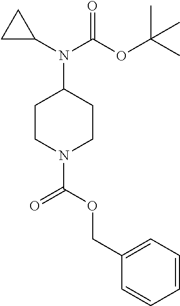 [SMILES: CC(C)(C)OC(=O)N(C1CC1)C1CCN(C(=O)OCc2ccccc2)CC1]

### Benzyl 4-((tert-butoxycarbonyl)(cyclopropyl)amino)piperidine-1-carboxylate (Intermediate 19B)

Intermediate 19A (385 mg, 1.40 mmol) was dissolved in THF (4.0 mL), treated with aqueous 2M sodium carbonate solution (1.2 mL, 2.46 mmol) followed by di-tert-butyl dicarbonate (368 mg, 1.68 mmol) and the resulting mixture was stirred at RT for 72 hours. The mixture was diluted with water and extracted with EtOAc (×3). The organic phase was dried (Na₂SO₄), filtered and concentrated under reduced pressure. Purification on a 25 g Si cartridge eluting with 0-50% EtOAc in cyclohexane afforded the desired product (454 mg).

LCMS (Method 9): Rt=1.57 min. m/z 275.1 [M+H-Boc]⁺

Step C

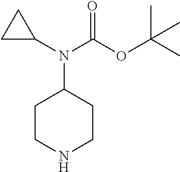 [SMILES: CC(C)(C)OC(=O)N(C1CCNCC1)C1CC1]

### tert-butyl cyclopropyl(piperidin-4-yl)carbamate (Intermediate 19C)

Intermediate 19B (454 mg, 1.21 mmol) was dissolved in IMS (10.0 mL), treated with 10% palladium on carbon (50 mg) and the resulting mixture was stirred under hydrogen for 18 hours. The reaction mixture was filtered through Celite® and the filtrate concentrated under reduced pressure to afford the desired product (264 mg).

LCMS (Method 9): Rt=0.74 min, m/z 241.1 [M+H]⁺

### Intermediate 20D

Step A

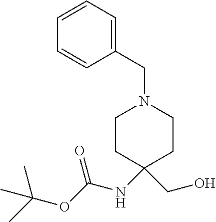 [SMILES: CC(C)(C)OC(=O)NC1(CO)CCN(Cc2ccccc2)CC1]

### tert-Butyl (1-benzyl-4-(hydroxymethyl)piperidin-4-yl)carbamate (Intermediate 20A)

(4-Amino-1-benzylpiperidin-4-yl)methanol (500 mg, 1.89 mmol) was dissolved in DCM (8.0 mL) and the resulting mixture was cooled with an ice bath. Then Boc anhydride (475 mg, 2.18 mmol) was added in one portion followed by the dropwise addition of TEA (0.26 mL). The resulting suspension was allowed to stir at RT overnight. The reaction was washed with saturated aqueous solution NaHCO₃ (5 mL). The organic phase was dried (Na₂SO₄), filtered and concentrated under reduced pressure to afford a colourless oil. Purification on a Si cartridge eluting with 0-10% DCM in MeOH afforded the desire product (580 mg).

¹H NMR (400 MHz, CDCl₃) δ 7.36-7.35 (m, 5H), 5.14-5.12 (m, 2H), 4.52 (s, 1H), 3.79 (s, 2H), 3.70 (d, J=6.1 Hz, 2H), 3.62-3.49 (m, 1H), 3.28-3.20 (m, 2H), 1.89 (d, J=13.4 Hz, 2H), 1.68-1.57 (m, 2H), 1.45 (s, 9H).

Step B

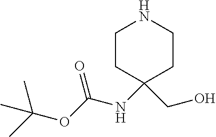 [SMILES: CC(C)(C)OC(=O)NC1(CO)CCNCC1]

### tert-Butyl (4-(hydroxymethyl)piperidin-4-yl)carbamate (Intermediate 20B)

Intermediate 20A (580 mg, 1.59 mmol) and palladium (10%) (169 mg, 0.10 mmol) were suspended in IMS (4.0 mL) then the reaction mixture was exposed to a hydrogen atmosphere via a balloon. The reaction mixture was allowed at stir at RT overnight, then the reaction mixture was flushed through a pad of Celite® and dried to afford a pale yellow oil. The residue was carried onto the next step without purification (367 mg).

¹H NMR (400 MHz, CDCl₃) δ, 4.61 (s, 1H), 2.96-2.78 (m, 4H), 2.73-2.21 (m, 2H), 1.87 (d, J=13.9 Hz, 2H), 1.70-1.59 (m, 2H), 1.44 (s, 9H).

Step C

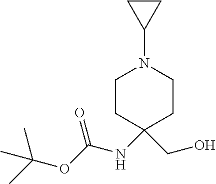 [SMILES: CC(C)(C)OC(=O)NC1(CO)CCN(C2CC2)CC1]

### tert-Butyl (1-cyclopropyl-4-(hydroxymethyl)piperidin-4-yl)carbamate (Intermediate 20C)

Intermediate 20B (281 mg, 1.22 mmol) was dissolved in methanol (5.0 mL), then (1-ethoxycyclopropylpropoxy)trimethylsilane (0.74 mL, 3.66 mmol) was added dropwise and sodium cyanoborohydride (230 mg, 3.66 mmol) was added in one portion. The resulting mixture was allowed to stir at 60° C. overnight. The reaction mixture was allowed to cool to RT and was passed through a pad of Celite® eluting with methanol. The solution was concentrated, re-dissolved in ethyl acetate (5 mL) and washed with 1M NaOH (5 mL). The organic phase was dried (Na₂SO₄), filtered and concentrated to afford the desired product (240 mg).

LCMS (Method 9): Rt=0.19 min, m/z 271.2 [M+H]⁺

Step D

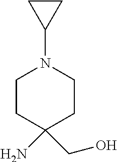 [SMILES: NC1(CO)CCN(C2CC2)CC1]

### (4-Amino-1-cyclopropylpiperidin-4-yl)methanol (Intermediate 20D)

Intermediate 20C (240 mg, 0.888 mmol) was dissolved in DCM (4.0 mL) and TFA (2 mL) was added dropwise. Then the resulting mixture was stirred at RT for 18 h. The reaction mixture was loaded onto a 5 g SCX-2 cartridge eluting with methanol and then 2M methanolic ammonia. The eluent was concentrated to afford the desired product (151 mg).

LCMS (Method 9): Rt=0.17 min, m/z 171.2 [M+H]⁺

### Intermediate 21D

Step A

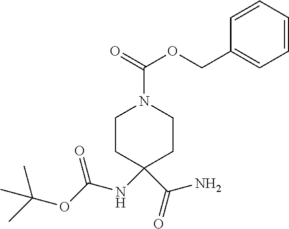 [SMILES: CC(C)(C)OC(=O)NC1(C(N)=O)CCN(C(=O)OCc2ccccc2)CC1]

### Benzyl 4-((tert-butoxycarbonyl)amino)-4-carbamoylpiperidine-1-carboxylate (Intermediate 21A)

1-((Benzyloxy)carbonyl)-4-((tert-butoxycarbonyl)amino)piperidine-4-carboxylic acid (500 mg, 1.32 mmol) and ammonium chloride (141 mg, 2.64 mmol) were stirred in DMF (15 mL) and COMU® (849 mg, 1.98 mmol) and DIPEA (0.92 mL, 5.29 mmol) were added. The reaction mixture was stirred at RT overnight then the mixture was partitioned between water and ethyl acetate. The phases were separated then the organic phase was dried (Na₂SO₄), filtered and concentrated. Purification by flash column chromatography on a 40 g Si cartridge eluting with 0-5% DCM in methanol gave the desired product (422 mg).

LCMS (Method 9): Rt=1.17 min. m/z 400 [M+Na]⁺

Step B

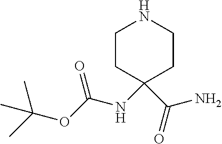 [SMILES: CC(C)(C)OC(=O)NC1(C(N)=O)CCNCC1]

### tert-Butyl (4-carbamoylpiperidin-4-yl)carbamate (Intermediate 21B)

Intermediate 21A (420 mg, 1.11 mmol) and palladium hydroxide on carbon (20%) (42 mg, 0.30 mmol) were suspended in IMS (15 mL) then the reaction mixture was exposed to hydrogen atmosphere via a balloon. The reaction mixture was allowed to stir at room temperature for 72 h and then flushed through a pad of Celite® and dried. The white solid was carried onto the next step without purification (258 mg).

LCMS (Method 9): Rt=0.16 min, m/z 244 [M+H]⁺

Step C

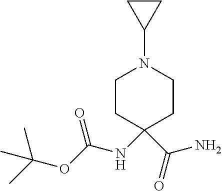 [SMILES: CC(C)(C)OC(=O)NC1(C(N)=O)CCN(C2CC2)CC1]

### tert-Butyl (4-carbamoyl-1-cyclopropylpiperidin-4-yl)carbamate (Intermediate 21C)

Intermediate 21B (258 mg, 1.06 mmol) was dissolved in methanol (5.0 mL), then (1-ethoxycyclopropylpropoxy)trimethylsilane (0.64 mL, 3.18 mmol) was added followed by sodium cyanoborohydride (200 mg, 3.18 mmol). The resulting mixture was allowed to stir at 60° C. overnight. Then the reaction mixture was allowed to cool to RT and concentrated. Flash column chromatography on a 25 g Si cartridge eluting with 0-5% DCM in methanol gave the desired product (101 mg).

¹H NMR (400 MHz, CDCl₃) δ 6.71 (s, 1H), 5.52 (s, 1H), 4.89 (s, 1H), 3.06-1.65 (m, 9H), 1.46 (s, 9H), 0.52 (m, 4H).

Step D

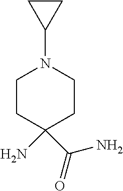 [SMILES: NC(=O)C1(N)CCN(C2CC2)CC1]

### 4-Amino-1-cyclopropylpiperidine-4-carboxamide (Intermediate 21D)

Intermediate 21C (101 mg, 0.356 mmol) was dissolved in DCM (4.0 mL) and TFA (2 mL) was added. The resulting mixture was stirred at RT for 1 h. The reaction mixture was diluted with methanol then loaded onto a methanol-wetted 5 g SCX-2 cartridge eluting with methanol and then 2M methanolic ammonia. The ammonia solution was concentrated to afford the desired product (68 mg).

LCMS (Method 9): Rt=0.24 min, m/z 184.2 [M+H]⁺

### Intermediate 31C

Step A

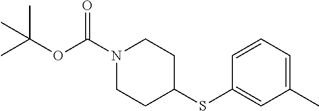 [SMILES: Cc1cccc(SC2CCN(C(=O)OC(C)(C)C)CC2)c1]

### tert-Butyl 4-(m-tolylthio)piperidine-1-carboxylate (Intermediate 31A)

To a solution of di-tert-butyl dicarbonate (0.99 mL, 4.31 mmol), in DCM (30 mL), at 0° C., was added 4-((3-methylphenyl)thiopiperidine hydrochloride (1.00 g, 4.10 mmol), then TEA (1.70 mL, 12.31 mmol). The mixture was stirred for 4 h whilst being allowed to warm to RT. The reaction mixture was evaporated, then purified by flash column chromatography, on an 80 g Si cartridge, eluting with 0-50% EtOAc in cyclohexane to afford the title compound (1.14 g).

LCMS (Method 9): Rt=1.67 min. 208.1 [M-Boc+H]⁺

¹H NMR (300 MHz, CDCl₃) δ 7.26 (s, 1H), 7.25-7.23 (m, 1H), 7.22-7.20 (m, 1H), 7.09-7.04 (m, 1H), 3.96 (d, J=12.7 Hz, 2H), 3.25-3.14 (m, 1H), 2.98-2.86 (m, 2H), 2.33 (s, 3H), 1.96-1.85 (m, 2H), 1.60-1.48 (m, 2H), 1.44 (s, 9H).

Step B

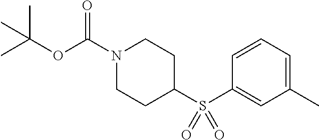 [SMILES: Cc1cccc(S(=O)(=O)C2CCN(C(=O)OC(C)(C)C)CC2)c1]

### tert-Butyl 4-(m-tolylsulfonyl)piperidine-1-carboxylate (Intermediate 31B)

A solution of Intermediate 31A (1.14 g, 3.09 mmol), in DCM (30 mL), was cooled to 0° C. then 3-chloroperbenzoic acid (1.47 g, 8.53 mmol) was added. The mixture was stirred at 0° C. for 10 mins, then allowed to stir at RT overnight. The reaction mixture was quenched by addition of saturated aqueous NaHCO₃ (25 mL) and sodium metabisulfite (916 mg) and stirred. DCM was added and the organics were separated using a phase separator cartridge and evaporated. The crude material was purified by flash column chromatography, on an 80 g Si cartridge, eluting with 0-50% EtOAc in cyclohexane to give the title compound (806 mg).

LCMS (Method 9): Rt=1.38, m/z 240.1 [M-Boc+H]⁺

¹H NMR (300 MHz, CDCl₃) δ 7.69-7.63 (m, 2H), 7.48-7.44 (m, 2H), 4.23 (d, J=12.6 Hz, 2H), 3.08-2.96 (m, 1H), 2.72-2.57 (m, 2H), 2.46 (s, 3H), 1.98 (d, J=12.8 Hz, 2H), 1.69-1.53 (m, 2H), 1.43 (s, 9H).

Step C

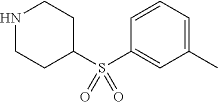 [SMILES: Cc1cccc(S(=O)(=O)C2CCNCC2)c1]

### 4-(m-Tolylsulfonyl)piperidine (Intermediate 31C)

To a solution of Intermediate 31B (800 mg, 2.36 mmol), in DCM (10 mL) under argon, was added TFA (5 mL) and the reaction mixture was stirred at RT for 2.75 h. The mixture was diluted with methanol, then applied to a methanol wetted SCX-2 cartridge (10 g), washed with methanol then eluted using 2 N ammonia in methanol. The ammonia fraction was evaporated to give the title compound (546 mg).

LCMS (Method 9): Rt=0.62 min, m/z 240.1 [M+H]⁺

### Examples 2 to 39

The following examples were prepared in a similar manner to Example 1, following the same synthetic sequence, by replacing in Step F the indicated Intermediate 1E and amine starting materials in the table below.

| | | Intermediate | | |
|---|---|---|---|---|
| Ex | Structure/Name | 1E/Amine | 1H NMR | LC-MS |
| 2 | 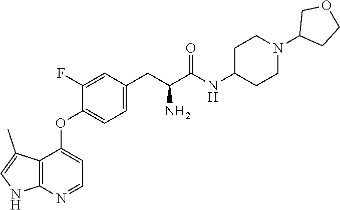 [SMILES: Cc1c[nH]c2nccc(Oc3ccc(C[C@H](N)C(=O)NC4CCN(C5CCOC5)CC4)cc3F)c12] 2 (2S)-2-Amino-3-(3-fluoro-4-((3-methyl-1H- pyrrolo[2,3-b]pyridin-4-yl)oxy)phenyl)-N-(1- (tetrahydrofuran-3-yl)piperidin-4-yl)propanamide | 1E-a/1-(tetrahydro- furan-3- yl)piperidin- 4-amine | ¹H NMR (400 MHz, DMSO) δ 11.40 (s, 1H), 7.97 (d, J = 5.4 Hz, 1H), 7.68-7.64 (m, 1H), 7.28-7.20 (m, 2H), 7.14-7.07 (m, 2H), 6.15 (d, J = 5.4 Hz, 1H), 3.80- 3.70 (m, 2H), 3.65-3.59 (m, 1H), 3.53- 3.37 (m, 4H), 2.89-2.68 (m, 4H), 2.62- 2.56 (m, 1H), 2.39-2.37 (m, 3H), 2.06- 1.89 (m, 4H), 1.72-1.58 (m, 3H), 1.40- 1.24 (m, 2H). | Rt = 1.82 min, m/z 482.4 [M + H]⁺ (Method 1) |
| 3 | 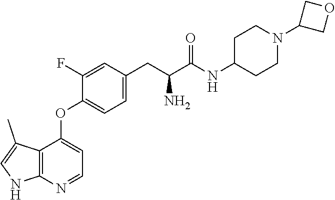 [SMILES: Cc1c[nH]c2nccc(Oc3ccc(C[C@H](N)C(=O)NC4CCN(C5COC5)CC4)cc3F)c12] (S)-2-Amino-3-(3-fluoro-4-((3-methyl-1H- pyrrolo[2,3-b]pyridin-4-yl)oxy)phenyl)-N-(1- (oxetan-3-yl)piperidin-4-yl)propanamide | 1E-a/1-(oxetan- 3-yl)- piperidin-4- amine | ¹H NMR (400 MHz, DMSO) δ 11.39 (s, 1H), 7.98 (d, J = 5.4 Hz, 1H), 7.70 (d, J = 7.3 Hz, 1H), 7.28-7.20 (m, 2H), 7.14- 7.06 (m, 2H), 6.16 (d, J = 5.5 Hz, 1H), 4.50 (t, J = 6.5 Hz, 2H), 4.41-4.36 (m, 2H), 3.56-3.47 (m, 1H), 3.45-3.39 (m, 3H), 2.87 (dd, J = 6.0, 13.3 Hz, 1H), 2.75- 2.66 (m, 2H), 2.59-2.55 (m, 2H), 2.39 (s, 3H), 1.91-1.78 (m, 2H), 1.70-1.59 (m, 2H), 1.43-1.28 (m, 2H). | Rt = 3.09 min, m/z 468.3 [M + H]⁺ (Method 1) |
| 4 | 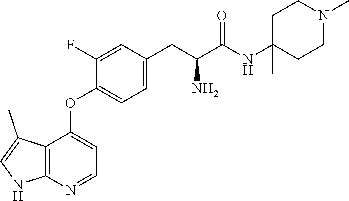 [SMILES: Cc1c[nH]c2nccc(Oc3ccc(C[C@H](N)C(=O)NC4(C)CCN(C)CC4)cc3F)c12] (S)-2-Amino-N-(1,4-dimethylpiperidin-4-yl)-3- (3-fluoro-4-((3-methyl-1H-pyrrolo[2,3-b]pyridin- 4-yl)oxy)phenyl)propanamide | 1E-a/1,4- dimethylpiperidin- 4-amine | ¹H NMR (400 MHz, DMSO) δ 11.41 (s, 1H), 8.21 (s, 1H), 7.99-7.97 (m, 1H), 7.43 (s, 1H), 7.34-7.23 (m, 2H), 7.15- 7.11 (m, 2H), 6.19-6.17 (m, 1H), 3.56 (t J = 6.95 Hz, 2H), 2.97-2.90 (m, 1H), 2.81-2.74 (m, 1H), 2.38-2.36 (m, 4H), 2.16-2.14 (m, 6H), 1.52-1.42 (m, 2H), 1.24-1.22 (m, 3H). | Rt = 2.13 min, m/z 440.3 [M + H]⁺ (Method 6) |
| 5 | 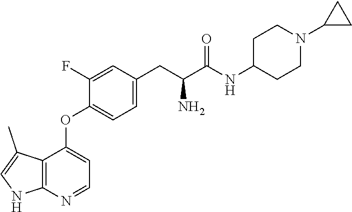 [SMILES: Cc1c[nH]c2nccc(Oc3ccc(C[C@H](N)C(=O)NC4CCN(C5CC5)CC4)cc3F)c12] (S)-2-Amino-N-(1-cyclopropylpiperidin-4-yl)-3- (3-fluoro-4-((3-methyl-1H-pyrrolo[2,3-b]pyridin- 4-yl)oxy)phenyl)propanamide | 1E-a/1-cyclo- propylpiperidin- 4-amine | ¹H NMR (400 MHz, DMSO) δ 11.38 (s, 1H), 7.97 (d, J = 5.44 Hz, 1H), 7.88 (d, J = 7.67 Hz, 1H), 7.28-7.20 (m, 2H), 7.14 (m, 1H), 7.08 (dd, J = 1.31, 8.27 Hz, 1H), 6.15 (d, J = 5.45 Hz, 1H), 3.56-3.47 (m, 1H), 3.38 (t, J = 6.78 Hz, 1H), 2.87 (q, J = 5.69, 13.25 Hz, 2H), 2.72-2.64 (m, 2H), 2.38 (s) and 2.37 (s) (together 3H), 2.24-2.16 (m, 2H), 1.79-1.76 (m, 2H), 1.67-1.53 (m, 3H), 1.35-1.16 (m, 2H), 0.42-0.36 (m, 2H), 0.28-0.22 (m, 2H). | Rt = 2.16 min, m/z 452.2 [M + H]⁺ (Method 1) |
| 6 | 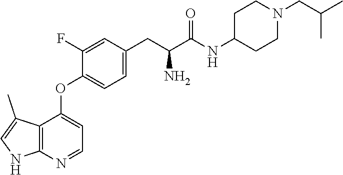 [SMILES: Cc1c[nH]c2nccc(Oc3ccc(C[C@H](N)C(=O)NC4CCN(CC(C)C)CC4)cc3F)c12] (S)-2-Amino-3-(3-fluoro-4-((3-methyl-1H- pyrrolo[2,3-b]pyridin-4-yl)oxy)phenyl)-N-(1- isobutylpiperidin-4-yl)propanamide | 1E-a/1- isbutylpiperidin- 4-amine | ¹H NMR (400 MHz, DMSO) δ 11.40 (s, 1H), 8.21 (s, 2H), 7.97 (d, J = 5.45 Hz, 1H), 7.78 (d, J = 7.74 Hz, 1H), 7.29-7.22 (m, 2H), 7.15-7-12 (m, 1H), 7.08 (d, J = 8.32 Hz, 1H), 6.16 (d, J = 5.47 Hz, 1H), 2.93-2.64 (m, 5H), 2.38 (s) and 2.37 (s) (together 3H), 2.02-1.89 (m, 4H), 1.76-1.57 (m, 3H), 1.43-1.23 (m, 2H), 0.83 (d, J = 6.52 Hz, 6H). | Rt = 2.19 min, m/z 468.5 [M + H]⁺ (Method 6) |
| 7 | 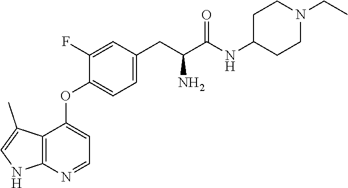 [SMILES: CCN1CCC(NC(=O)[C@@H](N)Cc2ccc(Oc3ccnc4[nH]cc(C)c34)c(F)c2)CC1] (S)-2-Amino-N-(1-ethylpiperidin-4-yl)-3-(3- fluoro-4-((3-methyl-1H-pyrrolo[2,3-b]pyridin-4- yl)oxy)phenyl)propanamide | 1E-a/1-ethyl- piperidin-4- amine | ¹H NMR (400 MHz, DMSO) δ 11.40 (s, 1H), 7.97 (d, J = 5.4 Hz, 1H), 7.65 (d, J = 7.9 Hz, 1H), 7.28-7.20 (m, 2H), 7.14 (d, J = 0.9 Hz, 1H), 7.08 (dd, J = 1.3, 8.3 Hz, 1H), 6.16-6.14 (m, 1H), 3.38 (t, J = 6.9 Hz, 1H), 2.87 (dd, J = 6.0, 13.2 Hz, 1H), 2.76-2.67 (m, 3H), 2.38 (d, J = 1.0 Hz, 3H), 2.27 (q, J = 7.2 Hz, 2H), 1.94- 1.87 (m, 2H), 1.76 (s, 2H), 1.67-1.56 (m, 2H), 1.41-1.24 (m, 2H), 0.96 (dd, J = 7.2, 7.2 Hz, 3H). | Rt = 2.46 min, m/z 440.3 [M + H]⁺ (Method 4) |
| 8 | 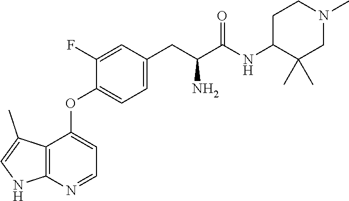 [SMILES: Cc1c[nH]c2nccc(Oc3ccc(C[C@H](N)C(=O)NC4CCN(C)CC4(C)C)cc3F)c12] (2S)-2-Amino-3-(3-fluoro-4-((3-methyl-1H- pyrrolo[2,3-b]pyridin-4-yl)oxy)phenyl)-N-(1,3,3- trimethylpiperidin-4-yl)propanamide | 1E-a/1,3,3- trimethyl- piperidin-4- amine | ¹H NMR (400 MHz, DMSO) δ 11.39 (s, 1H), 8.24 (br s, 0.3H, formate signal), 7.97 (d, J = 5.4 Hz, 1H), 7.48 (d, J = 9.6 Hz, 1H), 7.31-7.20 (m, 2H), 7.14-7.09 (m, 2H), 6.15 (d, J = 5.4 Hz, 1H), 3.52- 3.42 (m, 2H), 2.92 (dd, J = 5.7, 13.4 Hz, 2H), 2.75-2.63 (m, 2H), 2.38 (s, 3H), 2.36-2.30 (m, 2H), 2.11-2.09 (m, 3H), 1.91-1.82 (m, 1H), 1.71 (d, J = 11.1 Hz, 1H), 1.52-1.34 (m, 2H), 0.88 (s, 3H), 0.73 (s, 3H). | Rt = 1.77 min, m/z 454.3 [M + H]⁺ (Method 1) |
| 9 | 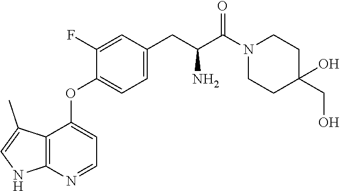 [SMILES: Cc1c[nH]c2nccc(Oc3ccc(C[C@H](N)C(=O)N4CCC(O)(CO)CC4)cc3F)c12] (S)-2-Amino-3-(3-fluoro-4-((3-methyl-1H- pyrrolo[2,3-b]pyridin-4-yl)oxy)phenyl)-1-(4- hydroxy-4-(hydroxymethyl)piperidin-1- yl)propan-1-one | 1E-a/4- (hydroxymethyl) piperidin-4-ol | ¹H NMR (400 MHz, DMSO) δ 11.40 (s, 1H), 7.98 (d J = 5.48 Hz, 1H), 7.34-7.27 (m, 1H), 7.26-7.18 (m, 1H), 7.15-7.07 (m, 2H), 6.15 (d J = 5.47 Hz, 1H), 4.63- 4.52 (m, 1H), 4.25 (s, 1H), 4.15 (d J = 12.32 Hz, 1H), 3.98-3.89 (m, 1H), 3.69 (d J = 12.28, 1H), 3.17 (d J = 5.58 Hz) and 3.13 (d J = 5.61 Hz) (together 2H), 3.09-2.98 (m, 1H), 2.92-2.73 (m, 2H), 2.71-2.59 (m, 1H), 2.38 (s, 3H), 1.62 (s, 2H), 1.58-0.98 (m, 2H). | Rt = 1.99 min, m/z 443.3 [M + H]⁺ (Method 1) |
| 10 | 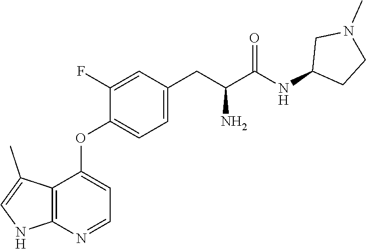 [SMILES: Cc1c[nH]c2nccc(Oc3ccc(C[C@H](N)C(=O)N[C@@H]4CCN(C)C4)cc3F)c12] (S)-2-Amino-3-(3-fluoro-4-((3-methyl-1H- pyrrolo[2,3-b]pyridin-4-yl)oxy)phenyl)-N-((R)-1- methylpyrrolidin-3-yl)propanamide | 1E-a/(R)-1-methyl- pyrrolidin-3-amine | ¹H NMR (400 MHz, DMSO) δ 11.39 (s, 1H), 7.97 (d, J = 5.43 Hz, 1H), 7.63 (d, J = 7.82 Hz, 1H), 7.29-7.20 (m, 2H), 7.14 (d, J = 0.87 Hz, 1H), 7.08 (d, J = 8.33 Hz, 1H), 6.17-6.15 (m, 1H), 4.19-4.10 (m, 1H), 3.41-3.35 (m, 1H), 2.90-2.63 (m, 2H), 2.58-2.52 (m, 2H), 2.39 (s) and 2.38 (s) (together 3H), 2.29-2.22 (m, 2H), 2.21 (s, 3H), 2.10-2.00 (m, 1H), 1.77 (s, 2H), 1.45-1.36 (m, 1H). | Rt = 2.08 min, m/z 412.3 [M + H]⁺ (Method 6) |
| 11 | 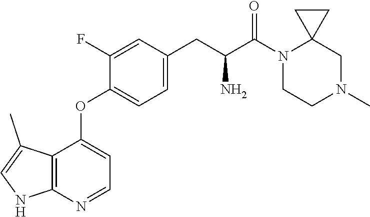 [SMILES: Cc1c[nH]c2nccc(Oc3ccc(C[C@H](N)C(=O)N4CCN(C)CC45CC5)cc3F)c12] (S)-2-Amino-3-(3-fluoro-4-((3-methyl-1H- pyrrolo[2,3-b]pyridin-4-yl)oxy)phenyl)-1-(7- methyl-4,7-diazaspiro[2.5]octan-4-yl)propan-1- one | 1E-a/7-methyl-4,7- diazaspiro[2.5] octane | ¹H NMR (400 MHz, DMSO) δ 11.42 (s, 1H), 7.99 (d, J = 5.5 Hz, 1H), 7.32-7.24 (m, 2H), 7.16-7.10 (m, 2H), 6.18 (d, J = 4.8 Hz, 1H), 4.06-4.05 (m, 1H), 2.97 (s, 1H), 2.83-2.67 (m, 2H), 2.40-2.35 (m, 4H), 2.24-2.22 (m, 2H), 2.11-2.08 (m, 2H), 1.99-1.90 (m, 3H), 1.63 (s, 1H), 1.33-1.31 (m, 1H), 0.98 (s, 1H), 0.51-0.50 (m, 3H). | Rt = 1.86 min, m/z 438.0 [M + H]⁺ (Method 1) |
| 12 | 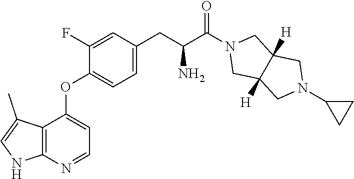 [SMILES: Cc1c[nH]c2nccc(Oc3ccc(C[C@H](N)C(=O)N4C[C@@H]5CN(C6CC6)C[C@@H]5C4)cc3F)c12] (S)-2-Amino-1-((3aR,6aS)-5- cyclopropylhexahydropyrrolo[3,4-c]pyrrol-2(1H)- yl)-3-(3-fluoro-4-((3-methyl-1H-pyrrolo[2,3- b]pyridin-4-yl)oxy)phenyl)propan-1-one | 1E-a/(3aR,6aS)-2- cyclopropylocta- hydropyrrolo- [3,4-c]pyrrole | ¹H NMR (400 MHz, DMSO) δ 11.39 (s, 1H), 7.98 (d, J = 5.5 Hz, 1H), 7.33-7.20 (m, 2H), 7.15-7.08 (m, 2H), 6.13 (d, J = 5.4 Hz, 1H), 3.74-3.63 (m, 1H), 3.56- 3.35 (m, 2H), 3.20-3.00 (m, 2H), 2.84- 2.73 (m, 2H), 2.69-2.61 (m, 4H), 2.38 (d, J = 1.9 Hz, 3H), 2.36-2.27 (m, 1H), 1.72-1.72 (m, 2H), 1.61-1.50 (m, 1H), 0.39-0.22 (m, 4H). | Rt = 1.84 min, m/z 464.3 [M + H]⁺ (Method 1) |
| 13 | 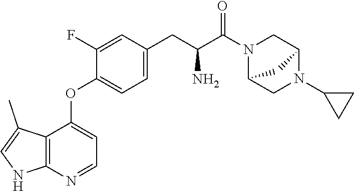 [SMILES: Cc1c[nH]c2nccc(Oc3ccc(C[C@H](N)C(=O)N4C[C@@H]5C[C@H]4CN5C4CC4)cc3F)c12] (S)-2-Amino-1-((1S,4S)-5-cyclopropyl-2,5- diazabicyclo[2.2.1]heptan-2-yl)-3-(3-fluoro-4-((3- methyl-1H-pyrrolo[2,3-b]pyridin-4- yl)oxy)phenyl)propan-1-one | 1E-a/(1S,4S)-2- cyclopropyl- 2,5-diazabicyclo [2.2.1]heptane | ¹H NMR (400 MHz, DMSO) δ 11.39 (s, 1H), 7.98 (dd, J = 5.4, 12.8 Hz, 1H), 7.36- 7.20 (m, 2H), 7.15-7.10 (m, 2H), 6.11 (m, 1H), 4.53-4.27 (m, 1H), 3.75-3.57 (m, 1H), 3.46-3.34 (m, 2H), 3.21-3.03 (m, 1H), 2.89-2.66 (m, 3H), 2.38 (s, 3H), 2.23-1.89 (m, 1H), 1.72-1.30 (m, 3H), 0.39-0.19 (m, 4H). | Rt = 1.81 min, m/z 450.2 [M + H]⁺ (Method 1) |
| 14 | 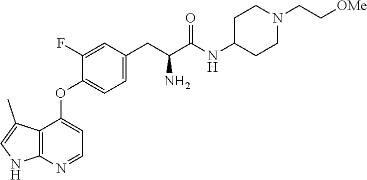 [SMILES: COCCN1CCC(NC(=O)[C@@H](N)Cc2ccc(Oc3ccnc4[nH]cc(C)c34)c(F)c2)CC1] (S)-2-Amino-3-(3-fluoro-4-((3-methyl-1H- pyrrolo[2,3-b]pyridin-4-yl)oxy)phenyl)-N-(1-(2- methoxyethyl)piperidin-4-yl)propanamide ee % (n.d.) | 1E-a/1-(2- methoxyethyl)- piperidin-4-amine | ¹H NMR (400 MHz, DMSO) δ 11.40 (s, 1H), 7.99 (d J = 5.4 Hz, 1H), 7.66 (d J = 7.9 Hz, 1H), 7.30-7.22 (m, 2H), 7.17- 7.14 (m, 1H), 7.12-7.08 (m, 1H), 6.17 (dd J = 5.4, 0.9 Hz, 1H), 3.56-3.46 (m, 1H), 3.41 (t J = 5.9 Hz, 2H), 3.24 (s, 3H), 2.91-2.84 (m, 1H), 2.80-2.68 (m, 2H), 2.44 (t J = 5.9 Hz, 2H), 2.40 (d J = 1.1 Hz, 3H), 2.08-1.97 (m, 2H), 1.84-1.70 (m, 2H), 1.69-1.55 (m, 2H) 1.42-1.24 (m, 2H). | Rt = 1.84 min, m/z 470.5 [M + H]⁺ (Method 8) |
| 15 | 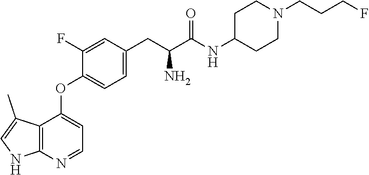 [SMILES: Cc1c[nH]c2nccc(Oc3ccc(C[C@H](N)C(=O)NC4CCN(CCCF)CC4)cc3F)c12] (S)-2-Amino-3-(3-fluoro-4-((3-methyl-1H- pyrrolo[2,3-b]pyridin-4-yl)oxy)phenyl)-N-(1-(3- fluoropropyl)piperidin-4-yl)propanamide | 1E-a/1-(3- fluoropropyl))- piperidin-4-yl amine | ¹H NMR (400 MHz, DMSO) δ 11.38 (s, 1H), 7.96 (d J = 5.4 Hz, 1H), 7.64 (d J = 7.7 Hz, 1H), 7.28-7.18 (m, 2H), 7.12 (s, 1H), 7.07 (d J = 8.6 Hz, 1H), 6.14 (d J = 5.4 Hz, 1H), 4.44 (dt J = 50.8, 6.0 Hz, 2H), 3.54-3.44 (m, 1H), 3.36 (t J = 6.5 Hz, 1H), 2.88-2.82 (m, 1H), 2.76-2.64 (m, 3H), 2.37 (s, 3H), 2.32 (t J = 7.2 Hz, 2H), 1.98-1.88 (m, 2H), 1.84-1.54 (m, 6H) 1.41-1.22 (m, 2H). | Rt = 1.84 min, m/z 472.4 [M + H]⁺ (Method 8) |
| 16 | 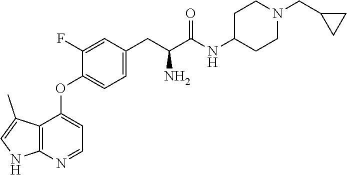 [SMILES: Cc1c[nH]c2nccc(Oc3ccc(C[C@H](N)C(=O)NC4CCN(CC5CC5)CC4)cc3F)c12] (S)-2-Amino-N-(1-(cyclopropylmethyl)piperidin- 4-yl)-3-(3-fluoro-4-((3-methyl-1H-pyrrolo[2,3- b]pyridin-4-yl)oxy)phenyl)propanamide | 1E-a/1- (cyclopropylmethyl)- piperidin-4-amine | ¹H NMR (400 MHz, DMSO) δ 11.36- 11.35 (m, 1H), 7.93 (d, J = 5.4 Hz, 1H), 7.61 (d, J = 7.9 Hz, 1H), 7.24-7.16 (m, 2H), 7.10-7.02 (m, 2H), 6.11 (dd, J = 0.8, 5.4 Hz, 1H), 3.34 (t, J = 6.7 Hz, 1H), 2.85-2.62 (m, 4H), 2.34 (d, J = 1.1 Hz, 3H), 2.10-2.07 (m, 2H), 1.96-1.89 (m, 2H), 1.77-1.73 (m, 2H), 1.63-1.53 (m, 2H), 1.37-1.22 (m, 2H), 0.79-0.70 (m, 1H), 0.42-0.36 (m, 2H), 0.02-0.03 (2H, m). | Rt = 1.95 min, m/z 466.3 [M + H]⁺ (Method 1) |
| 17 | 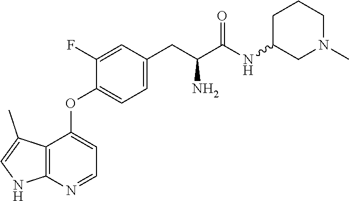 [SMILES: Cc1c[nH]c2nccc(Oc3ccc(C[C@H](N)C(=O)NC4CCCN(C)C4)cc3F)c12] (2S)-2-Amino-3-(3-fluoro-4-((3-methyl-1H- pyrrolo[2,3-b]pyridin-4-yl)oxy)phenyl)-N-(1- methylpiperidin-3-yl)propanamide (diastereomer 1) | 1E-a/rac 1- methylpiperidin-3- amine | ¹H NMR (400 MHz, DMSO) δ 11.40 (s, 1H), 8.00 (d, J = 5.5 Hz, 1H), 7.79-7.74 (m, 1H), 7.30-7.23 (m, 2H), 7.17-7.08 (m, 2H), 6.18 (d, J = 5.5 Hz, 1H), 3.76- 3.69 (m, 1H), 3.47 (t, J = 6.7 Hz, 1H), 2.93-2.86 (m, 1H), 2.78 (dd, J = 7.2, 13.3 Hz, 1H), 2.40-2.39 (m, 6H), 2.11- 2.10 (m, 3H), 2.04-1.94 (m, 1H), 1.81- 1.69 (m, 1H), 1.59-1.56 (m, 2H), 1.46- 1.38 (m, 1H), 1.26-1.16 (m, 1H). | Rt = 1.70 min, m/z 426.4 [M + H]⁺ (Method 8) |
| 18 | 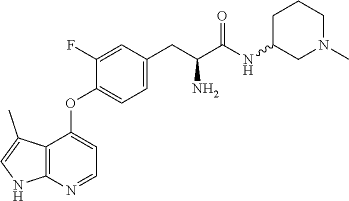 [SMILES: Cc1c[nH]c2nccc(Oc3ccc(C[C@H](N)C(=O)NC4CCCN(C)C4)cc3F)c12] (2S)-2-Amino-3-(3-fluoro-4-((3-methyl-1H- pyrrolo[2,3-b]pyridin-4-yl)oxy)phenyl)-N-(1- methylpiperidin-3-yl)propanamide (diastereomer 2) | 1E-a/rac 1- methylpiperidin-3- amine | ¹H NMR (400 MHz, DMSO) δ 11.41 (s, 1H), 7.99 (d, J = 5.4 Hz, 1H), 7.83-7.78 (m, 1H), 7.32-7.23 (m, 2H), 7.17-7.09 (m, 2H), 6.19 (d, J = 5.5 Hz, 1H), 3.78- 3.71 (m, 1H), 3.53 (t, J = 6.7 Hz, 1H), 2.93 (dd, J = 5.8, 13.3 Hz, 1H), 2.78 (dd, J = 7.5, 13.3 Hz, 1H), 2.48-2.30 (m, 6H), 2.14 (s, 3H), 2.05-2.01 (m, 1H), 1.89-1.86 (m, 1H), 1.60-1.39 (m, 3H), 1.20-1.12 (m, 1H). | Rt = 1.85 min, m/z 426.4 [M + H]⁺ (Method 8) |
| 19 | 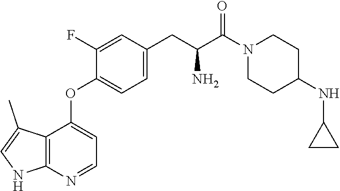 [SMILES: Cc1c[nH]c2nccc(Oc3ccc(C[C@H](N)C(=O)N4CCC(NC5CC5)CC4)cc3F)c12] (S)-2-Amino-1-(4-(cyclopropylamino)piperidin- 1-yl)-3-(3-fluoro-4-((3-methyl-1H-pyrrolo[2,3- b]pyridin-4-yl)oxy)phenyl)propan-1-one | 1E-a/Intermediate 19C | ¹H NMR (400 MHz, DMSO) δ 11.39 (d, J = 4.4 Hz, 1H), 7.99 (d, J = 5.4 Hz, 1H), 7.33-7.28 (m, 1H), 7.23 (dd, J = 8.4, 8.4 Hz, 1H), 7.14-7.08 (m, 2H), 6.16 (dd, J = 5.5, 11.0 Hz, 1H), 4.14 (dd, J = 13.0, 23.3 Hz, 1H), 3.95 (q, J = 7.3 Hz, 1H), 3.79 (d, J = 12.7 Hz, 1H), 3.08-2.89 (m, 1H), 2.83-2.60 (m, 4H), 2.38 (s, 3H), 2.08-2.01 (m, 1H), 1.81-1.69 (m, 4H), 1.28-1.08 (m, 1H), 1.02-0.75 (m, 1H), 0.38-0.32 (m, 2H), 0.20-0.14 (m, 2H). | Rt = 1.85 min, m/z 452.4 [M + H]⁺ (Method 1) |
| 20 | 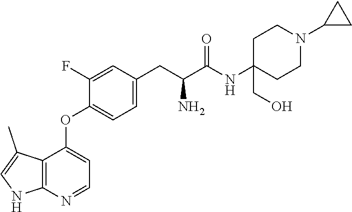 [SMILES: Cc1c[nH]c2nccc(Oc3ccc(C[C@H](N)C(=O)NC4(CO)CCN(C5CC5)CC4)cc3F)c12] (S)-2-Amino-N-(1-cyclopropyl-4- (hydroxymethyl)piperidin-4-yl)-3-(3-fluoro-4-((3- methyl-1H-pyrrolo [2,3-b]pyridin-4- yl)oxy)phenyl)propanamide | 1E-a/Intermediate 20D | ¹H NMR (400 MHz, DMSO) δ 11.15 (s, 1H), 7.74 (d, J = 5.4 Hz, 1H), 7.13-6.99 (m, 3H), 6.93-6.89 (m, 2H), 5.93 (d, J = 3.3 Hz, 1H), 4.46 (s, 1H), 3.31-3.21 (m, 5H), 2.75-2.69 (m, 1H), 2.52-2.43 (m, 2H), 2.40-2.33 (m, 2H), 2.14-2.12 (m, 3H), 2.01-1.85 (m, 2H), 1.79-1.67 (m, 2H), 1.30-1.13 (m, 2H), 0.13-0.09 (m, 2H), 0.01-0.03 (m, 2H). | Rt = 1.80 min, m/z 482.2 [M + H]⁺ (Method 1) |
| 21 | 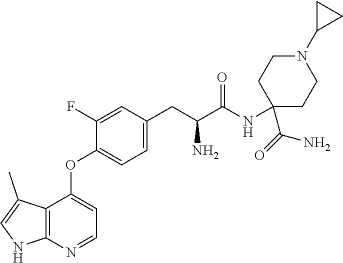 [SMILES: Cc1c[nH]c2nccc(Oc3ccc(C[C@H](N)C(=O)NC4(C(N)=O)CCN(C5CC5)CC4)cc3F)c12] (S)-4-(2-Amino-3-(3-fluoro-4-((3-methyl-1H- pyrrolo[2,3-b]pyridin-4- yl)oxy)phenyl)propanamido)-1- cyclopropylpiperidine-4-carboxamide | 1E-a/Intermediate 21D | ¹H NMR (400 MHz, DMSO) δ 11.2 (s, 1H), 7.74 (d, J = 5.2 Hz, 1H), 7.50 (s, 1H), 7.11 (dd, J = 11.2, 2.0 Hz, 1H), 7.02 (t, J = 8.3 Hz, 1H), 6.93-6.88 (m, 2H), 6.74 (s, 1H), 6.61 (s, 1H), 5.94 (dd, J = 5.4, 1.0 Hz, 1H), 3.35-3.28 (m, 1H), 2.73 (d, 13.1, 5.6 Hz, 1H), 2.52-2.37 (m, 3H), 2.13 (d, J = 1.1 Hz, 3H), 2.00-1.91 (m, 1H), 1.85-1.46 (m, 7H), 1.30-1.23 (m, 1H), 0.14-0.08 (m, 2H), 0.03 (m, 2H) | Rt = 3.22 min, m/z 495.4 [M + H]⁺ (Method 5) |
| 22 | 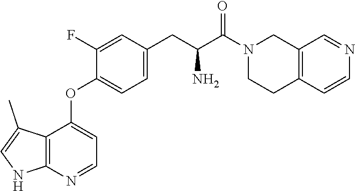 [SMILES: Cc1c[nH]c2nccc(Oc3ccc(C[C@H](N)C(=O)N4CCc5ccncc5C4)cc3F)c12] (S)-2-Amino-1-(3,4-dihydro-2,7-naphthyridin- 2(1H)-yl)-3-(3-fluoro-4-((3-methyl-1H- pyrrolo[2,3-b]pyridin-4-yl)oxy)phenyl)propan-1- one | 1E-a/1,2,3,4- tetrahydro-2,7- naphthyridine | ¹H NMR (400 MHz, DMSO) δ 11.39 (s, 1H), 8.42 (s) and 8.30 (s) (together 1H), 7.93 (t, J = 5.67 Hz, 1H), 7.33 (d, J = 11.87 Hz and 7.28 (d J = 11.87 Hz,) (together 1H), 7.22-7.05 (m, 4H), 6.08 (d, J = 5.43 Hz) and 6.02 (d, J = 5.33 Hz) (together 1H), 4.84-4.51 (m, 2H), 4.15- 3.95 (m, 1H), 3.92-3.48 (m, 2H), 2.97- 2.58 (m, 4H), 2.38 (s) and 2.37 (s) (together 3H). 1.80 (s, 2H) (1 H not seen/obscured by solvent.) | Rt = 1.86 min, m/z 446.3 [M + H]⁺ (Method 1) |
| 23 | 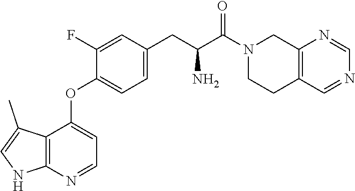 [SMILES: Cc1c[nH]c2nccc(Oc3ccc(C[C@H](N)C(=O)N4CCc5cncnc5C4)cc3F)c12] (S)-2-Amino-1-(5,8-dihydropyrido[3,4- d]pyrimidin-7(6H)-yl)-3-(3-fluoro-4-((3-methyl- 1H-pyrrolo[2,3-b]pyridin-4- yl)oxy)phenyl)propan-1-one | 1E-a/5,6,7,8- tetrahydropyrido [3,4-d]- pyrimidine | ¹H NMR (400 MHz, DMSO) δ 11.38 (s) and 11.37(s) (together 1H), 8.96 (d, J = 5.88 Hz, 1H), 8.59 (d, J = 10.37 Hz, 1H 7.97-7.90 (m, 1H), 7.40-6.99 (m, 4H), 6.09 (d, J = 5.37 Hz) and 6.00 (d, J = 5.37 Hz) (together 1H), 4.77-4.42 (m, 2H), 4.13-4.0 (m, 1H), 3.99-3.45 (m, 2H), 2.97-2.56 (m, 4H), 2.39 (s) and 2.37 (s) (together 3H), 1.81 (s, 2H) | Rt = 3.02 min, m/z 447.4 [M + H]⁺ (Method 5) |
| 24 | 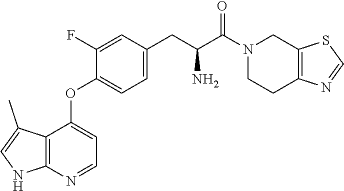 [SMILES: Cc1c[nH]c2nccc(Oc3ccc(C[C@H](N)C(=O)N4CCc5ncsc5C4)cc3F)c12] (S)-2-Amino-1-(6,7-dihydrothiazolo[5,4- c]pyridin-5(4H)-yl)-3-(3-fluoro-4-((3-methyl-1H- pyrrolo[2,3-b]pyridin-4-yl)oxy)phenyl)propan-1- one | 1E-a/4,5,6,7- tetrahydrothiazolo [5,4-c]- pyridine | ¹H NMR (400 MHz, DMSO) δ 11.38 (s, 1H), 8.96 (s), 8.95 (s) (together 1H), 7.96 (t, J = 5.61 Hz, 1H), 7.38-7.02(m, 4H), 6.11 (d, J = 5.36 Hz) and 6.06 (d, J = 5.38 Hz) (together 1H), 4.89-4.63 (m, 2H), 4.09-3.98 (m, 1H), 3.94-3.66 (m, 2H), 2.97-2.62 (m, 4H), 2.38 (s) and 2.37 (s) (together 3H), 2.07 (s, 2H) | Rt = 2.82 min, m/z 452.0 [M + H]⁺ (Method 5) |
| 25 | 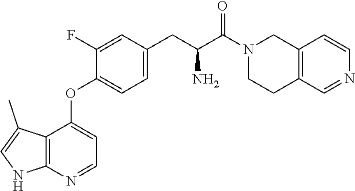 [SMILES: Cc1c[nH]c2nccc(Oc3ccc(C[C@H](N)C(=O)N4CCc5cnccc5C4)cc3F)c12] (S)-2-Amino-1-(3,4-dihydro-2,6-naphthyridin- 2(1H)-yl)-3-(3-fluoro-4-((3-methyl-1H- pyrrolo[2,3-b]pyridin-4-yl)oxy)phenyl)propan-1- one | 1E-a/1,2,3,4- tetrahydro-2,6- naphthyridine | ¹H NMR (400 MHz, DMSO) δ 11.40 (s, 1H), 8.40-8.32 (m, 2H), 7.94 (t J = 6.3 Hz, 1H), 7.38-7.06 (m, 5H), 6.09 (d J = 5.3 Hz and 6.02 (d J = 5.5 Hz) (together 1H), 4.80-4.56 (m, 2H) 4.06 (t J = 6.9 Hz, 1H), 3.90-3.56 (m, 2H), 2.94-2.60 (m, 4H), 2.39 (s, 3H), 1.86 (br s, 2H) | Rt = 1.85 min, m/z 446.4 [M + H]⁺ (Method 8) |
| 26 | 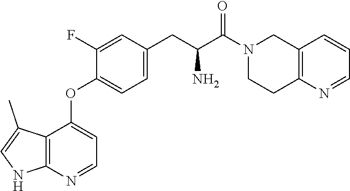 [SMILES: Cc1c[nH]c2nccc(Oc3ccc(C[C@H](N)C(=O)N4CCc5ncccc5C4)cc3F)c12] (S)-2-amino-1-(7,8-dihydro-1,6-naphthyridin- 6(5H)-yl)-3-(3-fluoro-4-((3-methyl-1H- pyrrolo[2,3-b]pyridin-4-yl)oxy)phenyl)propan-1- one | 1E-a/5,6,7,8- tetrahydro-1,6- naphthyridine | ¹H NMR (400 MHz, DMSO) δ 11.39 (s, 1H), 8.40-8.36 (m, 1H), 7.99-7.92 (m, 1H), 7.64 (d J = 7.5 Hz) and 7.54 (d J = 7.5 Hz) (together 1H), 7.39-7.06 (m, 5H), 6.16 (d J = 5.5 Hz) and 6.12 (d J = 5.2 Hz) (together 1H), 4.80-4.56 (m, 2H), 4.12- 4.04 (m1H), 3.96-3.64 (m, 2H) 3.04- 2.68 (m, 4H), 2.38 (s, 3H), 2.20-1.80 (brs, 2H) | Rt = 1.95 min, m/z 446.2 [M + H]⁺ (Method 8) |
| 27 | 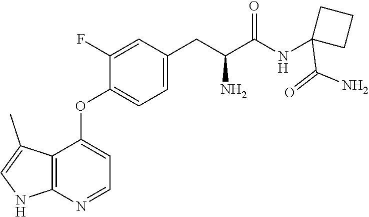 [SMILES: Cc1c[nH]c2nccc(Oc3ccc(C[C@H](N)C(=O)NC4(C(N)=O)CCC4)cc3F)c12] (S)-1-(2-Amino-3-(3-fluoro-4-((3-methyl-1H- pyrrolo[2,3-b]pyridin-4- yl)oxy)phenyl)propanamido)cyclobutane-1- carboxamide | 1E-a/1-aminocyclo- butane-1- carboxamide | ¹H NMR (400 MHz, DMSO) δ 11.40 (s, 1H), 8.38 (s, 1H), 8.20 (s, 1H), 7.98 (d J = 5.46 Hz, 1H), 7.34-7.23 (m, 2H), 7.14-7.11 (m, 2H), 6.93-6.85 (m, 2H), 6.18 (d J = 5.45 Hz, 1H), 3.07-2.93 (m, 2H), 2.79 (dd J = 7.56, 13.40 Hz, 1H), 2.39-2.37 (m, 4H), 2.07-1.91 (m, 2H), 1.84-1.71 (m, 2H). | Rt = 2.27 min, m/z 426.3 [M + H]⁺ (Method 6) |
| 28 | 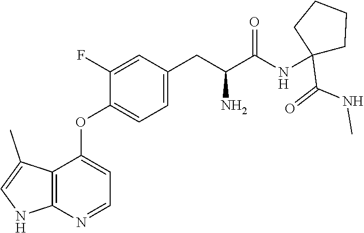 [SMILES: CNC(=O)C1(NC(=O)[C@@H](N)Cc2ccc(Oc3ccnc4[nH]cc(C)c34)c(F)c2)CCCC1] (S)-1-(2-amino-3-(3-fluoro-4-((3-methyl-1H- pyrrolo[2,3-b]pyridin-4- yl)oxy)phenyl)propanamido)-N- methylcyclopentane-1-carboxamide | 1E-a/1-amino-N- methylcyclopentane- 1-carboxamide | ¹H NMR (400 MHz, DMSO) δ 11.40 (s, 1H), 8.22 (s, 1H), 7.99-7.94 (m, 2H), 7.39 (q, J = 4.45 Hz, 1H), 7.31-7.22 (m, 2H), 7.15-7.08 (m, 2H), 6.18-6.16 (m, 1H), 3.54 (t J = 6.95 Hz, 2H), 3.21-3.05 (m, 1H), 2.90 (dd J = 6.35, 13.38 Hz, 1H), 2.76 (dd J = 7.19, 13.46 Hz, 1H), 2.55 (d, J = 4.53 Hz, 2H), 2.38 (d J = 1.02 Hz, 3H), 2.08-2.00 (m, 1H), 1.95-1.74 (m, 3H), 1.61-1.39 (m, 4H). | Rt = 0.85 min, m/z 454.3 [M + H]⁺ (Method 6) |
| 29 | 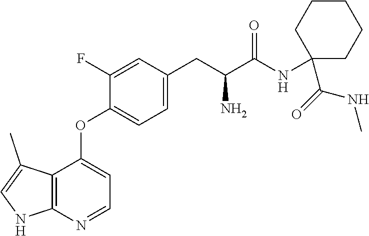 [SMILES: CNC(=O)C1(NC(=O)[C@@H](N)Cc2ccc(Oc3ccnc4[nH]cc(C)c34)c(F)c2)CCCCC1] Exact Mass: 467.23 (S)-1-(2-amino-3-(3-fluoro-4-((3-methyl-1H- pyrrolo[2,3-b]pyridin-4- yl)oxy)phenyl)propanamido)-N- methylcyclohexane-1-carboxamide | 1E-a/1-amino-N- methylcyclo- hexane-1- carboxamide | ¹H NMR (400 MHz, DMSO) δ 11.40 (s, 1H), 7.98 (d, J = 5.45 Hz, 1H), 7.68 (s, 1H), 7.36-7.30 (m, 2H), 7.25 (t, J = 8.38 Hz, 1H), 7.15-7.14 (m, 2H), 6.17 (d, J = 5.50 Hz, 1H), 3.58-3.52 (m, 1H), 3.17 (d, J = 5.12 Hz, 1H), 2.95 (dd, J = 5.66, 13.52 Hz, 1H), 2.75-2.67 (m, 1H), 2.54 (d, J = 4.59 Hz, 3H), 2.38 (s) and 2.37 (s) (together 3H), 2.07-1.83 (m, m, 3H), 1.66-1.55 (m, 2H), 1.46-1.44 (m, 3H), 1.30 (d, J = 12.30 Hz, 1H), 1.18-1.11 (m, 2H). | Rt = 2.46 min, m/z 468.2 [M + H]⁺ (Method 1) |
| 30 | 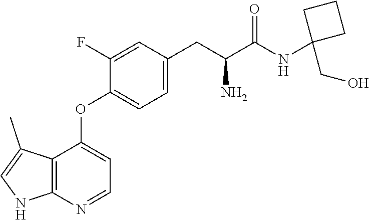 [SMILES: Cc1c[nH]c2nccc(Oc3ccc(C[C@H](N)C(=O)NC4(CO)CCC4)cc3F)c12] (S)-2-amino-3-(3-fluoro-4-((3 -methyl-1H- pyrrolo[2,3-b]pyridin-4-yl)oxy)phenyl)-N-(1- (hydroxymethyl)cyclobutyl)propanamide | 1E-a/(1- aminocyclobutyl)- methanol | ¹H NMR (400 MHz, DMSO) δ 11.39 (s, 1H), 8.22 (s, 1H), 7.98 (d J = 5.45 Hz, 1H), 7.87 (s, 1H), 7.33-7.22 (m, 2H), 7.15-7.10 (m, 2H), 6.18 (d J = 5.46 Hz, 1H), 3.52-3.44 (m, 5H), 2.94 (dd, J = 5.52, 13.42 Hz, 1H), 2.77-2.66 (m, 1H), 2.39 (s) and 2.37 (s) (together 3H), 2.19-2.08 (m, 2H), 2.04-1.95 (m, 2H), 1.80-1.60 (m, 2H). | Rt = 2.32 min, m/z 413.3 [M + H]⁺ (Method 6) |
| 31 | 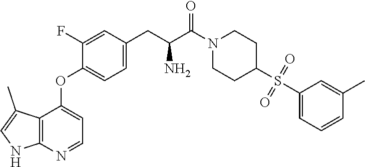 [SMILES: Cc1cccc(S(=O)(=O)C2CCN(C(=O)[C@@H](N)Cc3ccc(Oc4ccnc5[nH]cc(C)c45)c(F)c3)CC2)c1] (S)-2-Amino-3-(3-fluoro-4-((3-methyl-1H- pyrrolo[2,3-b]pyridin-4-yl)oxy)phenyl)-1-(4-(m- tolylsulfonyl)piperidin-1-yl)propan-1-one | 1E-a/Intermediate 31C | ¹H NMR (400 MHz, DMSO) δ (1032850) 11.38 (s, 1H), 7.98 (dd, J = 11.8, 5.7 Hz, 1H), 7.67-7.54 (m, 4H), 7.31 (d, J = 7.3 Hz, 1H), 7.24-7.17 (m, 1H), 7.15-7.11 (m, 1H), 7.11-7.04 (m, 1H), 6.20-6.10 (m, 1H), 4.52-4.42 (br m, 1H), 4.12-4.00 (m, 1H), 3.95-3.86 (m, 1H), 3.59-3.45 (m, 1H), 3.05-2.75 (m, 2H), 2.70-2.53 (m, 1H), 2.45-2.34 (m, 6H),1.90-1.70 (m, 4H), 1.60-1.46 (m, 1H), 1.40-1.04 (m, 2H). | Rt = 3.05 min, m/z 551.3 [M + H]⁺ (Method 1) |
| 32 | 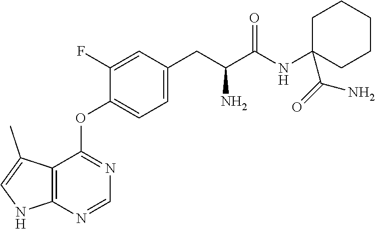 [SMILES: Cc1c[nH]c2ncnc(Oc3ccc(C[C@H](N)C(=O)NC4(C(N)=O)CCCCC4)cc3F)c12] (S)-1-(2-amino-3-(3-fluoro-4-((5-methyl-7H- pyrrolo[2,3-d]pyrimidin-4-yl)oxy)phenyl)- propanamido)cyclohexanecarboxamide | 1E-b/1-amino- cyclohexane- 1-carboxamide | ¹H NMR (400 MHz, DMSO) δ 11.91- 11.91 (m, 1H), 8.20 (s, 1H), 7.71 (s, 1H), 7.34-7.26 (m, 2H), 7.24 (s, 1H), 7.14 (dd, J = 1.3, 8.2 Hz, 1H), 6.93 (s, 1H), 6.80 (s, 1H), 3.53 (dd, J = 5.0, 8.5 Hz, 1H), 2.99 (dd, J = 4.9, 13.6 Hz, 1H), 2.70-2.63 (m, 1H), 2.42 (d, J = 1.0 Hz, 3H), 2.08-1.95 (m, 3H), 1.66-1.56 (m, 2H), 1.50-1.41 (m, 3H), 1.36-1.20 (m, 3H). | Rt = 2.90 min, m/z 477.1 [M + Na]⁺ (Method 1) |
| 33 | 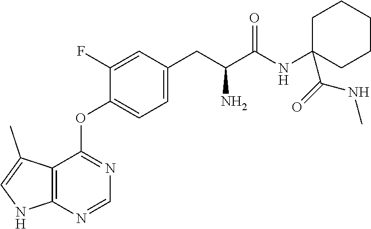 [SMILES: CNC(=O)C1(NC(=O)[C@@H](N)Cc2ccc(Oc3ncnc4[nH]cc(C)c34)c(F)c2)CCCCC1] (S)-1-(2-amino-3-(3-fluoro-4-((5-methyl-7H- pyrrolo[2,3-d]pyrimidin-4-yl)oxy)phenyl)- propanamido)-N-methylcyclohexanecarboxamide | 1E-b/1-amino-N- methylcyclohexane- 1-carboxamide | ¹H NMR (400 MHz, DMSO) δ 11.92 (s, 1H), 8.20 (s, 1H), 7.73 (s, 1H), 7.36- 7.26 (m, 3H), 7.23 (s, 1H), 7.15 (dd, J = 1.1, 8.2 Hz, 1H), 3.56 (dd, J = 5.2, 8.2 Hz, 1H), 2.99 (dd, J = 5.4, 13.5 Hz, 1H), 2.69 (dd, J = 8.4, 13.5 Hz, 1H), 2.55 (d, J = 4.6 Hz, 3H), 2.42-2.41 (m, 3H), 2.07- 1.89 (m, 3H), 1.66-1.13 (m, 9H). | Rt = 2.93 min, m/z 469.4 [M + H]⁺ (Method 1) |
| 34 | 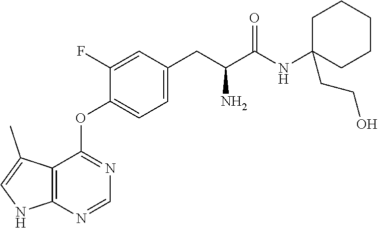 [SMILES: Cc1c[nH]c2ncnc(Oc3ccc(C[C@H](N)C(=O)NC4(CCO)CCCCC4)cc3F)c12] (S)-2-amino-3-(3-fluoro-4-((5-methyl-7H- pyrrolo[2,3-d]pyrimidin-4-yl)oxy)phenyl)-N-(1- (2-hydroxyethyl)cyclohexyl)propanamide | 1E-b/2-(1- aminocyclohexyl)- ethan-1-ol | ¹H NMR (400 MHz, DMSO) δ 11.91 (s, 1H), 8.20 (s, 1H), 7.34-7.26 (m, 3H), 7.24 (s, 1H), 7.13 (dd, J = 1.2, 8.2 Hz, 1H), 4.25 (s, 1H), 3.46-3.42 (m, 3H), 2.98 (dd, J = 4.7, 13.6 Hz, 1H), 2.62 (dd, J = 8.8, 13.6 Hz, 1H), 2.42 (s, 3H), 2.12- 2.03 (m, 3H), 1.90-1.79 (m, 2H), 1.48- 1.19 (m, 8H). | Rt = 3.13 min, m/z 478.0 [M + Na]⁺ (Method 1) |
| 35 | 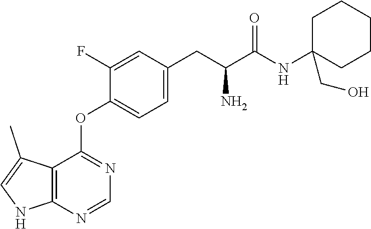 [SMILES: Cc1c[nH]c2ncnc(Oc3ccc(C[C@H](N)C(=O)NC4(CO)CCCCC4)cc3F)c12] (S)-2-Amino-3-(3-fluoro-4-((5-methyl-7H- pyrrolo[2,3-d]pyrimidin-4-yl)oxy)phenyl)-N-(1- (hydroxymethyl)cyclohexyl)propanamide | 1E-b/(1- aminocyclohexyl)- methanol | ¹H NMR (400 MHz, DMSO) δ 11.91 (s, 1H), 8.20 (s, 1H), 7.36-7.25 (m, 3H), 7.24 (s, 1H), 7.13 (dd, J = 1.4, 8.1 Hz, 1H), 4.73 (dd, J = 5.9, 5.9 Hz, 1H), 3.46- 3.42 (m, 3H), 2.98 (dd, J = 4.8, 13.5 Hz, 1H), 2.64 (dd, J = 8.4, 13.5 Hz, 1H), 2.42 (d, J = 1.0 Hz, 3H), 2.01-1.87 (m, 4H), 1.49-1.15 (m, 8H). | Rt = 2.95 min, m/z 442.0 [M + H]⁺ (Method 4) |
| 36 | 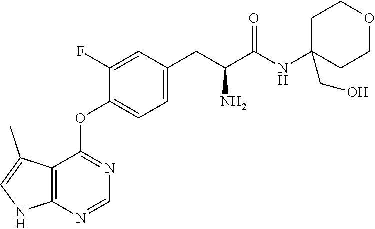 [SMILES: Cc1c[nH]c2ncnc(Oc3ccc(C[C@H](N)C(=O)NC4(CO)CCOCC4)cc3F)c12] (S)-2-Amino-3-(3-fluoro-4-((5-methyl-7H- pyrrolo[2,3-d]pyrimidin-4-yl)oxy)phenyl)-N-(4- (hydroxymethyl)tetrahydro-2H-pyran-4- yl)propanamide | 1E-b/(4- aminotetrahydro- 2H-pyran-4-yl) methanol | ¹H NMR (400 MHz, DMSO) δ 11.92 (s, 1H), 8.20 (s, 1H), 7.47 (s, 1H), 7.34- 7.23 (m, 3H), 7.13 (dd, J = 1.4, 8.3 Hz, 1H), 4.77 (dd, J = 5.9, 5.9 Hz, 1H), 3.64- 3.58 (m, 2H), 3.52-3.38 (m, 5H), 2.97 (dd, J = 4.9, 13.4 Hz, 1H), 2.69-2.62 (m, 1H), 2.42 (s, 3H), 1.98-1.93 (m, 2H), 1.84 (s, 2H), 1.60-1.50 (m, 2H). | Rt = 2.68 min, m/z 444.0 [M + H]⁺ (Method 4) |
| 37 | 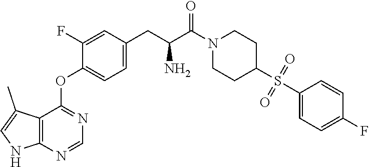 [SMILES: Cc1c[nH]c2ncnc(Oc3ccc(C[C@H](N)C(=O)N4CCC(S(=O)(=O)c5ccc(F)cc5)CC4)cc3F)c12] (S)-2-Amino-3-(3-fluoro-4-((5-methyl-7H- pyrrolo[2,3-d]pyrimidin-4-yl)oxy)phenyl)-1-(4- ((4-fluorophenyl)sulfonyl)piperidin-1-yl)propan- 1-one | 1E-b/4-((4- fluorophenyl)- sulfonyl)-piperidine | ¹H NMR (400 MHz, DMSO) δ 11.90 (s, 1H), 8.21-8.16 (m, 1H), 7.95-7.89 (m, 2H), 7.57-7.49 (m, 2H), 7.32-7.22 (m, 3H), 7.07 (d, J = 7.8 Hz, 1H), 4.50-4.46 (m, 1H), 4.05-4.02 (m, 2H), 3.62-3.53 (m, 2H), 3.04-2.80 (m, 3H), 2.73-2.64 (m, 1H), 2.57-2.52 (m, 1H), 2.44-2.40 (m, 3H), 1.91-1.76 (m, 2H), 1.60-1.49 (m, 1H), 1.37-1.27 (m, 1H). | Rt = 3.20 min, m/z 556.2 [M + H]⁺ (Method 1) |
| 38 | 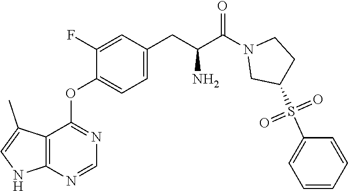 [SMILES: Cc1c[nH]c2ncnc(Oc3ccc(C[C@H](N)C(=O)N4CC[C@H](S(=O)(=O)c5ccccc5)C4)cc3F)c12] (S)-2-Amino-3-(3-fluoro-4-((5-methyl-7H- pyrrolo[2,3-d]pyrimidin-4-yl)oxy)phenyl)-1-((S)- 3-(phenylsulfonyl)pyrrolidin-1-yl)propan-1-one | 1E-b/(S)-3- (phenylsulfonyl) pyrrolidine | ¹H NMR (400 MHz, DMSO) δ 11.91 (s, 1H), 8.15-8.15 (m, 1H), 7.93-7.89 (m, 2H), 7.83-7.77 (m, 1H), 7.72-7.67 (m, 2H), 7.33-7.21 (m, 3H), 7.12-7.05 (m, 1H), 4.30-4.06 (m, 1H), 3.96-3.59 (m, 3H), 3.51-3.34 (m, 1H), 2.88-2.76 (m, 1H), 2.68-2.60 (m, 1H), 2.43-2.40 (m, 3H), 2.26-2.07 (m, 3H), 1.74 (s, 2H). | Rt = 3.04 min, m/z 524.2 [M + H]⁺ (Method 1) |
| 39 | 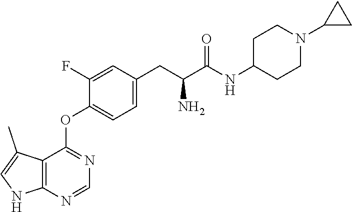 [SMILES: Cc1c[nH]c2ncnc(Oc3ccc(C[C@H](N)C(=O)NC4CCN(C5CC5)CC4)cc3F)c12] (S)-2-Amino-N-(1-cyclopropylpiperidin-4-yl)-3- (3-fluoro-4-((5-methyl-7H-pyrrolo[2,3- d]pyrimidin-4-yl)oxy)phenyl)propanamide | 1E-b/1-cyclopropyl- piperidin-4-amine | ¹H NMR (400 MHz, DMSO) δ 11.90 (s, 1H), 8.19 (s, 1H), 7.67 (d, J = 7.8 Hz, 1H), 7.29 (dd, J = 8.2, 8.2 Hz, 1H), 7.24- 7.17 (m, 2H), 7.08 (dd, J = 1.3, 8.3 Hz, 1H), 3.57-3.47 (m, 1H), 3.42-3.36 (m, 1H), 2.92-2.75 (m, 3H), 2.69 (dd, J = 7.7, 13.3 Hz, 1H), 2.42 (d, J = 0.9 Hz, 3H), 2.25-2.14 (m, 2H), 1.78 (s, 2H), 1.67- 1.52 (m, 3H), 1.36-1.21 (m, 2H), 0.42- 0.35 (m, 2H), 0.29-0.23 (m, 2H). | Rt = 2.16 min, m/z 453.2 [M + H]⁺ (Method 1) |

### Example 40

Step A

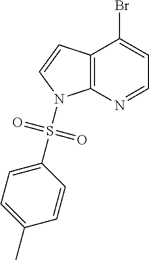 [SMILES: Cc1ccc(S(=O)(=O)n2ccc3c(Br)ccnc32)cc1]

### 4-Bromo-1-tosyl-1H-pyrrolo[2,3-b]pyridine (Intermediate 40A)

4-Bromo-7-azaindole (5.0 g, 28.90 mmol) was dissolved in DMF (40 mL) and the solution was stirred at RT under a stream of nitrogen. Sodium hydride (60% on mineral oil, 1.50 g, 37.58 mmol) was added portion wise and the reaction was stirred for 30 min. A solution of 4-toluenesulfonyl chloride (5.77 g, 30.37 mmol) in DMF (10 mL) was added dropwise over 10 min, and then the reaction was stirred for a further 2 h. The reaction mixture was carefully poured into cold water (100 mL) and stirred for 30 min. The resulting precipitate was collected by filtration and dried in vacuo to afford the compound (9.12 g).

LCMS (Method 7): Rt=1.59 min, m/z 351.1/353.1 [M+H]⁺

Step B

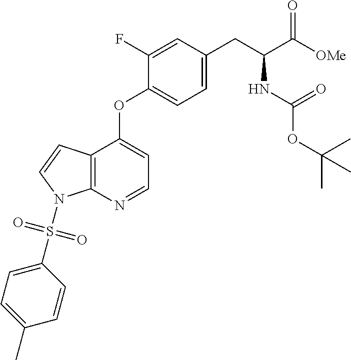 [SMILES: COC(=O)[C@H](Cc1ccc(Oc2ccnc3c2ccn3S(=O)(=O)c2ccc(C)cc2)c(F)c1)NC(=O)OC(C)(C)C]

### Methyl (S)-2-((tert-butoxycarbonyl)amino)-3-(3-fluoro-4-((1-tosyl-1H-pyrrolo[2,3-b]pyridin-4-yl)oxy)phenyl)propanoate (Intermediate 40B)

A solution of Intermediate 1B-a (0.47 g, 1.49 mmol), Intermediate 40A (0.5 g, 1.42 mmol), Pd₂(dba)₃ (0.065 g, 0.071 mmol), XPhos (0.068 g, 0.142 mmol), potassium carbonate (0.59 g, 4.27 mmol) in toluene (10 mL) was stirred at 95° C. for 24 h. The reaction mixture was filtered through Celite®. The solution, diluted with ethyl acetate (50 mL) was washed with water (50 mL). The product was extracted into ethyl acetate (2×50 mL). The combined extracts were dried (Na₂SO₄) and evaporated. The residue was chromatographed on a 25 g Si cartridge eluting with 0-100% ethyl acetate in isohexane to give Intermediate 40B (0.273 g).

LCMS (Method 9): Rt=1.70 min, m/z 584.3 [M+H]⁺

Step C

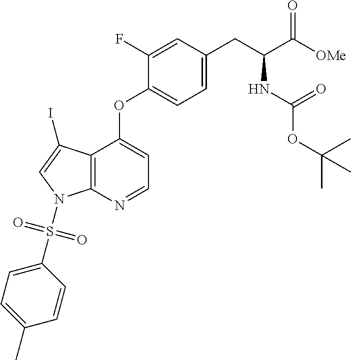 [SMILES: COC(=O)[C@H](Cc1ccc(Oc2ccnc3c2c(I)cn3S(=O)(=O)c2ccc(C)cc2)c(F)c1)NC(=O)OC(C)(C)C]

### Methyl (S)-2-((tert-butoxycarbonyl)amino)-3-(3-fluoro-4-((3-iodo-1-tosyl-1H-pyrrolo[2,3-b]pyridin-4-yl)oxy)phenyl)propanoate (Intermediate 40C)

NIS (0.11 g, 0.49 mmol) was added portionwise to an ice-cooled solution of Intermediate 40B (0.273 g, 0.47 mmol) in MeCN (10 mL) and the resulting mixture was allowed to warm to RT and left to stir for 3 h followed by 2 h at 50° C., then after having added another portion of NIS (0.33 g, 1.47 mmol), the reaction mixture was stirred for a further 2 days at 80° C. The reaction was quenched by addition of aqueous sodium metabisulfite solution (1M) and the resulting mixture was extracted with ethyl acetate (×3). The ethyl acetate layers were separated, combined, dried (Na₂SO₄) and evaporated under reduced pressure. The residue was chromatographed on a 10 g Si cartridge eluting with 0-100% ethyl acetate in isohexane to give the desired product (0.16 g).

LCMS (Method 9): Rt=1.68 min, m/z 710.2 [M+H]⁺

Step D

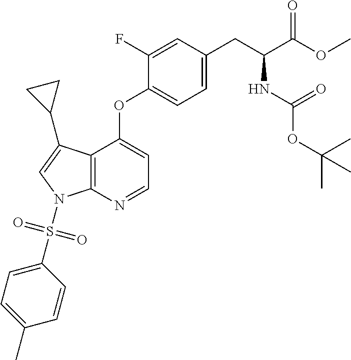 [SMILES: COC(=O)[C@H](Cc1ccc(Oc2ccnc3c2c(C2CC2)cn3S(=O)(=O)c2ccc(C)cc2)c(F)c1)NC(=O)OC(C)(C)C]

### Methyl (S)-2-((tert-butoxycarbonyl)amino)-3-(4-((3-cyclopropyl-1-tosyl-1H-pyrrolo[2,3-b]pyridin-4-yl)oxy)-3-fluorophenyl)propanoate (Intermediate 40D)

A mixture of intermediate 40C (0.50 g, 0.71 mmol), cyclopropylboronic acid (0.15 g, 1.76 mmol), Pd(dppf)Cl₂.CH₂Cl₂ (0.029 g, 0.035 mmol) and potassium carbonate (0.29 g, 2.11 mmol) in DMF (5 mL) was sonicated for 5 min under a blanket of argon. The mixture was heated at 100° C. for 5 h, and then allowed to cool to RT before diluting with water. The mixture was extracted with ethyl acetate and the organic phase was dried (Na₂SO₄) and evaporated. The crude product was chromatographed on a 40 g Si cartridge eluting with 0-100% ethyl acetate in isohexane to give the desired product (0.17 g).

LCMS (Method 9): Rt=1.68 min, m/z 624.3 [M+H]⁺

Step E

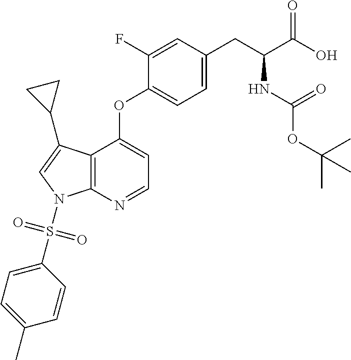 [SMILES: Cc1ccc(S(=O)(=O)n2cc(C3CC3)c3c(Oc4ccc(C[C@H](NC(=O)OC(C)(C)C)C(=O)O)cc4F)ccnc32)cc1]

### (S)-2-((tert-Butoxycarbonyl)amino)-3-(4-((3-cyclopropyl-1-tosyl-1H-pyrrolo[2,3-b]pyridin-4-yl)oxy)-3-fluorophenyl)propanoic Acid (Intermediate 40E)

Intermediate 40D (0.21 g, 0.34 mmol) was dissolved in a mixture of methanol (1.7 mL), water (1.7 mL) and THF (1 mL). Lithium hydroxide hydrate (0.014 g, 0.34 mmol) was added and the reaction mixture was stirred at RT for 5 h. The solvent was reduced and the product was extracted into ethyl acetate (×2). The combined organic extracts were washed with brine, dried (Na₂SO₄) and evaporated to give the title compound (0.15 g).

LCMS (Method 9): Rt=1.61 min, m/z 610.1 [M+H]⁺

Step F

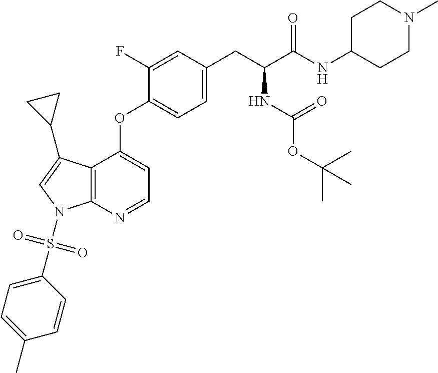 [SMILES: Cc1ccc(S(=O)(=O)n2cc(C3CC3)c3c(Oc4ccc(C[C@H](NC(=O)OC(C)(C)C)C(=O)NC5CCN(C)CC5)cc4F)ccnc32)cc1]

### tert-Butyl (S)-(3-(4-((3-cyclopropyl-1-tosyl-1H-pyrrolo[2,3-b]pyridin-4-yl)oxy)-3-fluorophenyl)-1-((1-methylpiperidin-4-yl)amino)-1-oxopropan-2-Yl)carbamate (Intermediate 40F)

Intermediate 40F was prepared from Intermediate 40E and 1-methylpiperidin-4-amine using a similar procedure to that used in Step F of Example 1.

LCMS (Method 9): Rt=1.19 min, m/z 706.3 [M+H]⁺

Step G

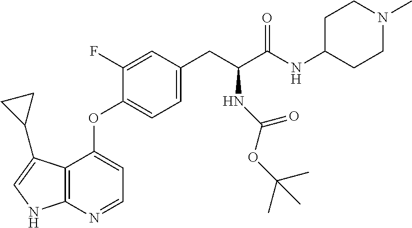 [SMILES: CN1CCC(NC(=O)[C@H](Cc2ccc(Oc3ccnc4[nH]cc(C5CC5)c34)c(F)c2)NC(=O)OC(C)(C)C)CC1]

### tert-Butyl (S)-(3-(4-((3-cyclopropyl-1H-pyrrolo[2,3-b]pyridin-4-yl)oxy)-3-fluorophenyl)-1-((1-methylpiperidin-4-yl)amino)-1-oxopropan-2-yl)carbamate (Intermediate 40G)

Lithium hydroxide monohydrate (20 mg, 0.48 mmol) was added to a solution of Intermediate 40F (113 mg, 0.16 mmol) in a mixture of methanol (0.7 mL), water (0.7 mL) and THF (0.4 mL) and the resulting mixture was stirred at RT for 18 h and then at 60° C. for 2 h. The solvent was removed under reduced pressure and the residue was diluted with water and extracted with ethyl acetate (×3). The ethyl acetate layers were separated, combined, dried (Na₂SO₄) and evaporated under reduced pressure to give the desired product (71 mg) that was used in the next step without further purification.

LCMS (Method 9): Rt=1.04 min, m/z 552.3 [M+H]⁺

Step H

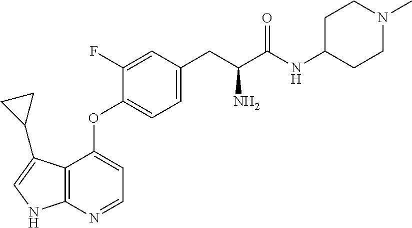 [SMILES: CN1CCC(NC(=O)[C@@H](N)Cc2ccc(Oc3ccnc4[nH]cc(C5CC5)c34)c(F)c2)CC1]

### (S)-2-Amino-3-(4-((3-cyclopropyl-1H-pyrrolo[2,3-b]pyridin-4-yl)oxy)-3-fluorophenyl)-N-(1-methylpiperidin-4-yl)propanamide (Example 40)

Intermediate 40G (71 mg, 0.13 mmol) was dissolved in a mixture of DCM (1.0 mL) and TFA (0.2 mL), and the reaction was stirred at RT for 1 h. The mixture was diluted with methanol and passed down a 5 g SCX-2 cartridge eluting with methanol and then 3.5M methanolic ammonia. The ammonia solution was evaporated to give a residue which was purified by MDAP using an Xbridge Phenyl column (19×150 mm, 10 μm particle size) and eluting with 40-60% MeOH/H₂O (10 mM NH₄CO₃) to afford the desired compound (13 mg).

LCMS (Method 2): Rt=1.91 min, m/z 452.1 [M+H]⁺

¹H NMR (400 MHz, DMSO) δ 11.42 (s, 1H), 7.98 (d, J=5.4 Hz, 1H), 7.65 (d, J=7.9 Hz, 1H), 7.28-7.20 (m, 2H), 7.10-7.04 (m, 2H), 6.18 (d, J=5.4 Hz, 1H), 3.51-3.46 (m, 1H), 3.37 (dd, J=6.7, 6.7 Hz, 1H), 2.86 (dd, J=5.9, 13.3 Hz, 1H), 2.74-2.52 (m, 3H), 2.12 (s, 3H), 2.11-2.07 (m, 1H), 1.94-1.88 (m, 2H), 1.66-1.56 (m, 2H), 1.42-1.27 (m, 2H), 0.78 (ddd, J=3.9, 6.0, 8.3 Hz, 2H), 0.63-0.58 (m, 2H).

ee=36%

### Example 41

Step A

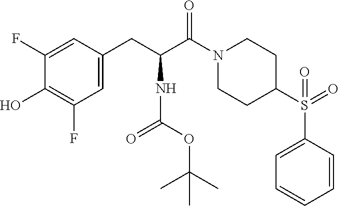 [SMILES: CC(C)(C)OC(=O)N[C@@H](Cc1cc(F)c(O)c(F)c1)C(=O)N1CCC(S(=O)(=O)c2ccccc2)CC1]

### tert-Butyl (S)-(3-(3,5-difluoro-4-hydroxyphenyl)-1-oxo-1-(4-(phenylsulfonyl)-piperidin-1-yl)propan-2-yl)carbamate (Intermediate 41A)

A mixture of 4-(phenylsulfonyl)piperidine hydrochloride (1.0 g, 3.8 mmol), (S)-2-((tert-butoxycarbonyl)amino)-3-(3,5-difluoro-4-hydroxyphenyl)propanoic acid (1.1 g, 3.5 mmol), DIPEA (1.8 mL, 10.4 mmol), DCM (16 mL) and DMF (4 mL) was treated with HATU (1.59 g, 4.2 mmol). The mixture was stirred for 1 h then the resulting solution was allowed to stand for 18 h. The mixture was diluted with dichloromethane (30 mL) and washed with saturated sodium hydrogen carbonate (aq.) (20 mL) then saturated brine (2×20 mL). The organic phase was dried (Na₂SO₄) and concentrated in vacuo and purified by chromatography on a Si cartridge eluting with 0-20% DCM in ethyl acetate to give Intermediate 41A (780 mg).

LCMS (Method 3): Rt=1.25 min. m/z 525.3 [M+H]⁺

Step B

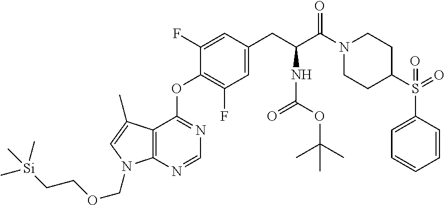 [SMILES: Cc1cn(COCC[Si](C)(C)C)c2ncnc(Oc3c(F)cc(C[C@H](NC(=O)OC(C)(C)C)C(=O)N4CCC(S(=O)(=O)c5ccccc5)CC4)cc3F)c12]

### tert-Butyl (S)-(3-(3,5-difluoro-4-((5-methyl-7-((2-(trimethylsilyl)ethoxy)-methyl)-7H-pyrrolo[2,3-d]pyrimidin-4-yl)oxy)phenyl)-1-oxo-1-(4-(phenylsulfonyl)-piperidin-1-yl)propan-2-yl)carbamate (Intermediate 41B)

A mixture of Intermediate 41A (315 mg, 0.60 mmol), Intermediate 1C-b (268 mg, 0.9 mmol) and potassium carbonate (249 mg, 1.80 mmol) in DMSO (6 mL) was stirred and heated at 110° C. for 2 h. The mixture was cooled, diluted with ethyl acetate (30 mL) and washed with a mixture of water (20 mL) and saturated brine (5 mL). The aqueous phase was washed with ethyl acetate (10 mL). The combined organic phase was washed with saturated brine, dried (MgSO₄) and concentrated in vacuo. The residue was purified by flash chromatography on a 5 g Si cartridge eluting with 0-50% DCM in ethyl acetate to afford the desired product (320 mg).

LCMS (Method 9): Rt=1.81 min. m/z 786.4 [M+H]⁺

Step C

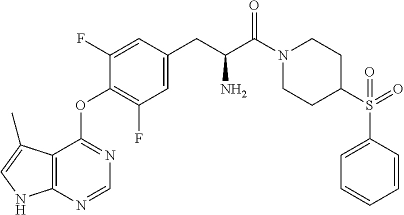 [SMILES: Cc1c[nH]c2ncnc(Oc3c(F)cc(C[C@H](N)C(=O)N4CCC(S(=O)(=O)c5ccccc5)CC4)cc3F)c12]

### (S)-2-Amino-3-(3,5-difluoro-4-((5-methyl-7H-pyrrolo[2,3-d]pyrimidin-4-yl)oxy)phenyl)-1-(4-(phenylsulfonyl)piperidin-1-yl)propan-1-one (Example 41)

Example 41 was prepared from Intermediate 41B according to Step G of Example 1.

LCMS (Method 1) 3.13 mins, m/z 556.2 [M+H]⁺

¹H NMR (400 MHz, DMSO) δ 12.01 (s, 1H), 8.22 (apparent d J=10.9 Hz, 1H), 7.85 (d J=7.7 Hz, 2H), 7.82-7.75 (m, 1H), 7.73-7.65 (m, 2H), 7.27 (s, 1H), 7.16 (d J=9.0 Hz, 2H), 4.48 (d J=12.5 Hz, 2H), 4.16-4.05 (m, 1H), 3.96-3.88 (m, 1H), 3.63-3.50 (m, 1H), 3.06-2.88 (m, 1H), 2.84-2.76 (m, 1H), 2.70-2.50 (m), 2.43 (s) and 2.42 (s) (together 3H), 1.91-1.63 (m, 3H), 01.63-1.18 (m, 2H).

### Example 42

Step A

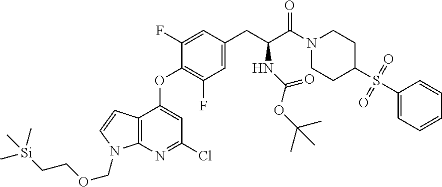 [SMILES: CC(C)(C)OC(=O)N[C@@H](Cc1cc(F)c(Oc2cc(Cl)nc3c2ccn3COCC[Si](C)(C)C)c(F)c1)C(=O)N1CCC(S(=O)(=O)c2ccccc2)CC1]

### tert-Butyl (S)-(3-(4-((6-chloro-1-((2-(trimethylsilyl)ethoxy)methyl)-1H-pyrrolo[2,3-b]pyridin-4-yl)oxy)-3,5-difluorophenyl)-1-oxo-1-(4-(phenyl-sulfonyl)piperidin-1-yl)propan-2-yl)carbamate (Intermediate 42A)

A solution of Intermediate 1C-c (0.27 g, 0.813 mmol), Intermediate 41A (0.64 g, 1.22 mmol) and potassium carbonate (0.34 g, 1.56 mmol) in DMSO (5 mL) was stirred at 120° C. for 2 h. The reaction mixture was then partitioned between ethyl acetate (3×30 mL) and water (20 mL). The organic layer was washed with brine (20 mL) dried over sodium sulfate and evaporated in vacuo. The residue was chromatographed on a Si cartridge eluting with 0-70% ethyl acetate in isohexane to give Intermediate 42A (460 mg).

LCMS (Method 9): Rt=1.80 min, m/z 805.4/807.4 [M+H]⁺

Step B

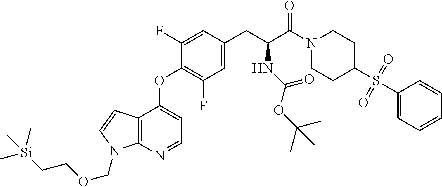 [SMILES: CC(C)(C)OC(=O)N[C@@H](Cc1cc(F)c(Oc2ccnc3c2ccn3COCC[Si](C)(C)C)c(F)c1)C(=O)N1CCC(S(=O)(=O)c2ccccc2)CC1]

### tert-Butyl (S)-(3-(3,5-difluoro-4-((1-((2-(trimethylsilyl)ethoxy)methyl)-1H-pyrrolo[2,3-b]pyridin-4-yl)oxy)phenyl)-1-oxo-1-(4-(phenylsulfonyl)piperidin-1-yl)propan-2-yl)carbamate (Intermediate 42B)

A solution of Intermediate 42A (460 mg, 0.57 mmol) and trimethylamine (0.096 mL, 0.69 mmol) in IMS (20 mL) was stirred at RT over 10% palladium on carbon (50 mg) under a blanket of hydrogen gas. After 4 days a second aliquot of 10% palladium on carbon (50 mg) was added. Stirring was continued for a total of 10 days then the mixture was filtered through Celite® and the solvent was evaporated to give the crude product. This was further purified by flash chromatography using a 5 g silica cartridge eluting with 0-50% ethyl acetate in DCM to afford the desired product (410 mg).

LCMS (Method 9): Rt=1.67 min, m/z 771.4 [M+H]⁺

Step C

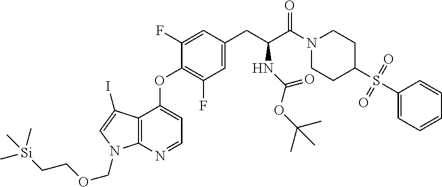 [SMILES: CC(C)(C)OC(=O)N[C@@H](Cc1cc(F)c(Oc2ccnc3c2c(I)cn3COCC[Si](C)(C)C)c(F)c1)C(=O)N1CCC(S(=O)(=O)c2ccccc2)CC1]

### tert-Butyl (S)-(3-(3,5-difluoro-4-((3-iodo-1-((2-(trimethylsilyl)ethoxy)-methyl)-1H-pyrrolo[2,3-b]pyridin-4-yl)oxy)phenyl)-1-oxo-1-(4-(phenylsulfonyl)-piperidin-1-yl)propan-2-yl)carbamate (Intermediate 42C)

NIS (0.13 g, 0.578 mmol) was added to a solution of Intermediate 42B (0.405 g, 0.525 mmol) in MeCN (10 mL) at 0° C. and the resulting mixture was stirred at RT for 16 h. The reaction was quenched by addition of saturated aqueous sodium sulfite solution (30 mL) and stirred for 30 mins. The resulting mixture was extracted with DCM (×2). The DCM layers were washed with brine (20 mL), combined, dried (Na₂SO₄) and evaporated under reduced pressure to give Intermediate 42C (0.39 g) that used in next step without further purification.

LCMS (Method 9): Rt=1.75 min, m/z 897.3 [M+H]⁺

Step D

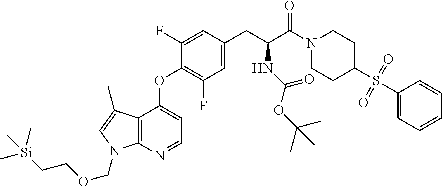 [SMILES: Cc1cn(COCC[Si](C)(C)C)c2nccc(Oc3c(F)cc(C[C@H](NC(=O)OC(C)(C)C)C(=O)N4CCC(S(=O)(=O)c5ccccc5)CC4)cc3F)c12]

### tert-Butyl (S)-(3-(3,5-difluoro-4-((3-methyl-1-((2-(trimethylsilyl)ethoxy)-methyl)-1H-pyrrolo[2,3-b]pyridin-4-yl)oxy)phenyl)-1-oxo-1-(4-(phenylsulfonyl)-piperidin-1-yl)propan-2-yl)carbamate (Intermediate 42D)

Intermediate 42C (0.39 g, 0.435 mmol), potassium phosphate tribasic (0.277 g, 1.30 mmol) and SPhosPdG2 (0.047 g, 0.0652 mmol) were dissolved in THF (9 mL) and purged with nitrogen for 5 min, then 2,4,6-trimethyl-1,3,5,2,4,6-trioxatriborinane (0.37 mL, 1.30 mmol) and water (3 mL) were added and the mixture purged for further 2 min prior to heating in a microwave reactor at 80° C. for 1 h. The cold mixture was partitioned between water (20 mL) and ethyl acetate (2×40 mL). The combined organic phase was washed with water (2×20 mL), HCl (0.5M) (2×20 mL), brine (2×20 mL), dried (Na₂SO₄) and evaporated. The residue was chromatographed on a Si cartridge eluting with 0-100% ethyl acetate in isohexane to give the title compound (200 mg).

LCMS (Method 9): Rt=1.75 min, m/z 785.4 [M+H]⁺

Step E

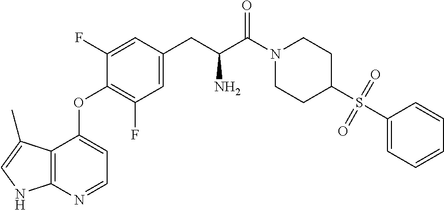 [SMILES: Cc1c[nH]c2nccc(Oc3c(F)cc(C[C@H](N)C(=O)N4CCC(S(=O)(=O)c5ccccc5)CC4)cc3F)c12]

### (S)-2-amino-3-(3,5-difluoro-4-((3-methyl-1H-pyrrolo[2,3-b]pyridin-4-v)oxy)phenyl)-1-(4-(phenylsulfonyl)piperidin-1-yl)propan-1-one (Example 42)

Intermediate 42D (0.2 g, 0.255 mmol) was dissolved in a mixture of DCM (10 mL) and TFA (10 mL), and the reaction mixture was stirred at RT for 1 h. The mixture was passed down a 20 g SCX-2 cartridge eluting with DCM, methanol and then 2M methanolic ammonia. After standing for 18 h, the ammonia solution was evaporated to give a light yellow residue (170 mg). The crude material was purified by MDAP using an Xbridge Phenyl column (19×150 mm, 10 μm particle size) and eluting with 20-80% MeOH/H₂O (10 mM NH₄CO₃) to give the title compound.

LCMS (Method 2): Rt=2.71 min, m/z 555.3[M+H]⁺

¹H NMR (400 MHz, DMSO) δ 11.43 (s, 1H), 8.02-7.93 (m, 1H), 7.88-7.82 (m, 2H), 7.81-7.74 (m, 1H), 7.73-7.64 (m, 2H), 7.21 (d J=9.13 Hz, 2H), 7.16 (s, 1H), 6.18-6.09 (m, 1H), 4.55-4.39 (m, 1H), 4.20-4.02 (m, 1H), 3.98-3.84 (m, 1H), 3.67-3.48 (m, 1H), 3.08-2.87 (m, 1H), 2.87-2.74 (m, 1H), 2.72-2.52 (m, 2H), 2.44 (s) and 2.42 (s) (together 3H), 1.93-1.64 (m, 4H), 1.42-1.15 (m, 2H).

### Example 43

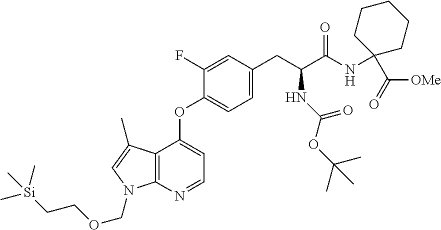 [SMILES: COC(=O)C1(NC(=O)[C@H](Cc2ccc(Oc3ccnc4c3c(C)cn4COCC[Si](C)(C)C)c(F)c2)NC(=O)OC(C)(C)C)CCCCC1]

### Methyl (S)-1-(2-((tert-butoxycarbonyl)amino)-3-(3-fluoro-4-((3-methyl-1-((2-(trimethylsilyl)ethoxy)methyl)-1H-pyrrolo[2,3-b]pyridin-4-yl)oxy)phenyl)-propanamido)cyclohexane-1-carboxylate (Intermediate 43A)

Intermediate 1E-a (100 mg, 0.18 mmol), methyl 1-aminocyclohexane-1-carboxylate (31 mg, 0.20 mmol) and COMU (92 mg, 0.21 mmol) were dissolved in DCM (3.0 mL) and DIPEA (0.068 mL, 0.39 mmol) was added. The reaction was stirred at RT for 1.5 h. A further amount of methyl 1-aminocyclohexane-1-carboxylate (7 mg, 0.045 mmol), COMU (19 mg, 0.045 mmol) and DIPEA (0.017 ml, 0.098 mmol) were added and the resulting mixture was stirred for a further 30 min. Water was added and the DCM layer was separated. The aqueous was further extracted with DCM (×2) and the combined organic extracts were dried (Na₂SO₄) and evaporated. The product was purified by chromatography on a Si cartridge eluting with 0-60% ethyl acetate in cyclohexane to give Intermediate 43A (79 mg).

LCMS (Method 9): Rt=1.82 min, m/z 699.3 [M+H]⁺

Step B

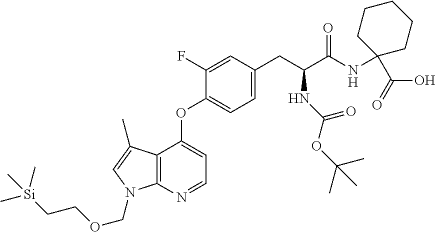 [SMILES: Cc1cn(COCC[Si](C)(C)C)c2nccc(Oc3ccc(C[C@H](NC(=O)OC(C)(C)C)C(=O)NC4(C(=O)O)CCCCC4)cc3F)c12]

### (S)-1-(2-((Tert-butoxycarbonyl)amino)-3-(3-fluoro-4-((3-methyl-1-((2-(trimethylsilyl)ethoxy)methyl)-1H-pyrrolo[2,3-b]pyridin-4-yl)oxy)phenyl)-propanamido)cyclohexane-1-carboxylic Acid (Intermediate 43B)

Intermediate 43A (79 mg, 0.11 mmol) was dissolved in a mixture of methanol (0.6 mL), water (0.6 mL) and THF (0.3 mL). Lithium hydroxide hydrate (14 mg, 0.34 mmol) was added and the reaction mixture was stirred at RT for 2 h. A further amount of lithium hydroxide hydrate (14 mg, 0.34 mmol) was added and the reaction mixture was stirred at RT for 18 h. A further amount of lithium hydroxide hydrate (14 mg, 0.34 mmol) was added and the reaction mixture was stirred at RT for 18 h. The solvent was reduced and the product was extracted into ethyl acetate (×2). The combined organic extracts were washed with brine, dried (Na₂SO₄) and evaporated to give the desired product (68 mg).

LCMS (Method 9): Rt=1.73 min, m/z 685.4 [M+H]⁺

Step C

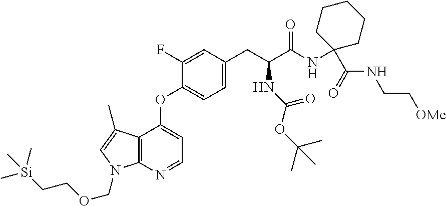 [SMILES: COCCNC(=O)C1(NC(=O)[C@H](Cc2ccc(Oc3ccnc4c3c(C)cn4COCC[Si](C)(C)C)c(F)c2)NC(=O)OC(C)(C)C)CCCCC1]

### (tert-Butyl (S)-(3-(3-fluoro-4-((3-methyl-1-((2-(trimethylsilyl)ethoxy)methyl)-1H-pyrrolo[2,3-b]pyridin-4-yl)oxy)phenyl)-1-((1-((2-methoxyethyl)carbamoyl)-cyclohexyl)amino)-1-oxopropan-2-yl)carbamate (Intermediate 43C)

Intermediate 43B (68 mg, 0.099 mmol), 2-methoxyethylamine (8.2 mg, 0.11 mmol) and COMU (51 mg, 0.12 mmol) were dissolved in DCM (1.7 mL) and DIPEA (0.038 mL, 0.22 mmol) was added. The reaction was stirred at RT for 3.5 h. A further amount of 2-methoxyethylamine (8.2 mg, 0.11 mmol), COMU (51 mg, 0.12 mmol) and DIPEA (0.038 ml, 0.22 mmol) were added and the resulting mixture was stirred for a further 2 h. Water was added and the DCM layer was separated. The aqueous was further extracted with DCM (×2) and the combined organic extracts were dried (Na₂SO₄) and evaporated. The product was purified by chromatography on a Si cartridge eluting with 0-100% ethyl acetate in cyclohexane to give Intermediate 43C (28 mg).

LCMS (Method 9): Rt=1.72 min, m/z 742.4 [M+H]⁺

Step D

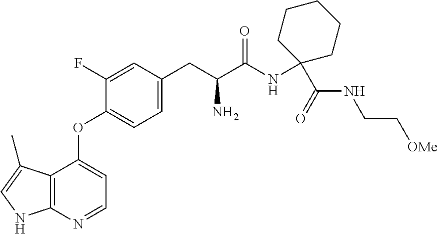 [SMILES: COCCNC(=O)C1(NC(=O)[C@@H](N)Cc2ccc(Oc3ccnc4[nH]cc(C)c34)c(F)c2)CCCCC1]

### (S)-1-(2-amino-3-(3-fluoro-4-((3-methyl-1H-pyrrolo[2,3-b]pyridin-4-yl)oxy)phenyl)propanamido)-N-(2-methoxyethyl)cyclohexanecarboxamide (Example 43)

Example 43 was prepared from Intermediate 43C using a method analogous to that used in Step G of Example 1.

LCMS: Rt=2.59 min, m/z 512.3 [M+H]⁺ (Method 1)

¹H NMR (400 MHz, DMSO) δ 11.40 (d, J=1.3 Hz, 1H), 7.98 (d, J=5.4 Hz, 1H), 7.69 (s, 1H), 7.40 (dd, J=5.7, 5.7 Hz, 1H), 7.33 (dd, J=1.9, 12.0 Hz, 1H), 7.24 (dd, J=8.4, 8.4 Hz, 1H), 7.16-7.13 (m, 2H), 6.17 (d, J=5.4 Hz, 1H), 3.53 (dd, J=5.7, 7.8 Hz, 1H), 3.29-3.25 (m, 2H), 3.22 (s, 3H), 3.21-3.14 (m, 2H), 2.96 (dd, J=5.6, 13.5 Hz, 1H), 2.73-2.66 (m, 1H), 2.38 (d, J=1.0 Hz, 3H), 2.03-1.89 (m, 3H), 1.66-1.56 (m, 2H), 1.48-1.45 (m, 3H), 1.33 (d, J=12.2 Hz, 1H), 1.19-1.14 (m, 1H).

### Example 44

Step A

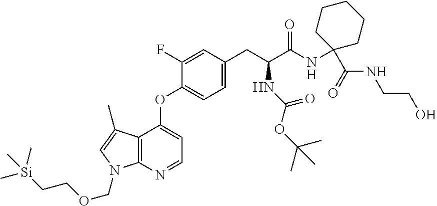 [SMILES: Cc1cn(COCC[Si](C)(C)C)c2nccc(Oc3ccc(C[C@H](NC(=O)OC(C)(C)C)C(=O)NC4(C(=O)NCCO)CCCCC4)cc3F)c12]

### tert-Butyl (S)-(3-(3-fluoro-4-((3-methyl-1-((2-(trimethylsilyl)ethoxy)methyl)-1H-pyrrolo[2,3-b]pyridin-4-yl)oxy)phenyl)-1-((1-((2-hydroxyethyl)carbamoyl)-cyclohexyl)amino)-1-oxopropan-2-yl)carbamate (Intermediate 44A)

Intermediate 43B (138 mg, 0.20 mmol) was dissolved in DMF (1.0 mL) and cooled in an ice bath. TBTU (129 mg, 0.40 mmol), HOBt (54 mg, 0.40 mmol) and DIPEA (0.11 mL, 0.60 mmol) were added and the resulting mixture was stirred for 10 minutes. Ethanolamine (0.015 mL, 0.24 mmol) was added and the mixture was allowed to warm to room temperature and stirred for 5 h. Water and ethyl acetate were added and the organic layer was separated. The aqueous was further extracted with ethyl acetate (×2) and the combined organic extracts were dried (Na₂SO₄) and evaporated. The product was purified by chromatography on a 25 g Si cartridge eluting with 0-100% ethyl acetate in cyclohexane to give Intermediate 44A (68 mg).

LCMS (U1152340): Rt=1.63 min, m/z 728.4 [M+H]⁺

ee %=73%

Step B

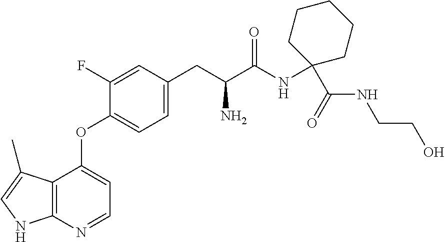 [SMILES: Cc1c[nH]c2nccc(Oc3ccc(C[C@H](N)C(=O)NC4(C(=O)NCCO)CCCCC4)cc3F)c12]

### (S)-1-(2-amino-3-(3-fluoro-4-((3-methyl-1H-pyrrolo[2,3-b]pyridin-4-yl)oxy)phenyl)propanamido)-N-(2-hydroxyethyl)cyclohexanecarboxamide (Example 44)

Example 44 was prepared from Intermediate 44A using the conditions outlined in Step G of Example 1.

LCMS (Method 1): Rt=2.47 min, m/z 498.3 [M+H]⁺

¹H NMR (400 MHz, DMSO) δ 11.39 (s, 1H), 7.98 (d, J=5.4 Hz, 1H), 7.73 (s, 1H), 7.38-7.31 (m, 2H), 7.24 (dd, J=8.4, 8.4 Hz, 1H), 7.15-7.12 (m, 2H), 6.18-6.16 (m, 1H), 4.52 (s, 1H), 3.54 (dd, J=5.9, 7.8 Hz, 1H), 3.16-3.03 (m, 2H), 2.95 (dd, J=5.8, 13.5 Hz, 1H), 2.74-2.66 (m, 1H), 2.38 (d, J=1.0 Hz, 3H), 2.09-1.83 (m, 3H), 1.67-1.56 (m, 2H), 1.48-1.45 (m, 3H), 1.36-1.31 (m, 1H), 1.15 (dd, J=9.2, 9.2 Hz, 2H).

### Preparation of Examples 45 to 47

The following examples were prepared in a similar manner to Example 1, following the same synthetic sequence, by replacing in Step F the indicated amine starting materials in the table below and using 5 equivalents of DIPEA.

| Ex | Structure/Name | Amine | 1H NMR | LC-MS |
|---|---|---|---|---|
| 45 | 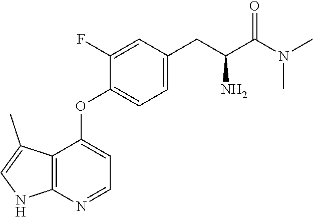 [SMILES: Cc1c[nH]c2nccc(Oc3ccc(C[C@H](N)C(=O)N(C)C)cc3F)c12] (S)-2-amino-3-(3-fluoro-4-((3-methyl-1H-pyrrolo[2,3- b]pyridin-4-yl)oxy)phenyl)-N,N-dimethylpropanamide ee % (n.d.) | Dimethylamine hydrochloride | ¹H NMR (400 MHz, DMSO) δ 11.39 (s, 1H), 7.99 (d, J = 5.4 Hz, 1H), 7.32 (dd, J = 1.9, 11.9 Hz, 1H), 7.26-7.20 (m, 1H), 7.14-7.10 (m, 2H), 6.14 (dd, J = 0.8, 5.5 Hz, 1H), 3.89 (dd, J = 5.9, 7.8 Hz, 1H), 2.94 (s, 3H), 2.82-2.81 (m, 4H), 2.63 (dd, J = 7.8, 13.3 Hz, 1H), 2.38 (d, J = 1.0 Hz, 3H), 1.73 (s, 2H). | Rt = 2.11 min, m/z 357.3 [M + H]⁺ (Method 1) |
| 46 | 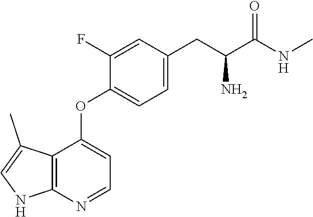 [SMILES: CNC(=O)[C@@H](N)Cc1ccc(Oc2ccnc3[nH]cc(C)c23)c(F)c1] (S)-2-amino-3-(3-fluoro-4-((3-methyl-1H-pyrrolo[2,3- b]pyridin-4-yl)oxy)phenyl)-N-methylpropanamide ee % (n.d.) | Methylamine hydrochloride | ¹H NMR (400 MHz, DMSO) δ 11.39 (s, 1H), 7.98 (d, J = 5.4 Hz, 1H), 7.83-7.78 (m, 1H), 7.30-7.21 (m, 2H), 7.14-7.07 (m, 2H), 6.17 (d, J = 5.4 Hz, 1H), 3.37 (dd, J = 5.1, 8.2 Hz, 1H), 2.95 (dd, J = 5.0, 13.4 Hz, 1H), 2.66 (dd, J = 8.3, 13.3 Hz, 1H), 2.59 (d, J = 4.7 Hz, 3H), 2.38 (d, J = 1.0 Hz, 3H), 1.83 (s, 2H). | Rt = 2.01 min, m/z 343.3 [M + H]⁺ (Method 1) |
| 47 | 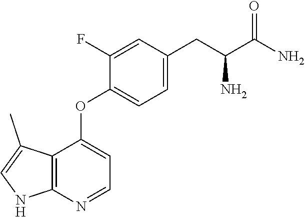 [SMILES: Cc1c[nH]c2nccc(Oc3ccc(C[C@H](N)C(N)=O)cc3F)c12] (S)-2-amino-3-(3-fluoro-4-((3-methyl-1H-pyrrolo[2,3- b]pyridin-4-yl)oxy)-phenyl)propanamide ee % (n.d.) | Ammonium chloride | ¹H NMR (400 MHz, DMSO) δ 11.39 (s, 1H), 7.98 (d, J = 5.4 Hz, 1H), 7.36-7.22 (m, 3H), 7.15-7.10 (m, 2H), 6.98 (s, 1H), 6.17 (d, J = 4.8 Hz, 1H), 3.37 (dd, J = 5.1, 8.2 Hz, 1H), 2.94 (dd, J = 5.0, 13.4 Hz, 1H), 2.67 (dd, J = 8.3, 13.4 Hz, 1H), 2.38 (d, J = 1.0 Hz, 3H), 1.83 (s, 2H). | Rt = 1.90 min, m/z 329.3 [M + H]⁺ (Method 1) |

### Preparation of Examples 8a and 8b

The compound of example 8 (2S)-2-Amino-3-(3-fluoro-4-((3-methyl-1H-pyrrolo[2,3-b]pyridin-4-yl)oxy)phenyl)-N-(1,3,3-trimethylpiperidin-4-yl)propanamide was resolved using the conditions given in the following table, to give the two separated single diastereoisomers:

First Eluting single diastereoisomer (first diastereoisomer) of (2S)-2-amino-3-(3-fluoro-4-((3-methyl-1H-pyrrolo[2,3-b]pyridin-4-yl)oxy)phenyl)-N-(1,3,3-trimethylpiperidin-4-yl)propenamide (example 8a)

Second Eluting single diastereoisomer (second diastereoisomer) of (2S)-2-amino-3-(3-fluoro-4-((3-methyl-1H-pyrrolo[2,3-b]pyridin-4-yl)oxy)phenyl)-N-(1,3,3-trimethylpiperidin-4-yl)propenamide (example 8b)

| | | | 1st eluting | 2nd eluting |
|---|---|---|---|---|
| | Separation | Analysis | diastereomer | diastereomer |
| Example 8 | MD SFC | MD SFC | Example 8a | Example 8b |
| | YMC Amylose-C | YMC Amylose-C | Rt = 2.1 min | Rt = 3.5 min |
| | 40/60 IPA (0.1% | 40/60 IPA (0.1% | | |
| | DEA)/CO₂ | DEA)/CO₂ | | |
| | 100 mL/min | 5 mL/min | | |
| | 40° C. 225 nM; column | 40° C.; column | | |
| | dimensions 250 × 20 | dimensions 250 × 4.6 | | |
| | mm, id 5 μm | mm, id 5 μm | | |

Pharmacological Activity of the Compounds of the invention.

In Vitro Inhibitory Activity Assay Description

The effectiveness of compounds of the present invention to inhibit Rho kinase activity can be determined in a 10 μl assay containing 40 mM Tris pH7.5, 20 mM MgCl₂ 0.1 mg/ml BSA, 50 μM DTT and 2.5 μM peptide substrate (Myelin Basic Protein) using an ADP-Glo kit (Promega). Compounds were dissolved in DMSO such that the final concentration of DMSO was 1% in the assay. All reactions/incubations are performed at 25° C. Compound (2 μl) and either Rho kinase 1 or 2 (4 μl) were mixed and incubated for 30 mins. Reactions were initiated by addition of ATP (4 μl) such that the final concentration of ATP in the assay was 10 μM. After a 1 hour incubation 10 μl of ADP-Glo Reagent was added and after a further 45 minute incubation 20 μl of Kinase Detection Buffer was added and the mixture incubated for a further 30 minutes. The luminescent signal was measured on a luminometer. Controls consisted of assay wells that did not contain compound with background determined using assay wells with no enzyme added. Compounds were tested in dose-response format and the inhibition of kinase activity was calculated at each concentration of compound. To determine the IC₅₀ (concentration of compound required to inhibit 50% of the enzyme activity) data were fit to a plot of % inhibition vs Log₁₀ compound concentration using a sigmoidal fit with a variable slope and fixing the maximum to 100% and the minimum to 0%. To determine the Ki values the Cheng-Prusoff equation was utilized (Ki=IC₅₀/(1+[S]/Km).

Compounds according to the invention showed Ki values lower than 5 μM and for most of the compounds of the invention Ki is even lower that 500 nM.

The results for individual compounds of the examples are provided below in Table 1 and are expressed as range of activity.

TABLE 1

| | Activity | Activity |
|---|---|---|
| Example | ROCK 1 | ROCK 2 |
| 1 | +++ | +++ |
| 2 | +++ | +++ |
| 3 | +++ | +++ |
| 4 | +++ | +++ |
| 5 | +++ | +++ |
| 6 | +++ | +++ |
| 7 | +++ | +++ |
| 8a | +++ | +++ |
| 8b | +++ | +++ |
| 9 | +++ | +++ |
| 10 | +++ | +++ |
| 11 | ++ | +++ |
| 12 | +++ | +++ |
| 13 | +++ | +++ |
| 14 | +++ | +++ |
| 15 | +++ | +++ |
| 16 | +++ | +++ |
| 17 | ++ | ++ |
| 18 | ++ | ++ |
| 19 | +++ | +++ |
| 20 | +++ | +++ |
| 21 | +++ | +++ |
| 22 | +++ | +++ |
| 23 | +++ | +++ |
| 24 | +++ | +++ |
| 25 | +++ | +++ |
| 26 | +++ | +++ |
| 27 | +++ | +++ |
| 28 | +++ | +++ |
| 29 | +++ | +++ |
| 30 | +++ | +++ |
| 31 | +++ | +++ |
| 32 | ++ | +++ |
| 33 | +++ | +++ |
| 34 | +++ | +++ |
| 35 | +++ | +++ |
| 36 | ++ | +++ |
| 37 | +++ | +++ |
| 38 | +++ | +++ |
| 39 | +++ | +++ |
| 40 | +++ | +++ |
| 41 | +++ | +++ |
| 42 | +++ | +++ |
| 43 | +++ | +++ |
| 44 | +++ | +++ |
| 45 | ++ | +++ |
| 46 | +++ | +++ |
| 47 | +++ | +++ |

wherein the compounds are classified in term of potency with respect to their inhibitory activity on ROCK-I and ROCK-II isoforms according to the following classification criterion:

+++: Ki<3 nM

++: Ki in the range 3-30 nM

+: Ki>30 nM

1: A compound of formula (I) 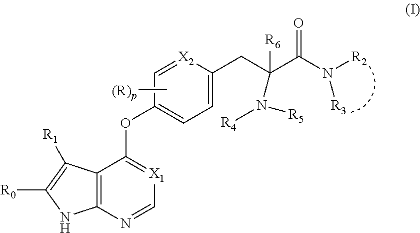
wherein
X₁, and X₂ are in each occurrence independently a CH group or a nitrogen atom,
p is zero or an integer from 1 to 3,
each R, when present, is a halogen;
R₀ and R₁ are independently selected from the group consisting of —H, (C₁-C₆) alkyl, (C₃-C₁₀) cycloalkyl, aryl, heteroaryl and (C₃-C₆) heterocycloalkyl,
each of which aryl, heteroaryl and (C₃-C₆) heterocycloalkyl being in its turn optionally and independently substituted with one or more groups selected from halogen and —OH,
R₂ and R₃, the same or different, are selected from the group consisting of —H, (C₁-C₆) alkyl, (C₁-C₆) haloalkyl, (C₁-C₆) hydroxyalkyl, (C₁-C₆) aminoalkyl, (C₁-C₆) alkoxy (C₁-C₆) alkyl, (C₃-C₁₀)cycloalkyl, (C₃-C₈)heterocycloalkyl, aryl, heteroaryl, aryl(C₁-C₆)alkyl, heteroaryl(C₁-C₆)alkyl, (C₃-C₈)cycloalkyl(C₁-C₆)alkyl, (C₃-C₈)heterocycloalkyl-(C₁-C₆)alkyl,
each of said aryl, heteroaryl, cycloalkyl, heterocycloalkyl is further optionally substituted by one or more group selected independently from halogen, —CN, —OH, (C₁-C₈)alkyl, (C₃-C₆) cycloalkyl, (C₁-C₆) haloalkyl, (C₁-C₁₀)alkoxy, heterocycloalkyl, aryl, aryl(C₁-C₆)alkyl, —C(O)NR₇R₈, (C₁-C₆) aminoalkyl, (C₁-C₆) hydroxyalkyl, (C₁-C₆) alkoxy (C₁-C₆) alkyl, and (C₃-C₈)cycloalkyl(C₁-C₆)alkyl; or
R₂ and R₃, in the alternative, taken together with the nitrogen atom they are linked to, form a mono- or bi-cyclic saturated or partially saturated heterocyclic radical at least one further ring carbon atom in the said heterocyclic radical is optionally replaced by at least one further heteroatom independently selected from N, NH, S or O and/or may bear an -oxo (═O) substituent group, said heterocyclic radical is further optionally including spiro disubstitution as well as substitution on two adjacent or vicinal atoms forming an additional 5 to 6 membered cyclic or heterocyclic, saturated, partially saturated or aromatic, ring;
said heterocyclic radical being optionally in its turn further substituted with one or more groups selected from the group consisting of halogen, —OH, —NR₇R₈, —CH₂NR₇R₈, (C₁-C₆) alkyl, (C₁-C₆)alkyl-sulfonyl, (C₁-C₆) haloalkyl, (C₁-C₆) hydroxyalkyl, (C₂-C₆) alkenyl, (C₂-C₆) alkynyl, (C₂-C₆) hydroxyalkynyl, (C₁-C₆) alkoxy (C₁-C₆) alkyl, (C₁-C₆) alkanoyl, —C(O)NR₇R₈, (C₃-C₆) cycloalkyl-carbonyl, (C₃-C₆) heterocycloalkyl-carbonyl, aryl(C₁-C₆)alkyl, aryl alkanoyl, arylsulfonyl, aryl(C₁-C₆)alkyl-sulfonyl, heteroaryl(C₁-C₆)alkyl, heteroaryl-carbonyl, heteroarylsulfonyl, heteroaryloxyl, (C₃-C₆) cycloalkyl including cycloalkyl-yl, (C₃-C₈)cycloalkyl(C₁-C₆)alkyl, (C₃-C₆) heterocycloalkyl-(C₁-C₆) alkyl, aryl and heteroaryl;
each of said cycloalkyl, aryl and heteroaryl being further optionally substituted by halogen, —OH, (C₁-C₈)alkyl, (C₁-C₆) haloalkyl, (C₁-C₁₀)alkoxy, (C₁-C₆)alkylthio, (C₁-C₆) aminoalkyl, (C₁-C₆) aminoalkoxyl, —C(O)NR₇R₈, and/or (C₁-C₆)alkyl-sulfonyl;
R₄ and R₅ are in each occurrence independently selected in the group consisting of H and (C₁-C₆) alkyl,
R₆ is selected from the group consisting of —H, (C₁-C₆) alkyl, and (C₁-C₆) haloalkyl;
R₇ and R₈ are in each occurrence independently selected in the group consisting of H, (C₁-C₆) alkyl, (C₁-C₆) haloalkyl, (C₁-C₆) hydroxyalkyl, (C₁-C₆) aminoalkyl, (C₁-C₆) alkoxyl, (C₁-C₆) alkoxy-(C₁-C₆) alkyl, (C₃-C₆) heterocycloalkyl-(C₁-C₆) alkyl, (C₃-C₆) cycloalkyl, aryl, heteroaryl and (C₃-C₆) heterocycloalkyl;
wherein any of said aryl, heteroaryl and (C₃-C₆) heterocycloalkyl in its turn is optionally and independently substituted with one or more groups selected from halogen, —OH, and (C₁-C₆) alkyl;
or a pharmaceutically acceptable salt or solvate of said compound.

2: The compound, or salt or solvate thereof, according to claim 1 wherein each of X₁ and X₂ is a CH group.

3: The compound, or salt or solvate thereof, according to claim 1, represented by the formula Ic: 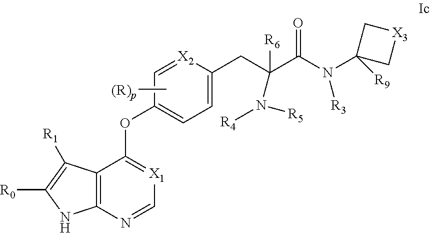
wherein
X₃ is —O— or —(CH₂)ₙ— wherein n is an integer selected from 1, 2 and 3;
R₉ is selected from the group consisting of —C(O)NR₇R₈ and (C₁-C₆) hydroxyalkyl; and
X₁, X₂, R, R₀, R₁, R₃, R₄, R₅, R₆ and p are as defined in claim 1.

4: The compound, or salt or solvate thereof, according to claim 3 wherein
X₁ is CH or N, and X₂ is a CH group;
p is zero or an integer from 1 to 3;
each R, when present, is a halogen;
R₀ is —H;
R₁ is (C₁-C₆) alkyl;
R₃ is —H;
R₄ and R₅ are both H;
R₆ is —H; and
R₉ is-C(O)NR₇R₈, wherein R₇ is H and R₈ is selected from H, (C₁-C₆) alkyl, (C₁-C₆) hydroxyalkyl and (C₁-C₆) alkoxy (C₁-C₆) alkyl.

5: The compound, or salt or solvate thereof, according to claim 1 wherein
X₁ and X₂ are in each occurrence independently a CH group or a nitrogen atom;
p is zero or an integer from 1 to 3;
each R, when present, is fluoro;
R₀ is —H, and R₁ is methyl,
R₃ is —H or methyl and R₂ is independently selected from the group consisting of —H;
methyl;
(C₃-C₁₀)cycloalkyl which is cyclohexyl, cyclobutyl or cyclopentanyl; and
(C₃-C₈)heterocycloalkyl which is piperidinyl, pyranyl or pyrrolidinyl;
each of said cycloalkyl and heterocycloalkyl is further optionally substituted by one or more group selected independently from (C₁-C₈)alkyl which is methyl, ethyl, isobutyl, tert-butyl, or 1-isopropyl; (C₃-C₆) cycloalkyl which is cyclopropyl or cyclobutyl; (C₁-C₆) haloalkyl which is fluoropropyl; heterocycloalkyl which is oxetanyl or tetrahydrofuranyl; —C(O)NR₇R₈ which is aminocarbonyl, methylaminocarbonyl, methoxyethylaminocarbonyl, or hydroxyethylaminocarbonyl; (C₁-C₆) hydroxyalkyl which is hydroxyethyl or hydroxymethyl; (C₁-C₆) alkoxy (C₁-C₆) alkyl which is methoxyethyl; and (C₃-C₈)cycloalkyl(C₁-C₆)alkyl which is cyclopropylmethyl; or
R₂ and R₃, in the alternative, taken together with the nitrogen atom they are linked to, form a mono-cyclic group which is piperidin-N-yl, pyrrolidin-N-yl, or piperazin-N-yl,
or a bi-cyclic group which is 4,7-diazaspiro[2.5]octan-4-yl, (3aR,6aS)-5-cyclopropylhexahydropyrrolo[3,4-c]pyrrol-2(1H)-yl), (1S,4S)-5-cyclopropyl-2,5-diazabicyclo[2.2.1]heptan-2-yl, 3,4-dihydro-2,7-naphthyridin-2(1H)-yl, 5,8-dihydropyrido[3,4-d]pyrimidin-7(6H)-yl, 6,7-dihydrothiazolo[5,4-c]pyridin-5(4H)-yl, or 7,8-dihydro-1,6-naphthyridin-6(5H)-yl; and
R₄, R₅ and R₆ are H.

6: The compound, or salt or solvate thereof, according to claim 1, wherein the compound is selected from the group consisting of:
(S)-2-amino-N-(1-cyclobutylpiperidin-4-yl)-3-(3-fluoro-4-((3-methyl-1H-pyrrolo[2,3-b]pyridin-4-yl)oxy)phenyl)propanamide;
(2S)-2-amino-3-(3-fluoro-4-((3-methyl-1H-pyrrolo[2,3-b]pyridin-4-yl)oxy)phenyl)-N-(1-(tetrahydrofuran-3-yl)piperidin-4-yl)propanamide;
(S)-2-amino-3-(3-fluoro-4-((3-methyl-1H-pyrrolo[2,3-b]pyridin-4-yl)oxy)phenyl)-N-(1-(oxetan-3-yl)piperidin-4-yl)propanamide;
(S)-2-amino-N-(1,4-dimethylpiperidin-4-yl)-3-(3-fluoro-4-((3-methyl-1H-pyrrolo[2,3-b]pyridin-4-yl)oxy)phenyl)propanamide;
(S)-2-amino-N-(1-cyclopropylpiperidin-4-yl)-3-(3-fluoro-4-((3-methyl-1H-pyrrolo[2,3-b]pyridin-4-yl)oxy)phenyl)propanamide;
(S)-2-amino-3-(3-fluoro-4-((3-methyl-1H-pyrrolo[2,3-b]pyridin-4-yl)oxy)phenyl)-N-(1-isobutylpiperidin-4-yl)propanamide;
(S)-2-amino-N-(1-ethylpiperidin-4-yl)-3-(3-fluoro-4-((3-methyl-1H-pyrrolo[2,3-b]pyridin-4-yl)oxy)phenyl)propanamide;
(2S)-2-amino-3-(3-fluoro-4-((3-methyl-1H-pyrrolo[2,3-b]pyridin-4-yl)oxy)phenyl)-N-(1,3,3-trimethylpiperidin-4-yl)propanamide;
(S)-2-amino-3-(3-fluoro-4-((3-methyl-1H-pyrrolo[2,3-b]pyridin-4-yl)oxy)phenyl)-1-(4-hydroxy-4-(hydroxymethyl)piperidin-1-yl)propan-1-one;
(S)-2-amino-3-(3-fluoro-4-((3-methyl-1H-pyrrolo[2,3-b]pyridin-4-yl)oxy)phenyl)-N—((R)-1-methylpyrrolidin-3-yl)propanamide;
(S)-2-amino-3-(3-fluoro-4-((3-methyl-1H-pyrrolo[2,3-b]pyridin-4-yl)oxy)phenyl)-1-(7-methyl-4,7-diazaspiro[2.5]octan-4-yl)propan-1-one;
(S)-2-amino-1-((3aR,6aS)-5-cyclopropylhexahydropyrrolo[3,4-c]pyrrol-2(1H)-yl)-3-(3-fluoro-4-((3-methyl-1H-pyrrolo[2,3-b]pyridin-4-yl)oxy)phenyl)propan-1-one;
(S)-2-amino-1-((1S,4S)-5-cyclopropyl-2,5-diazabicyclo[2.2.1]heptan-2-yl)-3-(3-fluoro-4-((3-methyl-1H-pyrrolo[2,3-b]pyridin-4-yl)oxy)phenyl)propan-1-one;
(S)-2-amino-3-(3-fluoro-4-((3-methyl-1H-pyrrolo[2,3-b]pyridin-4-yl)oxy)phenyl)-N-(1-(2-methoxyethyl)piperidin-4-yl)propanamide;
(S)-2-amino-3-(3-fluoro-4-((3-methyl-1H-pyrrolo[2,3-b]pyridin-4-yl)oxy)phenyl)-N-(1-(3-fluoropropyl)piperidin-4-yl)propanamide;
(S)-2-amino-N-(1-(cyclopropylmethyl)piperidin-4-yl)-3-(3-fluoro-4-((3-methyl-1H-pyrrolo[2,3-b]pyridin-4-yl)oxy)phenyl)propanamide;
First eluted diastereoisomer of (2S)-2-amino-3-(3-fluoro-4-((3-methyl-1H-pyrrolo[2,3-b]pyridin-4-yl)oxy)phenyl)-N-(1-methylpiperidin-3-yl)propanamide;
Second eluted diastereoisomer of (2S)-2-amino-3-(3-fluoro-4-((3-methyl-1H-pyrrolo[2,3-b]pyridin-4-yl)oxy)phenyl)-N-(1-methylpiperidin-3-yl)propanamide;
(S)-2-amino-1-(4-(cyclopropylamino)piperidin-1-yl)-3-(3-fluoro-4-((3-methyl-1H-pyrrolo[2,3-b]pyridin-4-yl)oxy)phenyl)propan-1-one;
(S)-2-amino-N-(1-cyclopropyl-4-(hydroxymethyl)piperidin-4-yl)-3-(3-fluoro-4-((3-methyl-1H-pyrrolo[2,3-b]pyridin-4-yl)oxy)phenyl)propanamide;
(S)-4-(2-amino-3-(3-fluoro-4-((3-methyl-1H-pyrrolo[2,3-b]pyridin-4-yl)oxy)phenyl)propanamido)-1-cyclopropylpiperidine-4-carboxamide;
(S)-2-amino-1-(3,4-dihydro-2,7-naphthyridin-2(1H)-yl)-3-(3-fluoro-4-((3-methyl-1H-pyrrolo[2,3-b]pyridin-4-yl)oxy)phenyl)propan-1-one;
(S)-2-amino-1-(5,8-dihydropyrido[3,4-d]pyrimidin-7(6H)-yl)-3-(3-fluoro-4-((3-methyl-1H-pyrrolo[2,3-b]pyridin-4-yl)oxy)phenyl)propan-1-one;
(S)-2-amino-1-(6,7-dihydrothiazolo[5,4-c]pyridin-5(4H)-yl)-3-(3-fluoro-4-((3-methyl-1H-pyrrolo[2,3-b]pyridin-4-yl)oxy)phenyl)propan-1-one;
(S)-2-amino-1-(3,4-dihydro-2,6-naphthyridin-2(1H)-yl)-3-(3-fluoro-4-((3-methyl-1H-pyrrolo[2,3-b]pyridin-4-yl)oxy)phenyl)propan-1-one;
(S)-2-amino-1-(7,8-dihydro-1,6-naphthyridin-6(5H)-yl)-3-(3-fluoro-4-((3-methyl-1H-pyrrolo[2,3-b]pyridin-4-yl)oxy)phenyl)propan-1-one;
(S)-1-(2-amino-3-(3-fluoro-4-((3-methyl-1H-pyrrolo[2,3-b]pyridin-4-yl)oxy)phenyl)propanamido)cyclobutane-1-carboxamide;
(S)-1-(2-amino-3-(3-fluoro-4-((3-methyl-1H-pyrrolo[2,3-b]pyridin-4-yl)oxy)phenyl)propanamido)-N-methylcyclopentane-1-carboxamide;
(S)-1-(2-amino-3-(3-fluoro-4-((3-methyl-1H-pyrrolo[2,3-b]pyridin-4-yl)oxy)phenyl)propanamido)-N-methylcyclohexane-1-carboxamide;
(S)-2-amino-3-(3-fluoro-4-((3-methyl-1H-pyrrolo[2,3-b]pyridin-4-yl)oxy)phenyl)-N-(1-(hydroxymethyl)cyclobutyl)propanamide;
(S)-2-amino-3-(3-fluoro-4-((3-methyl-1H-pyrrolo[2,3-b]pyridin-4-yl)oxy)phenyl)-1-(4-(m-tolylsulfonyl)piperidin-1-yl)propan-1-one;
(S)-1-(2-amino-3-(3-fluoro-4-((5-methyl-7H-pyrrolo[2,3-d]pyrimidin-4-yl)oxy)phenyl)propanamido)cyclohexanecarboxamide;
(S)-1-(2-amino-3-(3-fluoro-4-((5-methyl-7H-pyrrolo[2,3-d]pyrimidin-4-yl)oxy)phenyl)propanamido)-N-methylcyclohexanecarboxamide;
(S)-2-amino-3-(3-fluoro-4-((5-methyl-7H-pyrrolo[2,3-d]pyrimidin-4-yl)oxy)phenyl)-N-(1-(2-hydroxyethyl)cyclohexyl)propanamide;
(S)-2-amino-3-(3-fluoro-4-((5-methyl-7H-pyrrolo[2,3-d]pyrimidin-4-yl)oxy)phenyl)-N-(1-(hydroxymethyl)cyclohexyl)propanamide;
(S)-2-amino-3-(3-fluoro-4-((5-methyl-7H-pyrrolo[2,3-d]pyrimidin-4-yl)oxy)phenyl)-N-(4-(hydroxymethyl)tetrahydro-2H-pyran-4-yl)propanamide;
(S)-2-amino-3-(3-fluoro-4-((5-methyl-7H-pyrrolo[2,3-d]pyrimidin-4-yl)oxy)phenyl)-1-(4-((4-fluorophenyl)sulfonyl)piperidin-1-yl)propan-1-one;
(S)-2-amino-3-(3-fluoro-4-((5-methyl-7H-pyrrolo[2,3-d]pyrimidin-4-yl)oxy)phenyl)-1-((S)-3-(phenylsulfonyl)pyrrolidin-1-yl)propan-1-one;
(S)-2-amino-N-(1-cyclopropylpiperidin-4-yl)-3-(3-fluoro-4-((5-methyl-7H-pyrrolo[2,3-d]pyrimidin-4-yl)oxy)phenyl)propanamide;
(S)-2-amino-3-(4-((3-cyclopropyl-1H-pyrrolo[2,3-b]pyridin-4-yl)oxy)-3-fluorophenyl)-N-(1-methylpiperidin-4-yl)propanamide;
(S)-2-amino-3-(3,5-difluoro-4-((5-methyl-7H-pyrrolo[2,3-d]pyrimidin-4-yl)oxy)phenyl)-1-(4-(phenylsulfonyl)piperidin-1-yl)propan-1-one;
(S)-2-amino-3-(3,5-difluoro-4-((3-methyl-1H-pyrrolo[2,3-b]pyridin-4-yl)oxy)phenyl)-1-(4-(phenylsulfonyl)piperidin-1-yl)propan-1-one;
(S)-1-(2-amino-3-(3-fluoro-4-((3-methyl-1H-pyrrolo[2,3-b]pyridin-4-yl)oxy)phenyl)propanamido)-N-(2-methoxyethyl)cyclohexanecarboxamide;
(S)-1-(2-amino-3-(3-fluoro-4-((3-methyl-1H-pyrrolo[2,3-b]pyridin-4-yl)oxy)phenyl)propanamido)-N-(2-hydroxyethyl)cyclohexanecarboxamide;
(S)-2-amino-3-(3-fluoro-4-((3-methyl-1H-pyrrolo[2,3-b]pyridin-4-yl)oxy)phenyl)-N,N-dimethylpropanamide;
(S)-2-amino-3-(3-fluoro-4-((3-methyl-1H-pyrrolo[2,3-b]pyridin-4-yl)oxy)phenyl)-N-methylpropanamide;
(S)-2-amino-3-(3-fluoro-4-((3-methyl-1H-pyrrolo[2,3-b]pyridin-4-yl)oxy)phenyl)propanamide;
First eluted diastereoisomer of (2S)-2-amino-3-(3-fluoro-4-((3-methyl-1H-pyrrolo[2,3-b]pyridin-4-yl)oxy)phenyl)-N-(1,3,3-trimethylpiperidin-4-yl)propanamide; and
Second eluted diastereoisomer of (2S)-2-amino-3-(3-fluoro-4-((3-methyl-1H-pyrrolo[2,3-b]pyridin-4-yl)oxy)phenyl)-N-(1,3,3-trimethylpiperidin-4-yl)propenamide.

7: A pharmaceutical composition comprising the compound, or salt or solvate thereof, according to claim 1, in admixture with one or more pharmaceutically acceptable carriers or excipients.

8: The pharmaceutical composition according to claim 7, in a form suitable to be administered by inhalation.

9: A device comprising the pharmaceutical composition according to claim 8, which may be a single- or multi-dose dry powder inhaler, a metered dose inhaler or a soft mist nebulizer.

10. (canceled)

11: A method of treating at least one pulmonary disease selected from the group consisting of asthma, chronic obstructive pulmonary disease (COPD), idiopathic pulmonary fibrosis (IPF), and pulmonary hypertension (PH), the method comprising administering the compound, or salt or solvate thereof, according to claim 1 to a subject in need thereof.

12: A combination of the compound, or salt or solvate thereof, according to claim 1, with one or more active ingredients selected from the classes consisting of organic nitrates and NO donors; inhaled NO; stimulator of soluble guanylate cyclase (sGC); prostaciclin analogue PGI2 and agonist of prostacyclin receptors; compounds that inhibit the degradation of cyclic guanosine monophosphate (cGMP) and/or cyclic adenosine monophosphate (cAMP); human neutrophilic elastase inhibitors; compounds inhibiting the signal transduction cascade; active substances for lowering blood pressure; neutral endopeptidase inhibitor; osmotic agents; ENaC blockers; anti-inflammatories including corticosteroids and antagonists of chemokine receptors; antihistamine drugs; anti-tussive drugs; antibiotics and DNase drug substance and selective cleavage agents; agents that inhibit ALK5 and/or ALK4 phosphorylation of Smad2 and Smad3; tryptophan hydroylase 1 (TPH1) inhibitors and multi-kinase inhibitors.

13: A compound of general formula VI 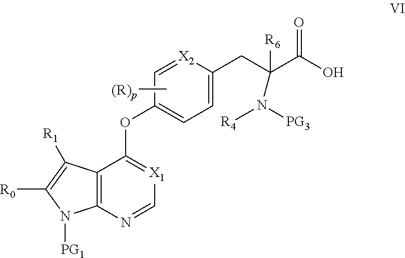
wherein
PG₁ and PG₃ are protecting groups,
X₁, and X₂ are in each occurrence independently a CH group or a nitrogen atom,
p is zero or an integer from 1 to 3,
each R, when present, is a halogen;
R₀ and R₁ are independently selected from the group consisting of —H, (C₁-C₆) alkyl, (C₃-C₁₀) cycloalkyl, aryl, heteroaryl and (C₃-C₆) heterocycloalkyl,
each of which aryl, heteroaryl and (C₃-C₆) heterocycloalkyl being in its turn optionally and independently substituted with one or more groups selected from halogen and —OH,
R₄ is selected from the group consisting of H and (C₁-C₆) alkyl,
R₆ is selected from the group consisting of —H, (C₁-C₆) alkyl, and (C₁-C₆) haloalkyl.

14: The pharmaceutical composition according to claim 8, wherein said form is an inhalable powder, a propellant-containing metering aerosol or a propellant-free inhalable formulation.

15: The method according to claim 11, wherein said pulmonary hypertension (PH) is Pulmonary Arterial Hypertension (PAH).
